SEQUENCE LISTING (92 sequences)

SEQ ID NO: 1 - DNA, 1215 bp, Homo sapiens
  CDS (1)..(1215)
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 2 - protein, 404 aa, Homo sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 3 - DNA, 1197 bp, Homo sapiens
  CDS (1)..(1197)
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 4 - protein, 398 aa, Homo sapiens
MSDSKEPRLQQLGLLEEEQLRGLGFRQTRGYKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 5 - DNA, 939 bp, Homo sapiens
  CDS (1)..(939)
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 6 - protein, 312 aa, Homo sapiens [sequence omitted]

SEQ ID NO: 7 - DNA, 507 bp, Homo sapiens
  CDS (1)..(507)
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 8 - protein, 168 aa, Homo sapiens
MSDSKEPRLQQLGLLEEEQLRGLGFRQTRGYKSLAGCLGHGPLVLQLLSFTLLAGLLVQVSKVPSSISQEQSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 9 - DNA, 1143 bp, Homo sapiens
  CDS (1)..(1143)
atgagtgactccaaggaaccaagactgcagcagctgggcctcctggaggaggaacagctgagaggccttggattccgacagactcgaggatacaagagcttagcagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 10 - protein, 380 aa, Homo sapiens
MSDSKEPRLQQLGLLEEEQLRGLGFRQTRGYKSLAVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 11 - DNA, 1083 bp, Homo sapiens
  CDS (1)..(1083)
atgagtgactccaaggaaccaagactgcagcagctgggcctcctggtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 12 - protein, 360 aa, Homo sapiens
MSDSKEPRLQQLGLLVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 13 - DNA, 807 bp, Homo sapiens
  CDS (1)..(807)
atgagtgactccaaggaaccaagactgcagcagctgggcctcctggtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 14 - protein, 268 aa, Homo sapiens
MSDSKEPRLQQLGLLVSKVPSSISQEQSRQDAIYQNLTQLKAAVGELSEKSKLQEIYQELTQLKAAVGELPEKSKLQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 15 - DNA, 105 bp, Homo sapiens
  CDS (1)..(105)
atgagtgactccaaggaaccaagactgcagcagctgggcctcctggaggaggaacagctgagaggccttggattccgacagactcgtcgtaatcaaaagtgctga

SEQ ID NO: 16 - protein, 34 aa, Homo sapiens
MSDSKEPRLQQLGLLEEEQLRGLGFRQTRRNQKC

SEQ ID NO: 17 - DNA, 1315 bp, Homo sapiens
  CDS (191)..(1315)
atgagtgactccaaggaaccaagactgcagcagctgggcctcctgggtgaggctgggttgggacgctgggattctgggaagggggaagggatggccagccatggcctcagcctgcccaggctctgatttcacgtctatccattcagaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 18 - protein, 374 aa, Homo sapiens
YKSLAGCLGHGPLVLQLLSFTLLAGLLVQVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 19 - DNA, 1243 bp, Homo sapiens
  CDS (101)..(1243)
atgagtgactccaaggaaccaagactgcagcagctgggcctcctgggtgaggctgggttgggacgctgggattctgggaagggggaagggatggccagccatggcctcagcctgcccaggctctgatttcacgtctatccattcagaggaggaacagctgagaggccttggattccgacagactcgaggatacaagagcttagcagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 20 - protein, 380 aa, Homo sapiens
MASACPGSDFTSIHSEEEQLRGLGFRQTRGYKSLAVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 21 - DNA, 1105 bp, Homo sapiens
  CDS (101)..(1105)
atgagtgactccaaggaaccaagactgcagcagctgggcctcctgggtgaggctgggttgggacgctgggattctgggaagggggaagggatggccagccatggcctcagcctgcccaggctctgatttcacgtctatccattcagaggaggaacagctgagaggccttggattccgacagactcgaggatacaagagcttagcagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcgtag

SEQ ID NO: 22 - protein, 334 aa, Homo sapiens
MASACPGSDFTSIHSEEEQLRGLGFRQTRGYKSLAVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTQLKAAVERLCHPCPWEWTFFQGNCYFMSNSQRNWHDSITACKEVGAQLVVIKSAEEQNFLQLQSSRSNRFTWMGLSDLNQEGTWQWVDGSPLLPSFKQYWNRGEPNNVGEEDCAEFSGNGWNDDKCNLAKFWICKKSAASCSRDEEQFLSPAPATPNPPPA

SEQ ID NO: 23 - DNA, 994 bp, Homo sapiens
  CDS (101)..(994)
atgagtgactccaaggaaccaagactgcagcagctgggcctcctgggtgaggctgggttgggacgctgggattctgggaagggggaagggatggccagccatggcctcagcctgcccaggctctgatttcacgtctatccattcagaggaggaacagctgagaggccttggattccgacagactcgaggatacaagagcttagcagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagcttcaagcagtattggaacagaggagagcccaacaacgttggggaggaagactgcgcggaatttag

SEQ ID NO: 24 - protein, 297 aa, Homo sapiens
MASACPGSDFTSIHSEEEQLRGLGFRQTRGYKSLAVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQEIYQELTRLKAAVGELPEKSKQQEIYQELTRLKAAVGELPEKSKQQEIYQELTRLKAAVGELPEKSKQQEIYQELTQLKAAVERLCHPCPWEWTFFQGNCYFMSNSQRNWHDSITACKEVGAQLVVIKSAEEQLQAVLEQRRAQQRWGGRLRGI

SEQ ID NO: 25 - DNA, 547 bp, Homo sapiens
  CDS (101)..(547)
atgagtgactccaaggaaccaagactgcagcagctgggcctcctgggtgaggctgggttgggacgctgggattctgggaagggggaagggatggccagccatggcctcagcctgcccaggctctgatttcacgtctatccattcagaggaggaacagctgagaggccttggattccgacagactcgaggatacaagagcttagcagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcagaacttcacccccttttaa

SEQ ID NO: 26 - protein, 148 aa, Homo sapiens
MASACPGSDFTSIHSEEEQLRGLGFRQTRGYKSLAVSKVPSSISQEQSRQDAIYQNLTQLKAAVGELSEKSKLQEIYQELTQLKAAVGELPEKSKLQEIYQELTRLKAAVGELPEKSKLQEIYQELTWLKAAVGELPEKSKMQNFTPF

SEQ ID NO: 27 - DNA, 1200 bp, Homo sapiens
  CDS (1)..(1200)
atgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 28 - protein, 399 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 29 - DNA, 1648 bp, Homo sapiens
  CDS (1)..(819)
atgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttggtgagttcctgcacatcaagggtccttgggcctgagatggtctctgtgtgatgtgactttacttgagttaccaaccctgcctgagcctcagtttcctcccctgtgaaatgagaacacggggtaactgcgatcattgcacttggtgttcacagtgggccaggcacacagtagacactcagttcaaccaatccgctgccttctgttctgagctctaaagctgctaattctcccagtctggaagagatggagccaggactcctgggttctcctggggatcagggagagtcttgggaagtagggagaggaatggtctctccccattccaagcggtccacccaacatggacagtcaaggaagggccctgattttcaagggcttgaaggctgggcagatatgggaatatggacaacctaatctgagcccagccctgaccagcctcccccaacagaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 30 - protein, 272 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFGEFLHIKGPWA

SEQ ID NO: 31 - DNA, 1535 bp, Homo sapiens
  CDS (1)..(819)
atgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttggtgagttcctgcacatcaagggtccttgggcctgagatggtctctgtgtgatgtgactttacttgagttaccaaccctgcctgagcctcagtttcctcccctgtgaaatgagaacacggggtaactgcgatcattgcacttggtgttcacagtgggccaggcacacagtagacactcagttcaaccaatccgctgccttctgttctgagctctaaagctgctaattctcccagtctggaagagatggagccaggactcctgggttctcctggggatcagggagagtcttgggaagtagggagaggaatggtctctccccattccaagcggtccacccaacatggacagtcaaggaagggccctgattttcaagggcttgaaggctgggcagatatgggaatatggacaacctaatctgagcccagccctgaccagcctcccccaacagaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 32 - protein, 272 aa, Homo sapiens; identical to SEQ ID NO: 30

SEQ ID NO: 33 - DNA, 811 bp, Homo sapiens
  CDS (1)..(699)
atgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccagctgaaggctgcagtggaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 34 - protein, 232 aa, Homo sapiens [sequence omitted]

SEQ ID NO: 35 - DNA, 999 bp, Homo sapiens
  CDS (1)..(999)
atgagtgactccaaggaaccaagggtgcagcagctgggcctcctggaagaagatccaacaaccagtggcatcagactttttccaagagactttcaattccagcagatacatggccacaagagctctacagttccttttcttcttggcccagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtggaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 36 - protein, 332 aa, Homo sapiens
MSDSKEPRVQQLGLLEEDPTTSGIRLFPRDFQFQQIHGHKSSTVPFLLGPVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTELKAAVGELPEKSKLQEIYQELTQLKAAVERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 37 - DNA, 1472 bp, Homo sapiens
  CDS (1)..(756)
atgagtgactccaaggaaccaagggtgcagcagctgggcctcctggaagaagatccaacaaccagtggcatcagactttttccaagagactttcaattccagcagatacatggccacaagagctctacagttccttttcttcttggcccagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttggtgagttcctgcacatcaagggtccttgggcctgagatggtctctgtgtgatgtgactttacttgagttaccaaccctgcctgagcctcagtttcctcccctgtgaaatgagaacacggggtaactgcgatcattgcacttggtgttcacagtgggccaggcacacagtagacactcagttcaaccaatccgctgccttctgttctgagctctaaagctgctaattctcccagtctggaagagatggagccaggactcctgggttctcctggggatcagggagagtcttgggaagtagggagaggaatggtctctccccattccaagcggtccacccaacatggacagtcaaggaagggccctgattttcaagggcttgaaggctgggcagatatgggaatatggacaacctaatctgagcccagccctgaccagcctcccccaacagaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 38 - protein, 251 aa, Homo sapiens
MSDSKEPRVQQLGLLEEDPTTSGIRLFPRDFQFQQIHGHKSSTVPFLLGPVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFGEFLHIKGPWA

SEQ ID NO: 39 - DNA, 792 bp, Homo sapiens
  CDS (1)..(792)
atgagtgactccaaggaaccaagggtgcagcagctgggcctcctggaagaagatccaacaaccagtggcatcagactttttccaagagactttcaattccagcagatacatggccacaagagctctacagttccttttcttcttggcccagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccagctgaaggctgcagtggaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 40 - protein, 263 aa, Homo sapiens
MSDSKEPRVQQLGLLEEDPTTSGIRLFPRDFQFQQIHGHKSSTVPFLLGPVSKVPSSLSQEQSEQDAIYQNLTQLKAAVGELSEKSKLQEIYQELTQLKAAVGELPEKSKLQEIYQELTQLKAAVERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 41 - DNA, 18 bp, Artificial Sequence
  Description of Artificial Sequence  Synthetic Primer
atgagtgactccaaggaa

SEQ ID NO: 42 - DNA, 21 bp, Artificial Sequence
  Description of Artificial Sequence  Synthetic Primer
aagctacagttccttctctcc

SEQ ID NO: 43 - DNA, 21 bp, Artificial Sequence
  Description of Artificial Sequence  Synthetic Primer
gtgaggctgggttgggacgct

SEQ ID NO: 44 - DNA, 21 bp, Artificial Sequence
  Description of Artificial Sequence  Synthetic Primer
ggcctcagcctgcccaggctc

SEQ ID NO: 45 - DNA, 21 bp, Artificial Sequence
  Description of Artificial Sequence  Synthetic Primer
aggaacagctgagaggccttg

SEQ ID NO: 46 - DNA, 24 bp, Artificial Sequence
  Description of Artificial Sequence  Synthetic Primer
tcttggctgggctccttgtccaag

SEQ ID NO: 47 - DNA, 22 bp, Artificial Sequence
  Description of Artificial Sequence  Synthetic Primer
gagcttagcagtgtccaaggtc

SEQ ID NO: 48 - DNA, 29 bp, Artificial Sequence
  Description of Artificial Sequence  Synthetic Primer
cttcaagcagtattggaacagaggagagc

SEQ ID NO: 49 - DNA, 33 bp, Artificial Sequence
  Description of Artificial Sequence  Synthetic Primer
ggtaccaacatctggggacagcgggaaaacatg

SEQ ID NO: 50 - DNA, 25 bp, Artificial Sequence
  Description of Artificial Sequence  Synthetic Primer
gctctagactattcgtctctgaagc

SEQ ID NO: 51 - DNA, 18 bp, Artificial Sequence
  Description of Artificial Sequence  Synthetic Primer
atctggggacagcgggaa

SEQ ID NO: 52 - DNA, 18 bp, Artificial Sequence
  Description of Artificial Sequence  Synthetic Primer
tggagtgaagaagcgctg

SEQ ID NO: 53 - DNA, 18 bp, Artificial Sequence
  Description of Artificial Sequence  Synthetic Primer
ggaggctgaggctagcag

SEQ ID NO: 54 - DNA, 21 bp, Artificial Sequence
  Description of Artificial Sequence  Synthetic Primer
aagatccaacaaccagtggca

SEQ ID NO: 55 - DNA, 1032 bp, Artificial Sequence
  Description of Artificial Sequence  Synthetic Primer
atgagtgactccaaggaaccaagggtgcagcagctgggcctcctggagtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 56 - DNA, 919 bp, Artificial Sequence
  Description of Artificial Sequence  Synthetic Primer
atgagtgactccaaggaaccaagggtgcagcagctgggcctcctggtgtccaaggtccccagctccctaagtcaggaacaatccgagcaagacgcaatctaccagaacctgacccagcttaaagctgcagtgggtgagctctcagagaaatccaagctgcaggggatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 57 - DNA, 1003 bp, Artificial Sequence
  Description of Artificial Sequence  Synthetic Primer
atgagtgactccaaggaaccaagggtgcagcagctgggcctcctggaagaagatccaacaaccagtggcatcagactttttccaagagactttcaattccagcagatacatggccacaagagctctacagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggggagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 58 - DNA, 1047 bp, Artificial Sequence
  Description of Artificial Sequence  Synthetic Primer
atgagtgactccaaggaaccaagggtgcagcagctgggcctcctggaagaagatccaacaaccagtggcatcagactttttccaagagactttcaattccagcagatacatggccacaagagctctacagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaacgaagactgtgcggaatttagtggcagtggctggaacgacaatcgatgtgacgttgacaattactggatctgcaaaaagcccgcagcctgcttcagagacgaatag

SEQ ID NO: 59 - DNA, 1200 bp, Artificial Sequence
  Description of Artificial Sequence  Synthetic Primer
atgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagacygtgcggaatttagtggcagtggctggaacgacaatcgatgtgacgttgacaattactggatctgcaaaaagcccgcagcctgcttcagagacgaatag

SEQ ID NO: 60 - DNA, 1131 bp, Homo sapiens
atgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagctgcagtgggcgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 61 - protein, 376 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 62 - DNA, 1062 bp, Homo sapiens
atgagtgactccaaggaaccaagggtgcagcagctgggcctcctggaagaagatccaacaaccagtggcatcagactttttccaagagactttcaattccagcagatacatggccacaagagctctacagggtgtcttggccatggcgccctggtgctgcaactcctctccttcatgctcttggctggggtcctggtggccatccttgtccaagtgtccaaggtccccagctccctaagtcaggaacaatccgagcaagacgcaatctaccagaacctgacccagcttaaagccgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttacttcatgtctaactcccagcggaactggcacaactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggagcagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 63 - protein, 353 aa, Homo sapiens
MSDSKEPRVQQLGLLEEDPTTSGIRLFPRDFQFQQIHGHKSSTGCLGHGALVLQLLSFMLLAGVLVAILVQVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTQLKAAVGELPDQSKQQQIYQELTDLKTAFERLCRHCPKDWTFFQGNCYFMSNSQRNWHNSVTACQEVRAQLVVIKTAEEQNFLQLQTSRSNRFSWMGLSDLNQEGTWQWVDGSPLSPSFQRYWSSGEPNNSGNEDCAEFSGSGWNDNRCDVDNYWICKKPAACFRDE

SEQ ID NO: 64 - DNA, 1247 bp, Homo sapiens
atgagtgactccaaggaaccaagggtgcagcagctgggcctcctggaagaagatccaacaaccagtggcatcagactttttccaagagactttcaattccagcagatacatggccacaagagctctacagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccagctgaaggctgcagtgggtgagttgccagaccagtccaagcagcagcaaacctatcaagaactgaccgatttgaagactgcatttggtgagttcctgcacatcaagggtccttgggcctgagatggtctctgtgtgatgtgactttacttgagttaccgaccctgcctgagcctcagtttcctcccctgtgaaatgagaacacggggtaactgcgatctttgcacttggtgttcacagtgggccaggcacacagtagacactcagttcaaccaatccgctgccctctgttctgagctctaaagctgctaattctcccagtctggaagagatggagccaggactcctgggttctcctggggatcagggagagtcttgggaagtagggagaggaatggtctctccccattccaagcggtccacgcaacatggacagtcaaggaagggccctgattttcaagggcttgaaggctgggcagatatgggaatatggacaacctaatctgagcccagccctgaccagcctcccccaacagaacgcctgtgccgccactgtcccaaggactggacattcttccaaggaaactgttgcttcatgtctaactcccagcggaactggcacaactccgtcaccgcctgccaggaagtgagggcccagctcgtcgtaatcaaaactgctgaggagcagcttccagcggtactggaacagtggagaacccaacaatagcgggaatgaagactgtgcggaatttagtggcttcagagacgaatag

SEQ ID NO: 65 - protein, 198 aa, Homo sapiens
MSDSKEPRVQQLGLLEEDPTTSGIRLFPRDFQFQQIHGHKSSTVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTQLKAAVGELPDQSKQQQTYQELTDLKTAFGEFLHIKGPWA

SEQ ID NO: 66 - DNA, 1003 bp, Homo sapiens; identical to SEQ ID NO: 57

SEQ ID NO: 67 - protein, 296 aa, Homo sapiens
MSDSKEPRVQQLGLLEEDPTTSGIRLFPRDFQFQQIHGHKSSTVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQLPAVLEQWRTQQ

SEQ ID NO: 68 - DNA, 1047 bp, Homo sapiens; identical to SEQ ID NO: 58

SEQ ID NO: 69 - protein, 348 aa, Homo sapiens
MSDSKEPRVQQLGLLEEDPTTSGIRLFPRDFQFQQIHGHKSSTVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 70 - DNA, 1427 bp, Homo sapiens
atgagtgactccaaggaaccaagggtgcagcagctgggcctcctggaagaagatccaacaaccagtggcatcagactttttccaagagactttcaattccagcagatacatggccacaagagctctacagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttggtgagttcctgcacatcaagggtccttgggcctgagatggtctctgtgtgatgtgcccttacttgagttaccaaccctgcctgagcctcagtttcctcccctgtgaaatgagaaccggggtaactgcgatcattgcacttggtgttcacagtggggccaggcacacagtagacactcagttcaaccaatccgctgccttctgttctgagctctaaagctgctaattctcccagtctggaagagatggagccaggactcctgggttctcctggggatcagggagagtcttgggaagtagggagaggaatggtctctccccattccaagcggtccacccaacatggacagtcaaggaggggccctgattttcaagggcttaaggctgggcagatatgggaatatggacaacctaatctgagcccagccctgaccagcctcccccaacagaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccgcttcagagacgaatag

SEQ ID NO: 71 - protein, 244 aa, Homo sapiens
MSDSKEPRVQQLGLLEEDPTTSGIRLFPRDFQFQQIHGHKSSTVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFGEFLHIKGPWA

SEQ ID NO: 72 - DNA, 978 bp, Homo sapiens
atgagtgactccaggaaaccaagggtgcagcagctgggcctcctggaagaagatccaacaaccagtggcatcagactttttccaagagactttcaattccagcagatacatggccacaagagctctacagtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaaggactggacattcttccaaggaaactgttactttatgtctaactcccagcggaactggcacaactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactgggacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 73 - protein, 325 aa, Homo sapiens
MSDSRKPRVQQLGLLEEDPTTSGIRLFPRDFQFQQIHGHKSSTVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTQLKAAVGELPDQSKQQQIYQELTDLKTAFERLCRHCPKDWTFFQGNCYFMSNSQRNWHNSVTACQEVRAQLVVIKTAEEQNFLQLQTSRSNRFSWMGLSDLNQEGTWQWVDGSPLSPSFQRYWDSGEPNNSGNEDCAEFSGSGWNDNRCDVDNYWICKKPAACFRDE

SEQ ID NO: 74 - DNA, 330 bp, Homo sapiens
atgagtgactccaaggaaccaagggtgcagcagctgggcctcctggaagatccaacaaccagtggcatcagactttttccaaggacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 75 - protein, 109 aa, Homo sapiens
MSDSKEPRVQQLGLLEDPTTSGIRLFPRTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 76 - DNA, 1266 bp, Homo sapiens
atgagtgactccaaggaaccaagggtgcagcagctgggcctcctggtgtccaaggtccccagctccctaagtcaggaacaatccgagcaagacgcaatctaccagaacctgacccagcttaaagctgcagtgggtgagctcccagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggccgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttggcgagttcctgcacatcaagggtccttgggcctgagatggtttctgtgtgatgtgactttacttgagttaccaaccctgcctgagcctcagtttcctcccctgtgaaatgagaacacggggtaactgcgatcattgcacttggtgttcacagtgggccaggcacacagtagacactcagttcaaccaatccgctgccttctgttctgagctctaaagttgctaattctcccagtctggaagagatggagccaggactcctgggttctcctggggatcagggagagtcttgggaagtagggagaggaatggtctctccccattccaagcggtccacccaacatggacagtcaaggaagggccctgattttcaagggcttgaaggctgggcagatatgggaatatggacaacctaatctgagcccagccctgaccagcctcccccaacagaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaagcttcagagacgaatag

SEQ ID NO: 77 - protein, 216 aa, Homo sapiens
MSDSKEPRVQQLGLLVSKVPSSLSQEQSEQDAIYQNLTQLKAAVGELPEKSKLQEIYQELTQLKAAVGELPEKSKLQEIYQELTRLKAAVGELPEKSKLQEIYQELTRPKAAVGELPEKSKLQEIYQELTRLKAAVGELPEKSKLQEIYQELTELKAAVGELPEKSKLQEIYQELTQLKAAVGELPDQSKQQQIYQELTDLKTAFGEFLHIKGPWA

SEQ ID NO: 78 - DNA, 781 bp, Homo sapiens
atgagtgactccaaggaaccaagggtgcagcagctgggcctcctggtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccagctgaaggctgcagtgggtgagttgccagaccagtccaagcagcagcaaatctatcaagaactgaccgatttgaagactgcatttgaacgcctgtgccgccactgtcccaggactggaacattcttccagggaaactgttacttcatgtctaactcccagcggaactggcacgactccgtcaccgcctgccaggaagtgagggcccagctcgtcgtaatcaaaactgctgaggagcagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 79 - protein, 222 aa, Homo sapiens
MSDSKEPRVQQLGLLVSKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQLPAVLEQWRTQQ

SEQ ID NO: 80 - DNA, 919 bp, Homo sapiens; identical to SEQ ID NO: 56

SEQ ID NO: 81 - protein, 268 aa, Homo sapiens
MSDSKEPRVQQLGLLVSKVPSSLSQEQSEQDAIYQNLTQLKAAVGELSEKSKLQGIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQLPAVLEQWRTQQ

SEQ ID NO: 82 - DNA, 1032 bp, Homo sapiens
atgagtgactccaaggaaccaagggtgcagcagctgggcctcctggtgtccaaggtccccagctccctaagtcaggaacaatccgagcaagacgcaatctaccagaacctgacccagcttaaagctgcagtgggtgagctctcagagaaatccaagctgcaggagatctaccaggagctgacccagctgaaggctgcagcgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacccggctgaaggctgcagtgggtgagttgccagagaaatccaagctgcaggagatctaccaggagctgacggagctgaaggctgcagtgggtgagttgccagagaaatccaagctgcaggagatctaccaggagctgacccagctgaaggctgcagtgggtgagttgccagaccagtccaagcagcagcaaatctatcaaggactgaccgatttgaagactgcatttgaacgcctgtgccgccactgtcccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcggacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcgaaaagcccgcagcctgcttcagagacgaatag

SEQ ID NO: 83 - protein, 343 aa, Homo sapiens
MSDSKEPRVQQLGLLESKVPSSLSQEQSEQDAIYQNLTQLKAAVGELSEKSKLQEIYQELTQLKAAVGELPEKSKLQEIYQELTRLKAAVGELPEKSKLQEIYQELTRLKAAVGELPEKSKLQEIYQELTRLKAAVGELPEKSKLQEIYQELTELKAAVGELPEKSKLQEIYQELTQLKAAVGELPDQSKQQQIYQELTDLKTAFERLCRHCPKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 84 - DNA, 405 bp, Homo sapiens
atgagtgactccaaggactggacattcttccaaggaaactgttacttcatgtctaactcccagcggaactggcacgactccgtcaccgcctgccaggaagtgagggcccagctcgtcgtaatcaaaactgctgaggagcagaacttcctacagctgcagacttccaggagtaaccgcttctcctggatgggactttcagacctaaatcaggaaggcacgtggcaatgggtggacggctcacctctgtcacccagcttccagcggtactggaacagtggagaacccaacaatagcgggaatgaagactgtgcggaatttagtggcagtggctggaacgacaatcgatgtgacgttgacaattactggatctgcaaaaagcccgcagcctgcttcagagacgaatag

SEQ ID NO: 85 - protein, 134 aa, Homo sapiens
MSDSKDWTFFQGNCYFMSNSQRNWHDSVTACQEVRAQLVVIKTAEEQNFLQLQTSRSNRFSWMGLSDLNQEGTWQWVDGSPLSPSFQRYWNSGEPNNSGNEDCAEFSGSGWNDNRCDVDNYWICKKPAACFRDE

SEQ ID NO: 86 - DNA, 534 bp, Homo sapiens
atggagcaggagagcgccctgcgggaactgcgtgagcgcgtgacccagggcttggctgaagccggcaggggccgtgaggacgtccgcactgagctgttccgggcgctggaggccgtgaggctccagaacaactcctgcgagccgtgccccacgtcgtggctgtccttcgagggctcctgctactttttctctgtgccaaagacgacgtgggcggcggcgcaggatcactgcgcagatgccagcgcgcacctggtgatcgttgggggcctggatgagcagggcttcctcactcggaacacgcgtggccgtggttactggctgggcctgagggctgtgcgccatctgggcaaggttcagggctaccagtgggtggacggagtctctctcagcttcagccactggaaccagggagagcccaatgacgcttgggggcgcgagaactgtgtcatgatgctgcacacggggctgtggaacgacgcaccgtgtgacagcgagaaggacggctggatctgtgagaaaaggcacaactgctga

SEQ ID NO: 87 - protein, 177 aa, Homo sapiens [sequence omitted]

SEQ ID NO: 88 - DNA, 708 bp, Homo sapiens
atggacaccaccaggtacagcaagtggggcggcagctccgaggaggtccccggagcctccacggagcgcgcggcgctgcttgacggccacgacctgctgaggacaaacggctcggggacgcaggcgcagctgcagaccacgcgcgcggagcttggggaggcgcaggcgaagctgatggagcaggagagcgccctgcgggaactgcgtgagcgcgtgacccagggcttggctgaagccggcaggggccgtgaggacgtccgcactgagctgttccgggcgctggaggccgtgaggctccagaacaactcctgcgagccgtgccccacgtcgtggctgtccttcgagggctcctgctactttttctctgtgccaaagacgacgtgggcggcggcgcaggatcactgcgcagatgccagcgcgcacctggtgatcgttgggggcctggatgagcagggcttcctcactcggaacacgcgtggccgtggttactggctgggcctgagggctgtgcgccatctgggcaaggttcagggctaccagtgggtggacggagtctctctcagcttcagccactggaaccagggagagcccaatgacgcttgggggcgcgagaactgtgtcatgatgctgcacacggggctgtggaacgacgcaccgtgtgacagcgagaaggacggctggatctgtgagaaaaggcacaactgctga

SEQ ID NO: 89 - DNA, 970 bp, Homo sapiens
gctgatggagcaggagagcgccctgcgggaactgcgtgagcgcgtgacccagggcttggctgaagccggcaggggccgtgaggacgtccgcactgagctgttccgggcgctggaggccgtgaggctccagaacaactcctgcgagccgtgccccacgtcgtggctgtccttcgagggctcctgctactttttctctgtgccaaagacgacgtgggcggcggcgcaggatcactgcgcagatgccagcgcgcacctggtgatcgttgggggcctggatgagcagggcttcctcactcggaacacgcgtggccgtggttactggctgggcctgagggctgtgcgccatctgggcaaggttcagggctaccagtgggtggacggagtctctctcagcttcagccactggaaccagggagagcccaatgacgcttgggggcgcgagaactgtgtcatgatgctgcacacggggctgtggaacgacgcaccgtgtgacagcgagaaggacggctggatctgtgagaaaaggcacaactgctgaccccgcccagtgccctggagccgcgcccattgcagcatgtcgtatcctgggggctgctcacctccctggctcctggagctgattgccaaagagttttttcttcctcatccaccgctgctgagtctcagaaacacttggcccaacatagccctgtccagcccagtgcctgggctctgggacctccatgccgacctcatcctaactccactcacgcagacccaacctaacctccactagctccaaaatccctgctcctgcgtccccgtgatatgcctccacttctctccctaaccaaggttaggtgactgaggactggagctgtttggttttctcgcattttccaccaaactggaagctgtttttgcagcctgaggaagcatcaataaatatttgagaaatgaaaaaaaaaaaaaaaaaaaaaaaaaaaaaaaa

SEQ ID NO: 90 - DNA, 217 bp, Homo sapiens
agctgatggagcaggagagcgccctgcgggaactgcgtgagcgcgtgacccagtgcttggctgaagccggcaggggccgtgagtgacgtccgcactgagctgttccgggcgctggaggccgtgaggctccagaacaactcctgcgagccgtgccccacgtcgtggctgtccttcgaggtgctcctgctactttttctctgtgccaaagacgacgtgg

SEQ ID NO: 91 - DNA, 214 bp, Homo sapiens
agctgatggagcaggagagcgccctgcgggaactgcgtgagcgcgtgacccaggcttggctgaagccggcaggggccgtgaggacgtccgcactgagctgttccgggcgctggaggccgtgaggctccagaacaactcctgcgagccgtgccccacgtcgtggctgtccttcgagggctcctgctactttttctctgtgccaaagacgacgtgg

SEQ ID NO: 92 - protein, 21 aa, Artificial Sequence
  Description of Artificial Sequence  Synthetic Peptide
CSRDEEQFLSPAPATPNPPPA

(19) United States
(12) Patent Application Publication
(10) Pub. No.: US 2005/0221291 A1
(43) Pub. Date: Oct. 6, 2005

(54) Dc-sign isoforms, related compositions and methods for their use in disease therapy

(75) Inventors: Sunil K. Ahuja, San Antonio, TX (US); Seema Ahuja, San Antonio, TX (US); Srinivas Mummidi, San Antonio, TX (US); Gabriel Catano, San Antonio, TX (US)

Correspondence Address: FULBRIGHT & JAWORSKI L.L.P., 600 CONGRESS AVE., SUITE 2400, AUSTIN, TX, 78701, US

(73) Assignee: Board of Regents, The University of Texas System, Austin, TX (US)

(21) Appl. No.: 10/513,082

(22) PCT Filed: May 5, 2003
(86) PCT No.: PCT/US03/13916

§ 371 Date: Jun. 7, 2005

Related U.S. Application Data
(60) Provisional application No. 60/377,609, filed on May 3, 2002.

(51) Int. Cl. C12Q 1/70; C07H 21/04; C12N 15/09; C07K 14/705

(52) U.S. Cl. 435/5; 435/69.1; 435/320.1; 435/325; 530/350; 536/23.5

(57) ABSTRACT

The present invention provides polypeptides of DC-SIGN 1, DC-SIGN2 and DC-SIGN3 isoforms, nucleic acids encoding these polypeptides, and methods of use in treating disease and modulating immune responses.

### BACKGROUND OF THE INVENTION

This application claims priority to U.S. Provisional Application Ser. No. 60/377,609, filed May 3, 2000, the entire text of which is specifically incorporated herein by reference without disclaimer.

### A. FIELD OF THE INVENTION

The present invention relates to compositions and methods comprising DC-SIGN1, DC-SIGN2 and DC-SIGN3 isoforms and nucleic acids encoding such isoforms for use in modulating immune responses, genetic transformation, and treatment of disease.

### B. RELATED ART

The dissemination of human immunodeficiency virus (HIV-1)¹ and establishment of infection within an individual involve the transfer of virus from mucosal sites of infection to T cell zones in secondary lymphotic organs. How this happens not precisely known. However, there is growing support for the notion that dendritic cells (DCs) present within the mucosal sites may play a central role in this process (Banchereau and Steinman, 1998 Knight and Patterson, 1997; Weissman and Fauci, 1997; granilli-Piperno et al., 1996; Granelli-Piperno et al., 1999; Zaitseva et al., 1997; Canque et al., 1999, Blauvelt et al., 1997; Cimarelli et al., 1994; Pinchuk et al., 1994; Tsunetsugu-Yokota et al., 1995; Tsunetsugu-Yokota et al., 1997; Frank et al., 1999 Kacani et al., 1998; Steinman, 2000). The normal function of DCs is to survey mucosal surfaces for antigens, capture the antigens, process captured proteins into immunogenic peptides, emigrate from tissues to the paracortex of draining lymph nodes, and present peptides in the context of MHC (major histocompatibility complex) molecules to T cells (Banchereau and Steinman, 1998). It is now generally believed that HIV1 may subvert this normal trafficking process to gain entry into lymph nodes and access to CD4⁺ T cell. There is also evidence demonstrating that productive infection of DCs and the ability of DCs to capture virus with subsequent transmission to T cells is mediated through two separate pathways (Granelli-Piperno et al., 1999; Blauvelt et al. 1997; Weissman and Fauci 1997; Steinman 2000). Thus, strategies designed to block mucosal transmission of HIV will require a clear understanding of the molecular determinants of not only virus infection but also of virus capture by DCs or other cell types that can subserve a similar function.

Two recent reports by Geijtenbeek et al. (Geijtenbeek et al., 2000; Geijtenbeek et al., 2000) demonstrated that a mannose-binding, C-type lectin designated as SIGN (DC specific, ICAM-3 grabbing, nonintegrin) may play a key role in DCT cell interactions as well as in HIV pathogenesis. First, by binding to ICAM-3 expressed on T cells, DC-SIGN is thought to facilitate the initial interaction between DCs and naive T cells (Geijtenbeek et al., 2000), setting the stage for subsequent critical events that lead to antigen recognition and the formation of a contact zone termed the immunological synapse (Steinman, 2000). Second, HIV-1 may exploit DC-SIGN for its transport via DCs from mucosal surfaces to secondary lymphoid organs rich in activated memory CD4+ T cells that express CC chemokine receptor 5 (CCR5). Unlike CCR5, the major co-receptor for HIV-1 cell entry, DC-SIGN is not a co-receptor for viral entry. Geijtenbeek et al (2000) confirmed an earlier observation that DC-SIGN is an HIV-1 envelope (gp120)-binding lectin (Curtis et al., 1992) and extended significantly this finding by showing that it promotes efficient infection in trans of cells that express CD4 and CCR5. This delivery and subsequent transmission of HIV in a DC-SIGN-dependent manner to viral replication-permissive T cells may play a major role in viral replication, especially at low concentrations of HIV (Geijtenbeek et al., 2000).

The interest in DC-SIGN stems from the studies that focus on understanding the host genetic determinants of HIV-1 pathogenesis. For example, the inventors have demonstrated that polyps in the gene for CCR5 influence the rate of disease progression in infected adults and children and in mother-to-child transmission (Gonzalez et al., 1999; Mangano et al., 2001). Further interest stems for the role that DC-SIGN may play in pathogenesis of the Ebola virus. Studies show that viruses with the Ebola virus glycoprotein are susceptible to inhibition of infection by administration of antibodies to DC-SIGN1 or DC-SIGN2 (Pohlmann, et al. 2002).

### SUMMARY OF THE INVENTION

In a particular embodiment the present invention is directed to an isolated and purified isoform of DC-SIGN1, DC-SIGN2 or DC-SIGN3. Among the embodiments of the invention are isoforms comprising an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87 and SEQ ID NO: 92.

In a particular aspect, the invention is embodied by an isoform that is shorter than the full length DC-SIGN1 and DC-SIGN2 isoforms.

The isoforms of DC-SIGN1 and DC-SIGN2 that are embodied in the present invention may comprise various deletions, alterations, ions, and other modifications of the amino acid sequences of the prototypic DC-SIGN1 and DC-SIGN2 molecules. Many of the isoforms are comprised of polypeptides formed of amino acid sequences with particular functions. These polypeptides may be designated as domains. Thus, DC-SIGN1 and DC-SIGN2 isoforms comprise, variously, a cytoplasmic domain, a transmembrane domain, a neck region domain, a lectin binding domain, a carbohydrate recognition domain, and additional polypeptide domains that may result from the extensive diversity of DC-SIGN isoforms generally.

Thus, in a particular embodiment the isoform may comprise a carbohydrate recognition domain (CRD). In an additional embodiment, the isoform comprises a lectin binding domain. In a further embodiment, the isoform comprises from 1 to 8 repeats in the neck domain another embodiment the isoform comprises a transmembrane domain. In an especially preferred embodiment, the DC-SIGN isoform lacks a transmembrane domain and is thereby rendered soluble. In yet a further aspect the isoform comprises a cytoplasimic (CYT) domain.

These domains may be variously combined to achieve the goals of the invention, depending on the particular application desired by the artisan. Thus, complete and partial domains may be created with particular properties and activities, all within the scope and content of the present invention as will be recognized by one of skill in the relevant art

Further, preferred embodiments include isoforms based upon the DC-SIGN1 prototypical isoform and separately, the DC-SIGN2 or DC-SIGN 3 prototypical isoform. Therefore, particular embodiments include DC-SIGN1 isoforms comprising an amino acid sequence selected from the group consisting of SEQ ID NO: 2 , SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, and SEQ ID NO: 26.

Further, particular embodiments include DC-SIGN2 isoforms comprising an amino acid sequence selected from the group consisting of SEQ ID NO: 28, SEQ ID NO: 30, SEQ ID NO: 32, SEQ ID NO: 34, SEQ ID NO: 36, SEQ ID NO: 38, and SEQ ID NO: 40 and all other sequences listed in the sequence listing section of this application.

Additionally, fusion, or chimeric isoforms are especially contemplated embodiments of the invention, wherein the isoforms comprise, variously, fusions of extracellular, CDR, neck repeat, transmembrane, intracellular domain, and ICAM binding domains of a DC-SIGN isoform in which at least one domain is taken from a DC-SIGN isoform other than the DC-SIGN isoforms from which the remaining domains are taken.

In a particular embodiment, the isoform is an isoform of DC-SIGN1 comprising all or part of the contiguous amino acid sequence encoded by the nucleic acid sequence of Exon Ib of DC-SIGN1. In another particular embodiment, the isoform is translated from nucleotide position +101 in Exon 1b of DC-SIGN1. In an additional aspect, the DC-SIGN1 isoform has fewer than 8 neck repeats.

In an especially preferred embodiment the isoform is a soluble isoform of DC-SIGN1. In a further preferred embodiment, the isoform is a soluble isoform of DC-SIGN1 comprising all or part of the contiguous amino acid sequence encoded by the nucleic acid sequence of Exon 1b of DC-SIGN1.

A further embodiment is an isoform of DC-SIGN1 comprising an ICAM binding domain. In one embodiment, the isoform has 8 repeats in the neck domain. Another embodiment is wherein the isoform is an isoform of DC-SIGN1 comprising fewer than 8 complete neck repeats. A particularly preferred embodiment is wherein the isoform comprises the amino acid sequence of SEQ ID NO:6.

In yet a further preferred embodiment, the isoform is an isoform of DC-SIGN2 or DC-SIGN3. In an additional aspect, the isoform is a membrane bound isoform of DC-SIGN. In a further aspect, the isoform is a DC-SIGN2 or DC-SIGN 3 isoform with fewer than 8 neck repeats.

In a particularly preferred embodiment, the isoform is a soluble isoform of DC-SIGN2 or DC-SIGN3. In another embodiment, the isoform is a soluble isoform of DC-SIGN2 comprising all or part of the contiguous amino acid sequence encoded by the nucleic acid sequence of Exon IVa of DC-SIGN2. In one aspect, this isoform may have fewer than 8 repeats in the neck region. In an especially preferred embodiment, the isoform is a soluble isoform of DC-SIGN2 comprising an ICAM binding domain. In an additional aspect, the isoform may have fewer than 8 neck region repeats.

In an additional embodiment, the present invention is directed to an isolated and purified nucleic acid encoding an isoform of DC-SIGN1, DC-SIGN2 or DC-SIGN3. The particular isoforms encoded by the nucleic acid embodiments of the present invention may be determined by the particular domains included in the isoform as described above. Since the nucleic acids of the present invention may encode the various combinations of domains and isoforms described above and throughout the specification, the particular nucleic acid sequence is not thought to be necessarily limiting. Among the embodiments of the invention are nucleic acids wherein the nucleic acid encodes a sequence selected from the grouping of SEQ ID NO: 1, SEQ ID NO: 3, SEQ ID NO: 5, SEQ ID NO: 7,SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39 and all other sequences listed in the sequence listing filed herewith.

In a particular embodiment, the invention includes a nucleic acid that encodes an isoform shorter than the full length DC-SIGN1, DC-SIGN2 and/or DC-SIGN3 isoforms. In additional embodiments, the nucleic acid encodes an isoform comprising a carbohydrate recognition domain (CRD). In another aspect the nucleic acid encodes an isoform comprising a lectin binding domain. In another aspect, the nucleic acid encodes an isoform comprising a neck repeat region comprising from 1 to 8 repeats. In yet a further aspect, the nucleic acid encodes an isoform that comprises a cytoplasmic (CYT) domain. In a particular embodiment, the nucleic acid encodes an isoform comprising a transmembrane domain.

In an especially preferred embodiment the nucleic acid encodes an isoform lacking a transmembrane domain.

In several embodiments, the nucleic acid encodes an isoform of DC-SIGN1. Thus, the nucleic acid may comprise a sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, and SEQ ID NO: 25.

In a particular embodiment, the nucleic acid the nucleic acid encodes an isoform of DC-SIGN1 comprising all or part of the contiguous amino acid sequence encoded by Exon 1b of DC-SIGN1. In a further aspect, the nucleic acid encodes an isoform that is translated from position +101 in Exon 1b of DC-SIGN1. In further preferred embodiments, the nucleic acid encodes an isoform of DC-SIGN1 comprising an ICAM binding domain.

Of course, an additional embodiment includes a nucleic acid encoding an isoform of DC-SIGN2. In particular embodiments, such nucleic acids may comprise a sequence selected from the group comprising of SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82 and SEQ ID NO: 84. In a particularly preferred embodiment, the nucleic acid encodes a membrane bound isoform of DC-SIGN2 and may encode a DC-SIGN2 isoform with less than 8 neck repeats. Other DC-SIGN 2 isoforms are identified in the sequence listing and contemplated by the inventors.

In an especially preferred embodiment, the nucleic acid encodes a soluble isoform of DC-SIGN2. In a further embodiment the nucleic acid encodes a soluble isoform of DC-SIGN2 and comprises Exon IVa of DC-SIGN2. Another embodiment includes a nucleic acid that encodes a soluble isoform of DC-SIGN2 with fewer than 8 neck repeats. In a her aspect a nucleic acid encodes a soluble isoform of DC-SIGN2 comprising an ICAM binding domain. In yet a further aspect, the nucleic acid encodes a soluble isoform of DC-SIGN2 with fewer than 8 neck region repeats.

Additional, the nucleic acid encodes a soluble isoform of DC-SIGN3. In particular embodiments, the nucleic acid sequence may comprise the sequence of SEQ ID. No. 86.

An additional embodiment of the invention is a cell transformed with any one or more of the nucleic acids described herein. In an additional aspect an embodiment is a cell wherein the cell expresses the DC-SIGN isoform encoded by the nucleic acid.

Yet a further and preferred embodiment of the present invention is a method for treating disease comprising administering a therapeutically effective amount of an isoform of DC-SIGN1, DC-SIGN2 or DC-SIGN3 to a subject in need of such treatment

In further aspects, the disease is selected from caner, viral infection, or non-HIV induced immunosuppression. In preferred embodiments the disease is viral infection. In a particularly preferred embodiment the viral infection is HIV infection. In yet another preferred embodiment, the viral infection is Ebola virus infection.

Further embodiments include methods of treatment of disease wherein the isoform is a DC-SIGN1 isoform, a DC-SIGN2 isoform, a DC-SIGN3 isoform or a combination of isoforms. The isoforms may be soluble, and may comprises a carbohydrate recognition domain and/or additionally an ICAM binding or lectin binding domain. Thus, particularly preferred embodiments include soluble isoforms of either or all DC-SIGN1, DC-SIGN2 or DC-SIGN3 employed in the treatment of disease wherein the isoforms comprise useful combinations of DC-SIGN domains.

Further embodiments of the invention include methods of treatment of disease within the DC-SIGN isoform binds to a host protein.

Embodiments of the present invention also include methods of modulating an immune response comprising providing an amount of one or more DC-SIGN isoforms sufficient to enhance or inhibit the immune response. In a further aspect, the method modulates a T-cell mediated immune response. In a preferred embodiment the method employs a DC-SIGN isoform to inhibit or attenuate a T-cell mediated immune response. In a further aspect the DC-SIGN isoform interacts with a T cell surface receptor. The embodiments of this invention include methods employing more than one DC-SIGN isoform at once in the modulation of an immune response or treatment of disease.

In further embodiments, the methods of treatment and methods of immunomodulation are embodied by an antibody that binds a DC-SIGN1, DC-SIGN2 or DC-SIGN3 isoform. In an additional aspect the antibody is a polyclonal antibody. In another aspect the antibody is a monoclonal antibody. In yet a further aspect, the antibody is a humanized antibody.

In still further embodiments, the preset invention thus concerns immunodetection methods for binding, purifying, removing, quantifying or otherwise germ detecting biological components. The encoded proteins or peptides of the present invention may be employed to detect antibodies having reactivity therewith, or, alternatively, antibodies prepared in accordance with the present invention, may be employed to detect the encoded proteins or peptides, such as any of the DC-SIGN isoforms.

In yet a further embodiment the invention is a method of modulating resistance to viral infection comprising identifying a subject at risk for a viral infection and administering to the subject a composition comprising a DC-SIGN isoform, a fusion protein containing a DC-SIGN domain or an antibody to DC-SIGN in an amount sufficient to alter the resistance of the subject to the viral infection.

In yet a further embodiment, the invention is a method of augmenting transformation of ICAM expressing cells comprising a) obtaining a cell that expresses ICAM on its surface; b) obtaining a viral vector, and c) contacting the cell of step b) with the vector of step a) in the presence of a DC-SIGN isoform such that the vector is incorporated into the cell.

In a further aspect the cell that expresses ICAM on its surface is a T cell. In an additional embodiment, the vector comprises at last one glycoprotein that is homologous to the vector. In a particular embodiment the glycoprotein is gp120. In an additional embodiment, the vector comprises a transgene. In a further embodiment, the transgene is a therapeutic gene. In a preferred aspect, the DC-SIGN isoform is expressed on the surface of a cell employed in cellular transformation.

A further embodiment is a cell transformed by such methods.

In yet a further embodiment, the invention is a method of assaying for susceptibility to disease comprising a) obtaining a sample from a subject to be assayed; b) identifying the DC-SIGN type present in the sample; and c) determining the susceptibility of the subject to disease based upon a correlation of DC-SIGN type and susceptibility.

In additional embodiments, the invention is a method of assaying for susceptibility to disease wherein the DC-SIGN type is a DC-SIGN isoform. In a further aspect, the DC-SIGN type is identified by selective binding of an antibody specific for one or more DC-SIGN isoforms. In yet a further preferred embodiment, the DC-SIGN type is identified by ELISA.

In an additional embodiment, the invention is a method of assaying for susceptibility to disease wherein the DC-SIGN type is a DC-SIGN associated haplotype. In a further aspect, the DC-SIGN haplotype is identified by RT-PCR.

In yet a further embodiment, the invention is a method of assaying for susceptibility to disease comprising obtaining a sample from a subject to be assayed; obtaining at least a second sample from the subject to be assayed; identifying the DC-SIGN type present in each of the sample; and determining the susceptibility of the subject to disease based upon a correlation of DC-SIGN type profile across samples and susceptibility. In a further aspect, these samples are derived from different tissues of the same subject.

Another embodiment of the invention is a method of screening for modulators of DC-SIGN activity comprising: a) providing a candidate modulator; b) admixing the candidate modulator with DC-SIGN and additional molecules or cells or animals; c) measuring one or more characteristics of the additional molecules or cell in step (b); and d) comparing the characteristic measured in step (c) with the characteristic of the compound or cell or animal in the absence of said candidate modulator, wherein a difference between the measured characteristics indicates that said candidate modulator is a modulator of the compound, cell or animal.

Another embodiment of the invention is a kit for modulating an immune response comprising a DC-SIGN isoform. A further embodiment includes a kit comprising a soluble DC-SIGN isoform.

Another embodiment of the invention is a kit for treating disease comprising a modulator of DC-SIGN activity. Another embodiment of the invention is a kit for determining the disease susceptibility of a subject mediated by DC-SIGN type.

A further embodiment of the invention is a method of treating disease comprising: a) identifying a subject in need of treatment; b) obtaining a cell; c) transforming the cell with a nucleic acid encoding a DC-SIGN isoform; and d) administering the cell to the subject.

Following long-standing patent law, the words “a” and “an,” when used in conjunction with the word “comprising” in the claims or specification, denotes one or more.

### BRIEF DESCRIPTION OF THE DRAWINGS

The following drawings from part of the present specification and are included to further demonstrate certain aspects of the present invention. The invention may be better understood by reference to one or more of these drawings in combination with the detailed description of specific embodiments presented herein.

FIG. 1A. Molecular basis of the extensive repertoire of DC-SIGN isoforms and schematic illustration of the molecular basis for generation of DC-SIGN1A mRNA transcripts. Topmost panel is a schematic illustration of the DC-SIGN1 gene. Horizontal lines are exons (I-VI) and dashed lines illustrate the splicing events that lead to the formation of the prototypic exon II-containing DC-SIGN1A mRNA transcript that was originally described by Curtis et al., and designated herein as mDC-SIGN1A Type I. +1 indicates the translational start site in this prototypic DC-SIGN1 mRNA. Exon II encodes the transmembrane (TM) domain (FIG. 2), and the exon II-containing DC-SIGN1A mRNA transcripts encode membrane-bound or mDC-SIGN1A isoforms, whereas mRNAs that lack this TM-encoding exon II encode soluble or sDC-SIGN1A isoforms. Alternative splicing events that lead to the generation of mRNA transcripts that contain or lack the TM-encoding exon II can be deduced by joining the various exonic sequences indicated; the starting and ending nucleotide number of each exonic segment is separated by dots (e.g. join 1 . . . 46), and exonic segments are separated from each other by a comma (e.g. 1 . . . 46, 147 . . . 206, 981 . . . 1052). Note that the inventors did not determine the length of the 5′ untranslated region (UTR) of DC-SIGN1. sDC-SIGN1A Type I represents the prototypic exon II-lacking DC-SIGN1A mRNA. Note, the translation initiation codon for all DC-SIGN1A (Type I) or sDC-SIGN1A (Type I) mRNA transcripts. Asterisk indicates the stop codon used by the DC-SIGN1A transcripts shown in this panel. The numbering system is based on the nucleotide sequence deposited under GenBank™ accession number AC008812, and the first nucleotide of the initiation Met codon of the prototypic mDC-SIGN1 (Type I) mRNA is considered as +1.

FIG. 1B. Molecular basis for generation of DC-SIGN1B mRNA transcripts. Topmost panel is a schematic illustration of DC-SIGN1 gene. Horizontal lines are exons (I-VI). Exon Ib is exonic sequences separating exon Ia and Ic sequences, and all DC-SIGN1 mRNAs that contain exon Ib are designated as DC-SIGN1B mRNA transcripts. Sequence analysis of exon Ib containing transcripts revealed two potential translation initiation sites (+1 or +101). Transcripts predicted to initiate translation at +101 in exon Ib may contain or lack the TM-encoding exon II. Dashed lines illustrate the splicing events that lead to formation of the prototypic exon II-containing DC-SIGN1B mRNA transcript (mDC-SIGN1B Type I). Splicing out of the TM-encoding exon II generates transcripts designated as sDC-SIGN1B Types HV (Type I is the prototype). Positions in bold denote a splicing site that is distinct from that found in the prototypic mDC-SIGN1B mRNAs. The stop codons utilized by sDC-SIGN1B types III and IV at position 4335-4337 and 4492-4494 respectively are indicated by daggers, and the positions 4334 and 4491 are underlined. The DC-SIGN1B transcripts that also initiate translation at +1 in exon Ia have an in-frame stop codon (TGA; +124-126); these transcripts generate a short polypeptide of 41 aa (see FIGS. 2 and 3).

FIG. 1C. Non-canonical splice donor and/or acceptor sites used in generation of some DC-SIGN1 mRNA transcripts.

FIG. 2A. Structure and molecular diversity of membrane-bound and soluble DC-SIGN1 gene products with novel intra- and/or extra-cellular domains and Gene organization of DC-SIGN1 and alternative splicing events that lead to the generation of the prototypic DC-SIGN1 protein product described by Curtis et al. (1992). Boxes are exons (I-VI) and dashed lines are introns (I-V in black circles). The nucleotide length of the introns are shown in parentheses. The first nucleotide of the initiation Met codon of the prototypic DC-SIGN1 is considered as +1. The stop codon used by the prototypic DC-SIGN1A isoform is denoted by an asterisk. The box with vertical hatch lines represents a small portion of the predicted 3′-untranslated region (UTR), and some DC-SIGN1B transcripts terminate translation at position 4491 in this region (FIG. 1). The prototypic DC-SIGN1 protein product comprises a short cytoplasmic (CYT; open boxes) and transmembrane (TM; box with forward slash) domain. Exons III-VI encode the extracellular (EC) domain of the prototypic DC-SIGN1 and this includes a short stretch of sequence just proximal to the repeals (box with horizontal lines), the seven full repeats and one half repeat (numbered black boxes), and the lectin-binding domain (backward slash). The shaded box represents the alternatively spliced exon Ib.

FIG. 2B, FIG. 2C, FIG. 2D, AND FIG. 2E. Schematic illustrations of the molecular diversity and predicted structures of DC-SIGN1A and DC-SIGN1 B isoforms generated by alternative splicing events. DC-SIGN1 variants that lack or contain exon Ib sequences are designated as DC-SIGN1A FIG. 2D AND FIG. 2E isoforms, respectively (FIG. 1). Amino acid differences among the isoforms, the source(s) from which their transcripts were cloned, and the length of the message (nt) and translated product (aa) are indicated to the right of the schema depicting the structural domains present in a given variant. FIG. 2C AND FIG. 2E depict the transcripts that encode the isoforms that lack the TM domain (i.e., isoforms lacking exon II). An in-frame initiation codon present at +101 in the exon Ib commences translation of an intact open reading frame but with a novel cytoplasmic tail (see FIGS. 1B and 3B). The splicing out of oxeon V (FIG. 2E; sDC-SIGN1B Type III) leads to the generation of a soluble variant with a novel C-terminus sequence (shaded box) of Exon VI. Similarly, splicing events in sDC-SIGN1 Type IV result in a novel C-terminus (FIG. 3D). †, and ††, denote the aa lengths of the DC-SIGN1B translated sequences that initiate translation at either +1 in exon Ia (41 aa) or +101 in exon Ib (varying lengths). [symbol] denotes skipping of the indicated exons/sequences. Because of splicing events in exons III-VI, these exons can be further subdivided (e.g. exon IIIA, IIIB etc.).

FIG. 3A. Generation of sDC-SIGN and DC-SIGN1B isoforms, and alignmnet of amino acid sequences of prototypic mDC-SIGN1A, sDC-SIGN1A, mDC-SIGN1B and DC-SIGN2. Amino acid sequences at the junctions of exon Ic and exon III generated by the splicing of the TM-encoding exon II.

FIG. 3B. Amino acid sequence of the N-terminal end of transcripts that contain exons Ia and Ib, i.e., DC-SIGN1B isoforms. The nucleotide sequence of exon Ia, Ib and the initial portion of exon Ic is shown. The open reading frame initiated at the Met (ATG) codon in exon Ia gives rise to a trauncated protein of 41 aa (MSD . . . PRLstop), terminating with a stop codon in exon 1b (+124-126). The open reading frame initiated from a start codon at +101-103 in Exon Ib to encodes DC-SIGN1B products, that except for the N-terminus MASACPGSDFTSIHS, are identical to the prototypic DC-SIGN1A isoforms.

FIG. 3C, FIG. 3D, AND FIG. 3E. Splicing patterns of exon II-lacking transcripts that encode sDC-SIGN1 isoforms with novel C-termini. Stop codons are boxed. The antisense orientation primer used for PCR amplification is underlined in FIG. 3E.

FIG. 4. Schema of alternative splicing events that lead to the generation of membrane-bound or soluble DC-SIGN1 gene products. Splicing event #1 links the end of exon Ia to the beginning of exon Ic and generates the previously described prototypic DC-SIGN1A message (mDC-SIGN1A Type I; FIG. 1A). Additional splicing events in htis primary mDC-SIGN1A mRNA generates exon II-retaining mDC-SIGN1A Types II-IV mRNAs. Splicing event #2 links the end of exon Ic to exon III generating the prototypic exon II-lacking sDC-SIGN1A message (sDC-SIGN1A Type I mRNA), and additional splicing events in this message leads to exon II-lacking sDC-SIGN1A Types II-IV mRNAs. In contrast, transcripts in which splicing event #1 does not occur generate the prototypic exon II-retaining mDC-SIGN1B message (mDC-SIGN1B Type I mRNA) and/or tDC-SIGN1B (exon Ia+partial exon Ib). Splicing event #2 in the mDC-SIGN1B primary transcript lins the end of exon Ic to exon III generating the prototypic exon II-lacking sDC-SIGN1B Type I mRNA, and additional splicing events in this message leads to exon II-lacking sDC-SIGN1B Types II-IV mRNAs. The structure of the translation products of the mRNAs generated by these splicing events are shown in FIG. 3. Exons and introns (not to scale) are designated by boxes and lines, respectively.

FIG. 5A. Molecular basis of the generation of membrane-bound and soluble DC-SIGN2 transcripts. The splicing patterns are determined by comparing the cDNAs cloned withthe genomic sequences of DC-SIGN2. The numbering system is based on the nucleotide sequence deposited under GenBank Accession Numbre AC008812 and the first nucleotide o the initiation Met codon of the prototypic mDC-SIGN2 (Type I) mRNA transcript is considered as +1. Topmost panel is a schematic illustration of the DC-SIGN2 gene. Horizontal lines are exons (I-VII) and dashed lines illustrate the splicing events that lead to the formation of the prototypic exon III-containing DC-SIGN2 mRNA transcript (mDC-SIGN2 Type I). Exon III encodes the TM domain (FIG. 6), and the exon III-retaining DC-SIGN2 mRNAs enclode membrane-bound or mDC-SIGN2 isoforms, whereas mRNAs that lack this TM-encoding exon III encode soluble or sDC-SIGN2 isoforms. Alternative splicing events that lead to the generation of DC-SIGN2 mRNAs that contain or lack the TM-encoding exon III can be recreated byo the various exonic sequences indicated; the startg and ending nucleotide numberof each exonic segmaent is separated by dots (e.g. join 1 . . . 46), and exoic segments are separated from each other by a comma (e.g. 1..A6, 127 . . . 210, 1919 . . . 2002). Asterisk indicates the stop codon used by most rn or sDC-SIGNtranscripts, whereasthe dsindkate the stop codon utilized by mDC-SIGN2 mRNAs Type V and VI at positions 5608561Q. Sequences coresponding to exon IVa were found only in the sDC-SIGN2 transcripts. Note that repeats 3, 4 & 5 cannot be disingushed from each other; hence the splice junctions for mDC-SIGN2 type VI and sDC-SIGN2 type I transcripts cannot be inferred.

FIG. 5B. DC-SIGN2 mRNA transcripts that use non-canonical splice donor and/or acceptor sites.

FIG. 5C and FIG. 5D. Alternative splicing events that lead to the generation of sDC-SIGN2 isoforms with novel C-termini. [symbol] denotes skipping of the indicated exons/sequences. Stop codons are boxed.

FIG. 5E. Amino acid sequences encoded by exon IVa, and alignment of the region bridging exon II and exon IVb in sDC-SIGN2 and mDC-SIGN2 isoforms.

FIG. 6. Structure and molecular diversity of membrane-bound and sohble DC-UGN2 gene products. Gene organization of DC-SIGN2. Boxes are exons (I-VIII) and dashed lines are introns (I-VII in black circles). Th nucleotide lengths of the introns are shown in parentheses. The first nucleotide of the initiation Met codon of the prototypic mDC-SIGN2 (Type I) mRNA transcript is considered as represents the 3′-untranslated region (UTR). The structure of the prototypic mDC-SIGN2 protein product is shown in FIG. 6B. Exons I, II, and a portion of exon III encode a short cytoplasmic (CYT; open boxes) domain; the transmembrance (TM; box with forward slash) domain is encoded by sequences in exon III. Exons IVb-VII encode the extracellular (EC) domain of the prototypic mDC-SIGN2 and this includes a short stretch of sequence just proximal to the repeats (box with horizontal lines), the seven full repeats and one half repeat (numbered boxes), and the lectin-binding domain (backward slash). The shaded box (2351-2372) represents the alternatively spliced exon IVa that is found only in those isoforms that lack the TM-encoding exon III. The Image Clone no. 240607 was a partial cDNA clone that contained exons V, VI, and VII. The alignment of the amino acid sequences of the DC-SIGN2 isoforms depicted in this figure is shown in FIG. 8.

FIG. 6B AND FIG. 6C. Schematic illustration of the molecular diversity and structures of DC-SIGN2 isoforms generated by alternative splicing events. FIG. 6B and FIG. 6C depict the transcripts that encode the isoforms that contain the TM domain (mDC-SIGN2 isoforms) and isoforms that lack the TM domain (sDC-SIGN2 isoforms), respectively. Amino acid differences among the isoforms, and the soure(s) from whrh their transcripts were cloned are indcated to the right of the schma depicting the stncral domains present in a given variant. Retention of intron IV leads to formation of a novel C-terminus in mDC-SIGN2 types II and IV and sDC-SIGN2 type II shaded box. Due to splicing out of exon VI, a novel C-terminus is predicted to form in mDC-SIGN2 types V and VI shaded box. The sDC-SGN2 isoforms exclusively contain a short hydrophobic stretch of amino acids, due to the presence of exon IVa [symbol] denotes skipping of the indicated exons/sequences.

FIG. 7. Colocalization of DC-SIGN1 (CD209), DC-SIGN2 (CD209L) and CD23 to within ˜85 kbp of chromosome 19p13.3. All three genes are subject to highly complex splicing events (Delespesse et al. 1991; Yokota et al. 1988; Yoshikawa et al 1999).

FIG. 8. Alignment of the predicted amino acid sequences of DC-SIGN2 mRNA isofoms. Type I mDC-SIGN2 is the prototypical DC-SIGN2 isoform that represents the membrane version of the protein. The sDC-SIGN2 isoforms lack the region encoded by Exon III that encodes the TM region. The sDC-SIGN2 isoforms also differ from mDC-SIGN2 isoforms by eight amino acids that are encoded by Exon IVa. Amino acid sequences that are encoded by distinct exonic regions are demarcated. The start of each repeat is indicated (R1-R8). The first “full repeat” and the eighth “half repeat” are overlined. The novel C-terminus present in mDC-SIGN2 Types III and IV and sDC-SIGN2 Type II is shown and the C-terminus present in mDC-SIGN2 Types V and VI is shown. Dots indicate identitya and dashes indicate deletions.

FIG. 9. sDC-SIGN blocks HIV-1 trans-infection of GHOST target cells expresssing CD4/CCR5. THP-1 cell line were transfected with mDC-SIGN1 prototype version and the trans-infection assay was done as described in FIG. 5. To block the interaction between transfected cells and target cells, the inventors used the sDC-SIGN1A type I recombinant protein described in FIG. 1. Lane 1) THP-1 untransfected cell line; Lane 2) THP-1 untransfected cell line+recombinant sDC-SIGN1 (20 ug/ml); Lane 3) THP-1 transfected cell line; Lane 4) THP-1 transfected cell line+recombinant sDC-SIGN1 (20 ug/ml). The recombinant sDC-SIGN blocks the ability of transfected cell lines to transfer virus to target cells (compare lanes 3 and 4).

FIG. 10. The efficiency of DC-SIGN1-mediated virus transfer was assessed in a coculivation assay. HeLa cells were transfected with DC-SIGN1 variants cloned in pcDNA/HisMaxTOPO Vector (Invitrogen) and stable clones were selected by using Zeocin. Transfected cell were incubated with 5 ngm of luciferase reporter virus ADA, and after 5 hours of incubation, the cells were washed extensively with fresh DMEM and coculivated with GHOST target cells. Two days later, the cells were lysed with a commercially available lysis buffer (Promega). Luciferase activity in 30 microliters of cell lysate was determined using a commercially available kit Untransfected Cell Line with target cells expressing CD4+CCR5. (3)Hela Cell Line Transfected with DC-SIGN1 Full Length (see FIG. 1B) with target cells expressing CD4. (4) Hela Cell Line Transfected with DC-SIGN1 Full Length (see FIG. 1B) with target cells expressing CD4+ccr5. (5) Hela Cell Line Transfected with mDC-SIGN1A type II (lacks six amino acids in the lectin-binding domain) with target cells expressing CD4 (see FIG. 1B). (6) Hela Cell Line Transfected with mDC-SIGN1A type II with target cells expressing CD4+ccr5, (7( Hela Cell Line Transfected with sDC-SIGN1A type II (see FIG. 1C; lacks exons Ic and II) with target cells expressing CD4, (8) Hela Cell Line Transfected with sDC-SIGN1A type II with target cells expressing CD4+ccr5.

FIG. 11A. gp120 & ICAM-3 Fluorescent Bead Adhesion Assay. Schema showing the preparation of ligand-coated beads. Ligand (gp120 or ICAM-3) coated beads were prepared as described previously by Geijtenbeek et al. (1999). Briefly, carboxylate-modified TransfFluor-Spheres (488/645 nM, 1.0 μM) wre coated with HIV-1 gp120 or ICAM-3 as follows. Streptavidin-coated beads were incubated with biotinylated F(ab′)2 fragment goat anti-mouse IgG followed by mouse-anti-gp120 mAb or biotinylated F(ab′)2 fragment goat anti-human Fc. The beads were then incubated with HIV-1gp120 or ICAM-3/Fc.

FIG. 11B. Adhesion assay demonstrating the ability of the antipeptide DC-SIGN1 antiserum in blocking gp120 and ICAM-3 adhesion to DC-SIGN1-transfectants. 1×10⁵ DC-SIGN1-transfected THP-1 cells were preincubated with competitor for 10 min at room temperature. Ligand-coated fluorescent beads (20 beads) were added, and the suspension was incubated for 30 min at 37° C. Adhesion was determined by measuring the percentage of cells, which have bound fluorescent beads, by fPw cytotrAy using the FACS calibur. One of three represenative results is shown. The number of the cells binding to the fluorescent beads is shown as a percentage in the boxes. The antipeptide antiserum decreased the adherence of DC-SIGN1 transfectants to gp120 beads (top panel) and to ICAM-3 (bottom panel). Unlabeled gp120 completely inhibited the binding of ICAM-3 to DC-SIGN1 transfectants.

FlG. 12A. Soluble DC-SIGN2 isoforms from BeWo Cell Line (Choriocarcinoma). RNA was isolated by using a Trizol reagent from Gibco BRL, total RNA was retrotranscribed and amplified by using specific priers for DC-SIGN2. After nested PCR, the product was cloned in pCRII TOPO vector and sequenced. Prototype DC-SIGN2 mRNA. The exons I and II encode the cytoplasmic domain, Exon II encodes the transmembrane domain, exon IV the repeats region and exons V, VI and VII encodes the Lectin Binding Domain.

FlG. 12B. Description of the isoforms at RNA level. BeWo-I lacks Exons II and III, BeWo-II lacks the transmembrane domain and Exon VI, which change the open reading frame and create a novel C-terminus as described. BeWO-III lacks exons II, III and VI, and BeWo-IV lacks exon III and repeat 5. This is a description of a new set of DC-SIGN2 variants lacking the transmembrane domain (Exon III) from a Choriocarinoma cell line. These new variants do not contain intron IV or exon IVa, which create a new set of variants with potential effect in HIV pathogenesis because its predicted domains.

FIG. 13. Polymorphisms in the DC-SIGN1 gene: Nucleotide sequence alignment of DC-SIGN1 gene sequences of two DC-SIGN1 alleles. Dots are gaps in the nucleotide sequence and dashes are deletions. Nucleotide differences are indicated in red and their position relative to the ATG start codon in exon Ia is numbered.

FIG. 14 DC-SIGN2 alternative splicing variants expression in Choriocarcinoma Cell Lines and Placenta. Soluble- and Membrane-associated isoforms of DC-SIGN2 are the two broad classification, however splicing out of exons 2 and 6, as well as intron 4 retention are common alternative splicing variations. A high variation in the repeats are was also seen. Some of this last variationmay be partially explained for differences in the genomic composition since a wide variation in the amount of repeat has been reported. The range of variants is wider tat what was reported previously. Also the donor-acepbr site seems to be dhsen randomly, since no patterns arise from the discovered arrays and the combinations seems unlimited. More that 1000 dfrrnt isotrs could be produced in placental tissues that cannot be explain by differences in the genomic sequence.

FIG. 15 DC-SIGN2 Levels in plcenta lysates from different individuals. Sandwich EUSA was conducted using a polclonal antibody for capture and a monoclonal for detection. There are marked differmes in the levels of DC-SIGN2 protein being produced among different placentas.

FIG. 16 Fractions from different sites in the same placenta were analyzed for expression in an ELISA assay for DC-SIGN1. There were subtle differences among distinct areas of the same placenta, but indMdual placentasshowed a trend in its levels.

FIG. 17 Fractions from different sites in the same placenta were analyzed for expression in an ELISA assay for DGSIGN2 There were subtle differences among distinct areas of the same placenta.

FIG. 18 DC-SIGN alignment showing the similarity among all three molecules (gray mtters). The armino acids recognized as important for ligand binding are labeled with an asterisk. DC-SlGN3 sequence is as presicted in XM 064898. Translaton starts at amino acid 59. All three molecules have the conserved motif EPN which is a hallmark forthe lectin binding mannose residues.

### DESCRIPTION OF ILLUSTRATIVE EMBODIMENTS

A. DC-SIGN Activities and DC-SIGN Isoforms

Expression in dendritic cells (DCs) of DC-SIGN, a type II membrane protein with a Cape lectin ectodomain, is thought to play an important role in establishing the initial contact between DCs and resting T cells. DC-SIGN is also a unique type of human immunodeficency virus-1 (HIV-1) attachment factor and promotes efficient infection in tens of cells that express CD4 and chemokine receptors. The inventors have identified other genes, designated here as DC-SIGN2 and DC-SIGN 3, that exhibit high sequence homology with DC-SIGN. Here the inventors demonstrate that alternative splicing of DC-SIGN1 (original version), D-SIGN2 and DCSIGN3 pre-mRNA generates a large repertoire of DC-SIGN-like transcripts that encode membrane assoated and soluble isoforms.
1. DC-SIGN1 and DC-SIGN2

The range of DC-SIGN1 mRNA exprsion was signiicanty broader than preusly repoted and included THP-1 monocytic cells, placenta, and peripheral blood mononuclear cells (PBMCs), and there was cell rntuntion/cvatior-induced diferences in mRNA expression levels. Immunostaining of term placenta with a DC-SIGN1-specific antiserum showed that DC-SIGN1 is expressed on endothelial cells and CC chemokine receptor 5 (CCR5)-positive macroae-ike cells inte villi.

DC-SIGN2 mRNA expression was high in the placenta and not detectable in PBMCS, In DCs, the expression of DClSIGN2 transcripts was significantly lower than that of DC-SIGN1.

Notably, there was significant inter-individual heterogeneity in the repertoire of DC-SIGN1 and DC-SIGN2 transcripts expressed. The genes for DC-SIGN1, DC-SIGN2, and CD23, another Type II lectin, colocalize to an ˜85 kilobase pair region on chromosome 19p13.3, forming a cluster of related genes that undergo highly conpa atemaraive splicing events. The mnoleclar diversity of DC-SIGN-1 and -2 is reminiscent of that oberved for O n other adheie cell surface proteins invoed in cele connectivity. The generation of this large collection of polymorphic cell surface and soluble varints that exhibit inter4ndividual variation in expression levels has important implications for the pathogenesis of HIV-1 infection, as well as for the molecular code required to establish complex interactions between antigen-presenting cells and T cells, i.e. the immti4bgical synapse.

Because of the apparent role of DC-SIGN in HIV-1 pathogenesis and DC-T cell interac , the inventors hyphesed that mutations influencing the gene expression of this molecule and/or its interactions with HIM1 gp120 or ICAM-3 could have an impact on the pathogenesis of HIV-1 infection. As a first step in testing this hypothesis, the inverbs elucidated the gene and mRNA structure as well as the expression pattern of DC-SIGN.

In this study, the inventors identiied another highly homologous gene designated here as DC-SIGN2 and made the surprising observation that plasticity of the DC-SIGN1 (orginal version) and DC-SGN2 gene generates a wide repertoire of DC-SIGN1 and -2 transcripts. Interesingly, in addition to DC-SIGN1 (CD209) and DC IGN2 (CD209L), the low affinity immunoglobulin Fc eceptor (CD23) also maps to chromosome 19p13.3 forming a cluster of highly a cluster of highly relted genes that al undergo complex alternative splicing events (Delespeese et al., 1991; Suter et al., 1987). In contrast to previous to previous reports (Geijtenbeek et al., 2000a; Geijtenbeek et al., 2000b), the inventors show that the mRNA expression of DC-SIGN1 (original version) is not restricted to DCs but is broader and inclues placenta, PBMCs, and THP-1 monocytes. The inventors also found that there was cell raatunooio and/or ac n-ationinckt differences in DC-SIGN1 mRNA expession levels. By using a DC-SIGN1-specific antiserum, the invenors bund that DC-SIGN1 was epessed on the endothelial cells of the placental vascular channels and also co-expressed with CCR5 in the placental matrophages. Abundant DC- SIGN2 mRNA expression was detected in the placenta, but significantly less in THP-1 monocytc cells and DCs, whereas mRNA expression in resting or activated PBMCs was not detected. Notably, there was interAividual vanation in the exproosion levels as well as the repertoires of DC-SIGN1 and DC-SIGN2 transcripts expressed. While this paper was being prepared for submission and was in review, Soilieux et al. (2000) described a DC-SIGN homologue designated as DC-SIGNR that is identical to the prototypic membrane associated DC-SIGN2 described herein, and Pohlmann et al. (Pohlmann et al., 2001) showed that DC-SIGNR binds to HIV/SIV and activates infection in trans. Thus, the discovery of an extensive repertoire of DC-SIGN-1 and -2 transcripts with variable expression levels may have important implications for the pathogenesis of HIV-1 infection and the generation of T cell immune responses.

DCs are thought to act as Trojan horses, capturing HIV in the mucosal surfaces for transport to the T cell areas of draining lymphoid tissues. The proficiency of DCs in interacting with T cells makes them prime candidates for enhancing viral infection. Recent reports indicate that DC-SIGN, a surface receptor with high expression in DCs, may play an important role in DC-T cell as well as DC-HIV interactions (Geijtenbeek et al., 2000a; Geijtenbeek et al., 2000b). The inventors have significantly extended these initial reports by (i) discovering that complex alternative splicing events in DC-SIGN (designated here as DC-SIGN1) pre-mRNA generates a wide repertoire of DC-SIGN1 transcripts. These DC-SIGN1 transcripts encode both membrane-associated (mDC-SIGN1-A or -B) as well as soluble (sDC-SIGN1-A or -B) isoforms with varied intracellular and/or extracellular ligand (gp120/ICAM-3) binding domains. (ii) The inventors have identified another highly homologous gene designated here as DC-SIGN2. Similar to DC-SIGN1, alternative splicing of DC-SIGN2 pre-mRNA also generates a wide repertoire of DC-SIGN2 transcripts that encode membrance-associated and soluble isoforms. (iii) Interestingly, in addition to DC-SIGN1 (original version) and DC-SIGN2, the inventors found that the low affinity immunoglobulin Fc receptor (CD23) also maps to chromosome 19p13.3, forming a cluster of highly related genes that all undergo highly complex alternative splicing events (Delespesse et al, 1991; Suter et al., 1987). (iv) In contrast to previous reports (Geijtenbeek et al., 2000a; Geijtenbeek et al., 2000b), the inventors found that DC-SIGN1 mRNA expression is not restricted to DCs but is significantly broader and includes THP-1 monocytic cells, resting CD14+ monocytes, PBMCs, and placenta. Immunostaining with a DC-SIGN1-specific antibody indicated that DC-SIGN1 is expressed on placental endothelium as well as on CCR5+ cells. The distribution of these CCR5+DC-SIGN1+ cells is consistent with that of placental macrophages. (v) DC-SIGN2 transcripts were also detected in placenta but not in PBMCs. In contrast to DC-SIGN1, expression of DC-SIGN2 mRNA in DCs and THP-1 monocylic cells was significantly lower. (vi) Notably, the inventors found that there was inter-individual variation in the repertoire of DC-SIGN1 and DC-SIGN2 transcripts expressed and that there were cell maturation stage and/or activation state differences in the expression levels of DC-SIGN1 mRNA.

Alternative splicing of the precursor for mRNA (pre-mRNA) is a powerful and versatile regulatory mechanism utilized by higher eukaryotes for generating functionally different proteins from the same gene and accounts for a considerable proportion of proteonic complexity (Lopez, 1998; Black, 2000; Smith and Valcarcel, 2000). Indeed, there are remarkable examples of hundreds and even thousands of functionally distinct mRNAs and proteins being produced from a single gene. In the human genome, such protein-rich genes include neurexins (Ullrich et al., 1995; Missler and Sudhof, 1998), n-cadherins (Kohmura et al., 1998; Shapiro and Colman, 1999; Uemura, 1998; Wu and Maniatis, 1999), calcium-activated potassium channels (Black, 2000; Adelman et al., 1992), and others (Black, 2000; Schmucker et al., 2000; de Melker and Sonnenberg, 1999).

Alternative splicing is often tightly regulated in a cell type- or developmental stage-specific manner. Coordinated changes in alternative splicing patterns of multiple pre-mRNAs are an integral component of gene expression programs, like those involved in nervous system differentiation (Grabowski, 1998) and apoptotic cell death (Jiang and Wu, 1999). Similar programs are also likely to exist during T cell and DC differentiation (Marrack et al., 2000; Walker and Rigley, 2000; Dietz et al., 2000). In addition to cellular differentiation, the pattern of splicing can be influenced by the activation of particular signaling pathways (Fichter et al., 1997; Metheny and Skuse, 1996; Eissa et al., 1996; Mackay et al., 1994; Sharp, 1994; Roca et al., 1998; Luo et al., 1998). Notably, in the studies the inventors found that the expression pattern of DC-SIGN1 transcripts may depend, in part, on the cell maturation/activation state.

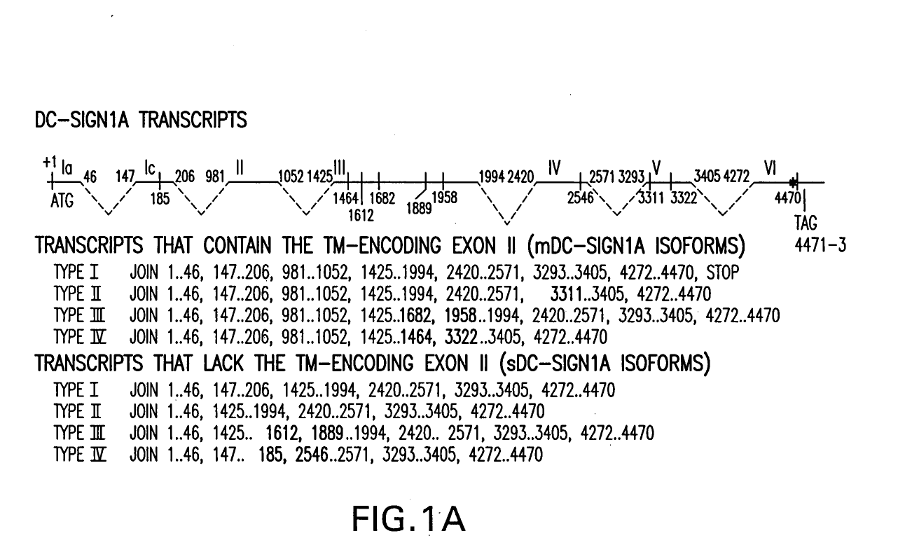

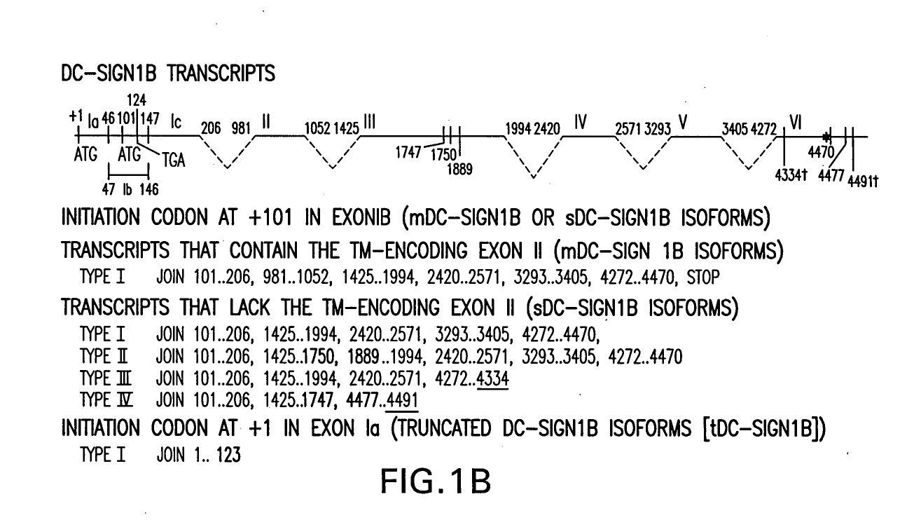

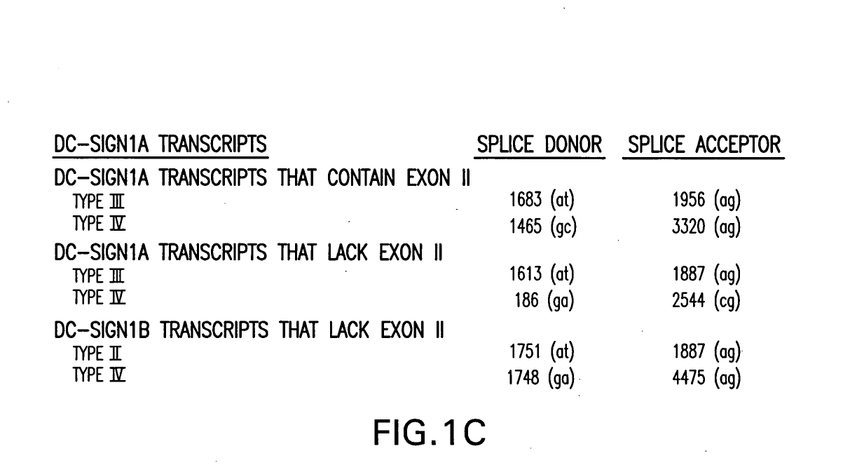

It is known that alternative splicing can generate mRNA structures that can take many different forms (Lopez, 1998; Black, 2000; Smith and Valcarcel, 2000). Exons ca be spliced into mRNA or skipped. Introns that are normally excised can be retained in the mRNA. The positions of either 5′ or 3′ splice sites can shift to make exons longer or shorter. In addition to these changes in splicing, alterations in transcriptional start site or polyadenylation site also allow the production of multiple mRNAs from a single gene. It is remarkable that nearly all of these variations in mRNA structure were observed in DC-SIGN1 and DC-SIGN2 transcripts (FIGS. 1-6).

A emerging paradigm is the observation that proteins involved in cell-cell contact oriocognition often exhibit a high degree of molecuar diversity. Examples include genes for cadherins, cadherin-related neuonal receptors, dwactory receptors, and neueons in the nervous system (Ulrich et al., 1995; Kohmura et al., 1998; Shapiro and Colman, 1999; Uemwa, 1998; Wu and Maniatis, 1999; Wang et al., 1998) and for immunoglobulin and T cell receptor genes in the immune system (Cook ard Tomlirson; Wdbams etal, 1996; Davis and Bjorkman, 1988). In this context, it is notable that DC-SIGN1-mecriated binding of DCs to lC.M3 on resting T cells is thought to be a key initial adhesion step in the multi-step process that leads to the formation of the immunological synas and the activation of resting T cells (Geijtenbeek et al., 2000a; 2000b). Thus, DC-SIGN1 (and potentialDy SIGN2) dam s the generality of the features found in certain other genes involved in cell-cell adhesion/recognition. These common features inclde etensive alternatve spcidng events, cell type- and activation-specific expression, and a similar domain structwre with dinct paterrsshad ed and divergent sequences.

The inventors have demonstrated the genomic basis for the generation of not orey several membrassociated but also potentially soluble forms of DC-SIGN1, DC-SIGN2 and DC-SIGN3. Furthermore, the studies suggest that the expression levels of DC-SIGN1 scripts that lack the TM-coding exon are not minor variants of the overall pool f DC SIGN1 mRNAs. Remarkably, the skipping of the TM-coding exon is observed in several type II membrane proteins that belong tote C-tpe anirmal lectin family (Yokota et al., 1988; Yoshikawa et al., 1999; Gordon, 1994; Ying et al., 1995; Bocek et al., 1997), suggest that this is an evomumnanIy conserved property.

Because DC-SIGN-1 and -2 lack a leader sequence, it is not dear w bher loss of the inphdi TMdng exoni would limit the ability of these molecules to traverse across the endoplasmic reticulum membrane, remft in their retention in the cytopbsi However, there are examples among the lectin family wherein molecules lacking the secretory signal are extealized by mechanis other than the classical secretory pathway (Cooper and Barondes, 1990). Notably, ceran other cytoplasmic proteins bdn a signal sequence are externalized and function extracellulary. These include IL-1 (March et al., 1985), Iboblast growth factor (Abraham et al, 1986; Jaye et al., 1986), and others (Clinton et al., 1989 Goodall et al., 1986). Aftematively, these TMacking DC-SIGN isofomis may functon as intracellular molecules. For example, the invariant or chain, another type 11 meembrm protein, is responsible for targeting the Class II dimers to the endocytic pathway that influences the delivery of antigens (Neeqes and P*egh, 1992).

The inventors found that the mRNA expression pattern for DC-SIGN1 was broader On reported prevbusly (Geftnbeek et al., 2000a, 2000b). For example, the inventors cloned the transcripts of DC IGN1 from THP-1 cells and PBMCs. Expression of DC-SIGN1 mRNA, albeit low was detected in resting PBMCs. In contrast, in PBMCs stimulated with PHAor CD31CD28 (stimuli of the T cell receptor) there was an increase in DC-SIGN1 mRNA expression. In studies not shown, DG-SIGNI MRNA expression in PBMCs also increased significantly after stimulation with PMA and ionomycin, a calcium ionophore; this frm of stimulation is known to activate the PKC pathway in T cells by bypassing the T-cell receptor. In ongoing studies the inventors ae inves the pre cell types in resting as well as in PHA-, CD3/C28-, and PMA/ionomycin-activated PBMC cultures that expess DC-SIGN1 MRNA. I is diffcult at the present moment to reconcile the diffrences between the findings and those of Geijtenbeek et al. (2000a; 2000b) whose studies indicated that the expression of DC-SIGN1 is DC-specific. By using a PCR-based strategy type found no mRNA expression for DC-SIGN1 on THP-1 cells, granulocytes, PBMCs activated for 2 days with PHA and IL-27 or per blood leukocytes (Geijtenbeek et al., 2000a; 2000b). The reasons for this discrepancy remain unclear but could be related to differene in PCR conditions or primer design. The inventors are currently in the process of generating monoclonal antibodies to determine wheltt thee is a discordance between the levels of DC-SIGN1 mRNA and protein expression. Notably, there are several examples of bssue or cet typespecific regulation of translation, including that for IL-2 (Garcia-Sanz and Lenig, 1996; Corradi et al., 1997; Rao and Howells, 1993; Ruan et al., 1994; Hill and Morris, 1993; Imataka et al., 1994; Bloom and Beavo, 1996; Garcia-Sanz et al., 1998; Mikulits et al., 2000).

The inventors found that the genes for DC-SIGN1 (CD209), DC-SIGN2 (CD29L), and CD23 cbcale to an -85kb regon of chromosome 19p13.3. Alternative splicing events in CD23 generates several transcripts including two isoforms (FcRIla/CD23a and FcRIb/CD23b) that differ only at the N-terminal cytoplasmic region (Delespesse et al., 1991; Yokota et al., 1988;Yoshikawa et al., 1999). Interestingly, FcRIIa (CD23a) and FcRIIb (CD23b) exhibit differences in their tissue expsssion, and IL4 differentially regulates their expression (Yokota et al., 1988; Kikutani et al., 1989). These two CD23 isoforms also have differential functions in allergic reactions, immunity to parasitic infections, and B cell development (Yokota et al., 1988; Kikutani et al., 1989). As a corollary, the inventors found that alternative splicing of DC-SIGN1 pre-mRNA also leads to the generation of transcripts Ihat encode distinct Nerminal regions (DC SIGN1-A and -B) and that IL-4 differentialy regulates the expession of DC-SIGN1 in DCs. There is growing eyidence that lectins, including CD23, can serve as cell surface transducers of signals from the outside to the sie of the cell (Sanrho eta(, 2000; Hebert, 2000); in this context, the inventors are currently investigating whether DC-SIGN1-A and iobrms actvate distinct intracelular signaling pathways.

The biological properties of this large repertoire of DC-SIGN1 and -2 issormswih respect to their roles in HIV pathogenesis and DC-T cell interactions remains unknown. Changes in splicing have been shon to determine the igand binding of growth factor receptors and cell adhesion molecules (Lopez, 1998; Smith and Valcacel, 2000). The mDC-SIGN1 and mDC-SIGN2 isoforms with varied extracellular domains may bind ligands, including gp120, with varied avidity. Furthermore, in addition to ICAM-3, this extensive array of membrane-associated DC-SIGN1s (and potentially mDC-SIGN2s) may mediate cell-cell contact via interactions with a larger number of specific ligands or adhesion molecules of different protein families. Studies a currenty underway to determine whether, similar to the findings in other gene systems, an alternative splice variant of DC-SIGN1 or -2 cross-regulates or antagonizes the biological activities of the other isoforms (Jiang and Wu, 1999; Cote et al., 2000; Arinobu et al., 1999; Tsytsikov et al., 1996; Boise et al., 1993; Wang et al., 1994; Shaham and Horvitz, 1996; Nakabeppu and Nathans, 1991; Tone et al., 2001). For example, an alternatively spliced isoform of CD40 influences the function of the prototypic fultlength CD40 isoform (lbne etal, 2001). An intriguing possibility is that the DC-SIGN-1 and -2 isoforms backing the tarsmembrane domain if secreted may act as natural competitive inhibitors of D SIGN/ICAM-3/HIV binding interactions in vivo, or alternatively, they may function in regulatig the expresion of the membrane forms of DC-SIGN. Furthermore, lectin-binding domains can oligomerize (Drickamer, 1999; Weis et al., 1998; Weis and Drickamer, 1996; Drickamer, 1995; Drikamer and Taylor, 1993), and potentially this oligorzaion amo the varied membrane fomis of DC-SIGN1 or between DC-SIGN1 and DC-SIGN2 isoforms in cell types in which they are m rnay firter increase the repertoie and specificity of DC-SIGN-like surface proteins available for mediating cell-cell contact.

The prototypic membrane-associated DC-SIGN1 (mDC-SIGN1 Type I) and DC-SIGN2 (mDC-SIGN2 Type I) isoforms have been shown to mediate gp120 adhesivity and potentiate in trans the infection of T lymphobytes by HIV (Geijtenbeek et al., 2000a; 2000b; Pohlmann et al., 2001). By mRNA expression studies and immunostaining, expression for DC-SIGN1 was detected in both placental endothelial cells and CCR5-expressing cells in which distribution was conistnt with pacenta macrophages (Hofbauer cels), a cell type that can support HIV infection (Newell, 1998). The inventors also detected DC-SIGN2 tanscripts in the lacenta; and while this manuscript was in review, using a DC-SlGN2-specific antiserum, Pohlmann et al., (2001) docwneted expreson for DC-SIGN2 in the placental endothelium but not macrophages. The expression of both DC-SIGN1 and DC-SIGN2 in the placenta has important implicatons for vertical transmission of HIV-1. However, pertinent to the search for geneti determinants that account for the significant inter-individual variability in susceptibility to HIV infection, the studies indcate that DCSIG 1 and DC-SIGN2 gene expression in the placenta and other cell types may be highly variable. The inventors examined a large pnel of placenta samples, and found inter- individual variation with respect to both the levels of expression as well as the repertoire of transcripts expressed. Notabty, in some instances, the inventors were unable to detect expression for the prototypic mDC-SIGN2 transcripts in placenta, and transcripts that contained intron IV appeared to be more abundant than the prototypic isoform. Convabl, inter-indicdual variation in the generation of DC-SIGN isoforms could account, in part, for host differences in susceptibility to HIV-1 infection, especially vertical transmission.

While searching for polymorphisms in the gene for DC-SIGN1, the inventors identited anoter homotogous gene designated here as DC-SIGN2 that recently has been shown to serve also as an HIV attachment facor. Notably, the inventors found that alternative splicing of DC-SIGN1 and DC-SIGN2 generates a wide array of transcripts that encode both membraneassocated and soluble isoforms. Determining the functional properties of this extensive repertoire of DC-SIGNI and DC SIGN2 isofrrns in vivo is lrkely to pose a daunting task, and in this respect it will be important to develop reagents that can discrinte beten the differnt isolbrrns. In addition, the inter-individual heterogeneity in DC-SIGN expression, especially DC-SIGN2 in plaenta, inreduces an unartdipated degree of complexity with regard to dissecting the determinants of HIV susceptibility. Nevertheless, this p a of DC-SIGN like molecules wil serve as a powerful tool to probe HIV-host cell interactions as well as DC-T cell iand as a potential target for a novel reans to block these interactions. Based on the striking parallels between DC-SIGN-1 and -2 and other altenively spliced type 11 membrane proteins such as CD23, the inventors believe that the diverse DC-SIGN isoforms have pleiotropic acdivies and that they may interact with additional, as yet undiscovered molecules.
2. DC-SIGN3

DC-SIGN3 is a protein that contains a pulative conserved lectin domain from the arnino acids 49-172 No hydrophobic pulative transmembrane domains were predicted. Some Web based programs (SignalP V1.1 World Wide VWeb Server) suggest that this protein could undergo changes leading to the secretion pathway. It has a broad expression pattern and the inventors have observed mRNA expression in the cerebellum, spleen and PBMCs. The inventors are currently extending their research towards a complete characterization of the expression of DC-SIGN3 to other tissues and cell lines.

The initial three-dimensional modeling predicts that DC-SIGN3 contains all the touch-points necessary for the binding to gp120, the envelope protein on the surface of the HIV-1. The inventors have determined that thisfing could lead to a more complete understanding of the AIDS immuno-pathogensis as well as new therpeutic targets against HIV-1 irfion

DC-SIGN-1 is predicted to bind mannose residues in a calcium-dependent manner through its C4ype lectin (CTL) and CTL-like domains. Many animal C-type lectins are involved in extracellular matrix orga n and endo . These lctins also complement activation and mediate pathogen recognition and cell-cell interactions. Such lectins, as serum mannosebinding prons, pulmonary surfactant proteins, and macrophage cell-surface mannose receptors, bind terminal residues chaacterstic of bacterial and fungal cell surfaces. They may bind a variety of carbohydrate ligands ircfiding mannose, N cetlglsarnine, galactose, N-acetylgalactosamine and fucose. Several CTLs bind to protein 6s, and only some of these binding interacons are Ca2+-dependent; such CTLs include Coagulation Factors IX/X (IX/X) and Von Wilebrad Facbr (VWF) binding proteins, and CTL-like natural killer and hematopoietic cell receptors. CTLs such as pancreatic protein hne and some type II antifreeze glycoproteins function in a Ca2+-independent manner to bind inorganic surfaces. CTL dorais associate with each other through several difnt surfaces to form dimers, trimers, or tetramers, from which ligand-binding sites project in a variety of diferent orientations. In some members (IX/X and VWF binding proteins), a loop extends to the adjoining domain to form a loop-swapped dimer. A similar conformation is seen in the macrophage mannose receptor CRD-4's putative non-sugar bound form of the domain in the acid environment of the endosome.

B. Diseases and Methods of Treatment
1. HIV and Other Viruses

The dissemination of HIV-1 and establishment of infection within an individual involves the transfer of virus frorn musal sdtes of infection to T cell zones in seconday lymphoid organs. How this happens is not precisely know. IHowever, there is growing support for the notion that dendritic cells (DCs) present within the mucosal sites may play a central role in thispcess (Banchereau and Steinman, 1998; Knight and Patterson, 1997; Weissman and Fauci, 1997; Cameron et al., 1992, Caraan et al., 1994; Pope et al., 1994; Pope et al., 1995; Canon et al., 1996; Granelli-Piperro et al., 1996; Graneli-Popeno eta(, 199; Granelli-Piperno et al., 1995; Zailseva et al., 1997; Reece et al., 1998; Weissman et al., 1995; Weissman et al., 1995; Canque et al., 1999, Blauvelt et al., 1997; Cimarelli et al., 1994; Pinchuk et al., 1994; Tsunetsugu-Yokota et al., 1995; Tsunetsugu-Yokota et al., 1997; Frank et al., 199, Kacani et al., 1998). The normal function of these DCs is to survey mucosal surfaces for antigens (Ags), captre Ag, c captured proteins into immunogenic peptides, emigrate from tissues to paracortex of draining lymph nodes, and present pepiws in the context of MHC molecules to T cells (Banchereau and Steinman, 1998). It is now generally berieved that HIV subverts this nomal trafficking Prooss to gain etry into lymph nodes and to CD4+ T cells. There is also evidence dernonsrtng that producthve ibta7 of DCs and the ability of DCs to capture virus with subsequent transmission to T cells is medated through two separae pathways (Granelli-Piperno et al., 1999; BlauveH e tal, 1997) and reviewed in (Weissman and Fauci, 1997; Steinman, 2000). Thus, strategies designed to block mucosal transmisson of HIV will require a clear understanding of the molecular determinants of not only virus infeclion, but also virus capture by DCs.

Two recent reports by Geijtenbeek et al demortrated that a mannose-binding, Ctype leclin design as DC-SIGN (DC-specific, ICAM-3 gabbing, nonintegrin) may play a key role in HIV pathogenesis (Geijtenbeek et al., 2000a; Geijtenbeek et al., 2000b). First, by binding to ICAM-3 expressed on T cells, DC-SIGN is thought to facilitate the initial interaction between DCs and naive T cells (Geijtenbeek et al., 2000a; 2000b), setting the stage for subsequent c a events that lead to Ag rocognifion and the formnation of a contact zone termed the immunological synapse (Steinman, 2000; Anton van der Merwe, 2000). Second, HWlf may exploit DC-SIGN for its transport via DCs from mucosal sfaees to secondary lymphoid a rich in activated mery CD4+ T cells that express CC chemokine receptor 5 (CCR5). Unlike CCR5, the major receptor for HIV-1 cell entry (Berger etal, 1999), DC-SIGN is not a co- receptor for viral entry. Geijtenbeek et al confirmed an earlier observatin that DC-SIGN is an HIV-1 envelope (gp120)-binding lectin (Curtis e tal., 1992), and extended significantly this finding by showing thati promotes efficient infection in cells that express CD4 and chemokine receptors (Gertenbeek etal, 2000a; 2000b; Berger et al., 1999). This delivery and subsequent transmisson of HIV in a DC-SIGN-dependent manner to viral replication-permissive T cells is thought to play a major role in viral ion, especialy at low concentrations of HIV (Geijtenbeek, 2000a; 2000b).
i. In Vivo Infection and In Vitro Infectability of DCs With HIV

The susceptibility and nature of HIV-1 infection in DCs in the peripheral blood is controversial (Weissnian etal, 1995; Thomas and Lipsky, 1994; Patterson and Knight, 1987), and reviewed in (Weissman and Fauci, 1997; Blauvelt et al., 1997). Part of this controversy relates to the fact that (a) there are multiple populations of cells with DC morphology in the peripheral blood; (b) experimental methods to isolate DCs differ from study-to-study; (c) small numbers of contaminating T cells and monocytes/macrophages in DC populations may produce misleading results; and (d) different methods have been used to define productive infection (e.g. transmission election microscopy).

In tissues in which DCs reside for purposes of surveying Ags or in lympoid organs where Uhy adivate T cets, DCs are not highly or productively infected. However, that is not to say that DCs do not play a role initiatirng or ng Hinfction or that they may not become productively infected outside of the skin or lymphoid organ.
ii. Role of DCs in the Initiation of HIV Infection.

DCs are infected in vivo, albeit at low levels compared to CD4+ T cells. However, DCs have a role in HIV infection that is independent of direct DC infection, dysfunction, and depletion. One of the main pathologic processes in which the DC is involvee appears to be the initiation of HIV infection following exposure of vhu& A model has been proposed Wherein HIV enters the mucous membrane and interacts with tissue DCs, resulting in the binding of virus to the DC with or without irkion. DCs-then migrate to the paracortical region of the draining lymphoid tissue where virus is delivered to CD4+T cells that then become infected, and replication and spread of virus occurs. In vivo experiments in both mice (Masurier et al., 1998) and monkeys (Spira et al., 1996; Soto-Ramirez et al., 1996; Hu et al., 2000; Miller et al., 1993) support the hypothesis that DCs play a critical role in the crsaniatuon of vinus from the genital tract to lymphoid organs in the first 24 hours after HIV exposure.

It is notable that the primary site of HIV replication is in the paracortical region, the sae region to which DCs migrate to interact with T cells, initiating primary immune responses and propagale ongoing responses. Indeed, one of the best milieus for infection by HIV-1 is conjugates of lymphocytes and DCs (Cameron et al., 1992; Cameron et al., 1994; Pope et al., 1994; Pope et al., 1995; Granelli-Piperno et al., 1999; Zaitseva et al., 1997; Weissman et al., 1995; Weissman et al., 1995; Blauvelt et al., 1997; Pinchuk et al., 1994; Tsunetsugu-Yokota et al., 1995; Hadik et al., 1999; Spira et al., 1996; Kohmura et al., 1998; Berger et al., 1992; Pope et al., 1995; Ayehunie et al., 1995; Frankel et al., 1996). This DC-T cell microenvironment provides an -xlohe site for HIV repliocton, and the degree of infection appears to be related to the degree of T cell activation. There thus appear to be two pathways by which HIV interacts with DCs, both of which may occur simultaneously and independently of one another (Table 1; adapted in part from (Blauvelt et al., 1997)). One pathay leas to productive infecton and is HIV ccxeceptor and CD4-dependent and requires proligeratuon of DCs, whereas the abily of DCs to capture HIV is independent of HIV binding to CD4koreceptors, HIV reverse tcription and DC proliferation, but dependent on DC-SIGN expression (Geijtenbeek et al., 2000).

HIV enters the mucous membrane (sexual) or skin (needle stick) and binds to or infects tissue DCs. Upon recenving the appropriate signals, DCs traffic via the afferent lymphatics, entering lynp! od tissues thoough the subcapsular sinus and traverse toard the T cell area in the paracortical regions. Wdihin the paracortical region, the DC interacis with and CD4+T cells, leading to productive infection and spread of virus. DC-SIGN1 is expressed at high levels in DCs & binds infb vions via gp120 (Geijtenbeek et al., 2000a; 2000b). DC-SIGN captures both X4 and R5 tropi viruses and pesents these vm es to either CXCR4 or CCR5 expressing CD4+ T cells.

The role of DC-SIGN1 in this model has not been completely elucidated. However the cfingsof Curtis etal (1992) and more recently those of Geijtenbeek et al. (2000a; 2000b) would implicate an important role for this niol in virus capture, transport to lymphoid organs, and transfer of virus to T cells. Notably, Geitenbeek et al (2000al 2000b; Steinmnn 2000) demonstrated that DC-SIGN expressing cells can retain attached virions in an infectious state for several days and trans#tlm to replication-permisslve T cells. Indeed at low virus titer, infection of CD4/CCR5expressing cells was not detected without the help of DC-IGN in t Sexual transmission of HIV-1 presumably requires a means for small amounts of virus at mucos siles of inocdon to gain access tocells that are permissive for viral infection.

Taken together, the importance of the trafficking pathway of DCs to lymphoid ues in HIV patogenesis as well as DCT cel conjugates in the amplification process of HIV infection would suggest that in addition to blocking HIV entry, the targets for anSHIV strategies should include (a) viral binding to DC; (b) transfer of HIV to T cels; and (c) initation of events leading to a productive infection. Although blockade of DC-mediated initiation of T cell infection may be an imporat strategy for the treatment of HIV disease (Pinchuk et al., 1994; Tsunetsugu-Yokota, 1995), and can be accomplished by b n cmtnulatory Woleand interactions (e.g. wih mabs against CD4, ICAM-1, LFA-1, LFA-3, CD40, and CDBO), such a strategy would be impracical because this approach would also inhibit nominal immune functions. A more practical measure for blocking DC-mediated HIV infecion of T ols may be to develop agents that specifically block capture of HIV by DCs. An example of this could be anti-DC-SIGN1 agents thy specifcaty block capture of Hi by DCs, i.e. block gp120-DC-SIGN binding without disturbing ICAM-3-DC-SIGNI interactions (Geitenbeek eta( 2000a, 2000b).
2. Combinational Therapy

It is also contemplated that polypeptide and/or nucleic acid composition of the DC-SIGN1, 2 or 3 isoforms in combination with other anti-viral agents would be effective in treatng viral infectios such as HIV and ebola viral infections.

For example, not only do DC-SIGN compositons provide an effective therapy for HIV on their own, they also improve the efficacy of other traditional anti-viral compounds as well. In particular, the DC-SIGN compositions can be used in combination with known compounds effective in the treatment of HIV infection. These known compounds include, but are not limited to nudeoside reverse transcriptase inhibitors (NRTIs) such as didexoyinosine, dideoxycytidine and azidotymidine. Other combination therapies are envisioned, such as combining DC-SIGN isoforms with protease inhibitors or noriuatoide reverse transcriptase inhibitors (NNRTIs). This is imrtant not only in the creation of more effective therapies, but in reding the chance that drug-resistant viruses will develop.

To inhibit virus replication and thereby limit infecton and T cell Ioss, using the methods and compositions of the present invention, one will treat a patient with a DC-SIGN isoform composition and a traditional antiviral thpeutic. This proess may involve administration of both therapies at the same time, for example, by administration of a single composition or pharmacobgical formulation that includes both agents, or by administering to said patient two distinct compositorns orformulatiorn, at the same time.

Alternatively, the traditional therapy may precede or follow the presnt DSIGN composirion treatment by intervals ranging from minutes to weeks. It is also conceivable that more than one administration of either treatmwentil be desired Various cornatio may be employed, where the DC-SIGN composition is “A” and the traditional therapeutic is “B”:

A/B/A B/A/B B/B/A A/A/B A/B/B B/A/A B/B/B/A
B/B/A/B A/A/B/B A/B/A/B A/B/B/A B/B/A/A B/A/B/A
B/A/A/B A/A/A/B B/A/A/A A/B/A/A A/A/B/A A/B/B/B
B/A/B/B

3. Cancer

In order to increase the effectiveness of DC-SIGN1, DC-SIGN2 or DC-SIGN3 isonrm(s), i may be desrable to combine these compositions and methods of the invention with an agent effective in the treatment of a hfperproftive disease, such as, for exarnple, an anti-cancer agent.

An “anti-cancer” agent is capable of negatively affecting cancer in a sdbject for example, by killing one or more cancer cells, inducing apoptosis in one or more cancer cells, reducing the growth rate of one or more cancer cells, reducing the incidence or number of metastases, reducing a tumor's size, inhibiting a tumor's growth, reducing the blood supply to a tumor or one or more cancer cells, promoting an immune response against one or more cancer cells or a tumor, preventing or inhibiting the progression of a cancer, or increasing the life-span of a subject with a cancer. Anti-cancer agents include, for example, chemotherapy agents (chenotherapy), alkylating agents, radiotherapy agents (radiotherapy), a surgical procedure (surgery), immune therapy agents (immunotherapy), genetic therapy agents (gene therapy), hormonal therapy, other biological agents (biotherapy) and/or altemative therapies that are known to a person of ordinary skill in the art. See for example, the “Physicians Desk Reference”, Goodman and Gilman's “The Pharmacological Basis of Therapeutics”, “Remington's Pharmaceutical Sciences”, and “The Merck Index, Eleventh Edition”).

More generally, such an agent would be provided in a combined amount with an iooform(s) of DC 1, SIG N 2GN and/or DC-SIGN 3 effective to kill or inhibit proliferation of a cancer cell. The proess may irnolve contacting the ce(s) with an agent(s) and DC-SIGN1, DC-SIGN2 and/or DC- SIGN3 isoform(s) at the same time or withn a MW of time wherein separate administration of the DC-SIGN1, DC-SIGN2 and/or DC-SIGN3 isoform(s) and an agent to a celt fbsue or organism produces a desie therapedc benefil This may be achieved by contacting the cell, tissue or organism wih a single composition or pharmaco:i formulation that indudes both a DC-SIGN1, DC-SIGN2 and/or DC-SIGN3 isoform(s) and one or more agents, or by contacting the cel with two or more distinct compositions or formulations, wherein one composition includes a DC-SIGN1, DSIGN2 and/or DC-SIGN 3 isoforrn(s) and the other includes one or more agents.

The terms “contacted” and “exposed,” when applied to a cell, tissue or oraanism, are used herein ID describe the prm by which a therapeutic construct of the present invention and/or another agent, such as for example, a chemote pe or radiotherapeutic agent, are delivered to a target cell, tissue or organism or are placed in direc wda n with the target cell, tissue or organismn.

DC-SIGN1, DC-SIGN2 and/or DC-SIGN3 isoform(s) may precede, be co-current with and/or Low the other agent(s) by intervals ranging from minutes to weeks. In embodiments where DC-SIGN1, DCUSIGN2 and/or DC-SIlG3 isofomi(s), and other agent(s) are applied separately to a cell, tissue or organism, one would generally enure that a signliicant plod of time did not expire between the tme of each delivery, such that DC-SIGN1, DC-SIGN2 and/or DCSSIGN(3) isolb(s) and agents would still be able to exert an advantageously combined effect on the cell, tissue or organism.

Various combination regimens of DC-SIGN isoform(s) and one or more agents may be employec Nondimiting examples of such combinations are shown below, wherein a composition of the present invention is “A” and an agent is “B”:

A/B/A B/A/B B/B/A A/A/B A/B/B B/A/A A/B/B/B
B/A/B/B B/B/B/A B/B/A/B A/A/B/B A/B/A/B
A/B/B/A B/B/A/A B/A/B/A B/A/A/B A/A/A/B
B/A/A/A A/B/A/A A/A/B/A

Administration of the composition to a cell, tissue or organism may follow general protocols for the administration of chemotherapeutics, taking into account the toxicity, if any. It is expected that the treatment cydes would be repated as necessary. In particular embodiments, it is contemplated that various additional agents may be applied in any combination wlih the present invention.

C. Pharmaceutical Formulations and Routes of Administration
1. Pharmaceutical Compositions

Where clinical applications are contemplated, it will be necessary to prepare pharital composions of the cornpositins in a form appropriate for the intended application. Generally, this will entail prepari omposingons that are essentially free of pyrogens, as well as other impurities that could be harmful to humans or animals.

Solutions of the compositions as free base or pharmacologically acceptable sals may be prepared in water suiably mixed with a surfactant, such as hydroxypropylcellulose. Dispersions may also be prepared in glycerol, iquid polyethylene gtyoos, and mixtures thereof and in oils. Under ordinary conditions of storage and use, these preparations contain a presevative to prevent the growth of microorganisms. The pharmaceutical forms suitable for injectable use include stenle aqueous solutions or dispersions and sterile powders for the extemporaneous preparation of sterile injectable solutions or dispersions (U.S. Pat. No. 5,466,468). In all cases the form must be sterile and must be fluid to the extent that easy syringability exist. t must be stable under the conditions of manufacture and storage and must be preserved against the contaminating action of microorganisms, such as bacteria and futgi. The canier can be a solvent or dispersion medium containing, for example, water, ethanol, polyol (e.g., glycerol, propylene glycol, and lquid polyethytene glycol, and the like), suitable mixtures thereof, and/or vegetable oils. Proper fluidity may be maintained, for example, by the use of a coating, such as lecithin, by the maintenance of the required particle size in the case of dispersion and by the use of su iactants. The prevention of the action of microorganisms can be brought about by various antibacterial and antig agents, for example, parabens, chlorobutanol, phenol, sorbic acid, thimerosal, and the like. In many cases, it wil be preferable to i isotonic agents, for example, sugars or sodium chloride. Prolonged absorption of the injectable compositions can be brought about by the use in the compositions of agents delaying absorption, for example, alminium monostearate and gelatin.

Sterile injectable solutions are prepared by incorporating the active compounds in the r amount in the appropriate solvent with various of the other ingredients enumerated above, as required, followed by filtered sterifiba GenraeDy, dispersions are prepared by incorporating the various sterilized active ingredients into a sterile vehicle which contains the basic dispersion medium and the required other ingredients from those enumerated above. In the case of sterile powders for the preparation of stenle injectable solutions, the preferred methods of preparation are vacuum-drying and freeze-drying techniques wic yield a powder of the active ingredient plus any additional desired ingredient from a previously sterile-filtered solution theof.

The compositions disclosed herein may be formulated in a neutral or salt forml P l s a c ea, ce the acid addition salts and which are formed with inorganic acids such as, for example, hydrobi or pphoric acids, or such organic acids as acetic, oxalic, tartaric, mandelic, and the like. Salts formed with the ftee carbo4x groups can also be derived from inor bases such as, for example, sodium, potassium, ammonium, calcium, or ferric hydroxides, and such organic bases as isopropybmine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like.

As used herein, “carrier” includes any and all solvents, dispersion media, vehicles, coailgs, diluents, antibacterial and antitfungal agents, isotonic and absorption delaying agents, buffers, carrier solutions, suspensions, colioka and the like. The use of such media and agents for pharmaeutical active substances is well known in the art Except insofar as any conventional media or agent is incompatble with the active ingredient, its use in the therapeutic compositions is contemplated. Supplementary actve ingredients can also be incorporated into the compositions.

The phrase “pharmaceutically-acceptable” or “pharmacologically-acceptable” refers to modutar entis and composirions that do not produce an allergic or similar untoward reaction when adminislered to a human. The problem of an aqueous composition that contains a protein as an active ingredient is well understood in the art. Typically, such composits are prepared as injectables, either as liquid solutions or suspensions; solid forms suitable for solution in, or suspension in, UqLid prior to injection can also be prepared.

The terms “contacted” and “exposed,” when applied to a cell, are used herein to descnbe the Isoess by which a therapeutic viral vector or a composition is delivered to a target cell.
2. Routes of Administration

The routes of adminitration will vary, naturally, with the location and nature of the disease, and include, eg., intrenous, intraterial, intradermal, transdermal, intramuscular, intranasal, subcutaneous, percutaneous, intratracheal, intrapertoneal, intratumoral, perfusion and lavage. The cells will also sometimes be isolated from the organisms, exposed to the vector ex uw, and rmplarted afterwards.

Injection of the compositions of the invention may be delivered by syringe or any other method used for injection of a solution, as long as the expression construct can pass through the particular gauge of needle required for injection. A novel needle less injection system has recently been described (U.S. Pat. No. 5,4623) having a nozzle defining an ampule chamber for holding the solution and an energy device for pushing the soluton out of the nozzle to the site of delivery. A syringe system has also been descnbed for use in gene therapy that permits multiple injections of predetermined quantities of a solution precisely at any depth (U.S. Patent 5,846,225).

For parenteral administration in an aqueous solution, for example, the solution should be suitably buffered if necessary and the liquid diluent first rendered isotonic with sufficient saline or glucose. These particular aqueous solutions are especially suitable for intravenous, intraarterial, intramuscular, subcutaneous, intratumoral and intraperitoneal administaton. In this oonnection, sterile aqueous media that can be employed will be known to those of skill in the art in light of the presentidisc sureF For cample, one dosage may be dissolved in 1 ml of isotonic NaCl solution and either added to 1000 ml of hypodemocl fluid or ir-ed at the proposed site of infusion, (see for example, “Remington's Pharmaceutical Sciences”15th Editon, pages 1035-1038 and 1570-1580). Some variation in dosage will necessarily occur depending on the condition of the subject being treated. The person responsibl for administration wiU, in any event, determine the appropriate dose for the individual subject. Moreover, for human administration, preparations should meet sterility, pyrogenicity, general safety and purity standards as required by FDA Offlice of Biologics standards.

Continuous administration also may be applied where appropriate. Delivery tb syringe or tahenzaton is preferred. Such continuous perfusion may take place for a period from about 1-2 hours, to about 2X hours, to about 6-12 hours, to about 12-24 hours, to about 1-2 days, to about 1-2 wk or longer following the initiation of tealrnent Generaly, the dose of the therapeutic compost via continuous perfusion will be equivalent to that given by a single or multiple injections, adjusted over a period of time during which the perfusion occurs.

In addition to the compounds formulated for parenteral administration, oral formulations are pried. These compositions take the form of solutions, suspensions, tablets, pills, capsules, sustained release formutations or powders and include such typical excipients as, for example, phamaceutical grades of mannitol, lactose, starch, magnesium stearate, sodium saccharine, cellulose, magnesium carbonate and the like. Similar compositions are provided or nasal, buocalt rectal, vaginal or administration.

Treatment regimens may vary as well, and often depend on type of diseae and ocation of diseased tisse, and fccrs such as the health and the age of the patient. The clinican will be best suited to make sch decisions based on the kowneficacyand toxity (if any) of the therapeutic formulations based on viral vectors of the present inventon.

The treatments may include various “unit doses.” A unit dose is defined as containing a predeterrined-quantty of the therapeutic composition comprising a viral vector or other compositions of the present invention The quantity to be administered, and the particular route and formulation, are within the skill of those in the clnical arts A unit dose need not be administered as a single injection but may comprise continuous infusion over a set period of time. Unit dose of the present invention may conveniently be described in terms of transducing units (T.U.) of vector, as defined by tittering the vector on a cell line such as HeLa or 293. Unit doses range from 10³, 10⁴, 10⁵, 10⁶, 10⁷, 10⁸, 10⁹, 10¹⁰, 10¹¹, 10¹², 10¹³ T.U. and higher.

D. Antibodies
1. Antibody Generation

It will be understood that polyclonal or monoclonal antibodies specific for DC-SIGN isoforms and reated proteins wiU have utilities in several applications. These include the reduction or prevention of viral infection in a subect production of diagnostic kits for use in detecting and diagnosing conditions and diseses involving the activities or presnce of DC-SIGN isoforms. An additional use is to link such antibodies to therapeutic agents, such as chemotherapeutic agents, and to administer the antibodies to individuals with disease, thereby selectively targeting the selected cells for destruction.

Thus the invention further provides antibodies specific for the proteins, p(*etides or peptides, such as iCSIGN, encoded by the nucleic acid segment disclosed herein and their equivalents. Means for preparing and charactenzing antibodies are well known in the art (See, e.g., Harlow and Lane, 1988, U.S. Pat. No. 5,565,332 which dies the production of chimeric antibodies using a combinatorial approach; U.S. Pat. No. 4,816,567 which describes recombinant immunoglobin preparations and U.S. Pat. No. 4,867,973 which describes antibody-therapeutic agent conjugates; U.S. Pat. No. 5,565,332 debes methos for the production of antibodies, or antibody fragments, which have the same binding specifically as a parent anbbody but which have increesed human characteristics; Suresh et al., (1986) describes methods and production of - ic antibodies.
i. Polyclonal Antibodies

Briefly, a polyclonal antibody is prepared by immuning an animal with an immunogenic composion in aoordance with the present invention and collecting antisera from that immunized animal. A wide range of anirl speaes can be used for the producton of antisera. Typically the animal used for production of andi-antisera is a rabbit, a rnouse, a rat a hamster, a guinea pig or a goat Because of the relatively large blood volume of rabbits, a rabbit is a preferred choice for pmckdon of pa cnal antibodies.
ii. Monoclonal Antibody Production

MAbs may be readily prepared through use of well-known techniques (see e.g, Kozbor, 1984; Brodeur, et al, 1987; U.S. Pat. No. 4,196,26. Typically, this technique involves immunizing a suitabl animal with a selected immunogen composition, e.g., a purified or partialy purified DC-SIGN protein, polypeptde or peptide. The immunizing composition is administered in a manner efeclive to stimulate antibody producing cells.
iii. Humanized Antibodies

Humanized monoclonal antibodies are antbodies of animal origin that have been modifled using genetic engineering techniques to replace constant region and/or variable region framework sequences with human sequences, while retaining the original antigen specificity. Such antibodies are commonly derived from rodent antibodies with i again human antens and are useful for in vivo therapeutic applications. This strategy reduces the host response to the frign antboly and allows selecion of the human effector functions.

The techniques for producing humanized immunoglobulins are well known to those of ski in the art For example U.S. Pat. No. 5,693,762 discloses methods for producing, and compositions of, humanized immunoglobulins having one or more complementary determining regions (CDR's). When combined into an intact antibody, the humanized immunoglobulins are substantialy non-immunogenic in humans and retain substantially the same affinity as the donor immunoglobulin to the antigen, such as a protein or other compound containing an epitope.
2. Antibody Conjugates

Antibody conjugates in which a DC-SIGN antibody is linked to a detectable be or a cytotoxic ageft form frer aspecs of the invention. Diagnostic antibody conjugates may be used both in vitro diagnostics, as in a variety of nwsys, and in v diagnostics, such as in imaging technobgy.

Certain antibody conjugates include those intended primarily for use in vitro, where the antbody is linked to a secondary binding ligand or to an enzyme (an enzyme tag) that will generate a colored product upon contact wth a chromogenic substrate. Examples of suitable enzymes include urease, alkaline phosphatase, (horseadish) hydrogen peroxd and glucose o)ddase. Preferred secondary binding ligands are biotin and avidin or streptavidin compounds. The use of such labs is well known to those of skill in the art in light and is described, for example, in U.S. Pat. Nos. 3,817,837; 3,850,752; 3,939,350; 3,996,345, 4,277,437; 4,275,149 and 4,366,241. Many appropriate imaging agents are also known in the art, as are methods for their aftactiii to antibodies (see, e.g., U.S. Pat. Nos. 5,021,236 and 4,472509).
3. Immunotoxins

The invention further provides immunotoxins in which an antibody that binds to a DCGSIGN Aptide or nucleic acid is linked to a cylotoxic agent. Immunotoxin technology is fairly well-advanced and and known to those of skill in fee art lmrnunotoxs are agents in which the antibody component is linked to another agent, particularly a cytotoxic or othenwse anktar agent having the ability to kill or suppress the growth viruses or cell division of cells.

Toxins are thus pharmacologic agents that can be conjugated to an antibody and delivered in an active form to a cell or other target, wherein they will exert a significant deleterious effect. The preparation of immunotoxins is, in general, well known in the art (see, e.g., U.S. Pat. No. 4,340,535).

E. Immunological Detection
1. Immunoassays

The antibodies of the invention, as exemplified by anti-DC-SIGN antibodies, are useful in varous diagnostic and prognostic applications connected with the detection and analysis of diease.

The steps of various useful immunodetection methods have been described in the scientific raabne such as, e.g, Nakamura et al. (1987). Immunoassays, in their most simple and direct sense, are binding assays Certain prefer w imrnuoassas are the various types of enzyme linked immunosorbent assays (ELISAs), radioimmunoassays (RIA) and immunobead capture assay. immunohistochemical detection using tissue sections also is particularly useful. However, it wil be readily acted that detecon is not limited to such techniques, and Western botting, dot blotting, FACS analyses, and the WBe also may be wW in connection with the present invention.

In general, immunobinding methods include obtaining a sample suspected of containing a pin peptide or anfbuii, and contacting the sample with an antibody or protein or peptide in accordance wth the preent invention, as th case may be, under conditions effective to allow the formation of immunocomplexes.

The immunobinding methods of this invention include methods for detecting or quantifying the amount of a reactive component in a sample, which methods require the detection or quantitation of any immune complexes *xmed during the binding process. Here, one would obtain a sample suspected of containing a DC-SIGN, peptide or a corresponding antibody, and contact the sample with an antibody or encoded protein or peptide, as the case may be, and then detect or quantl the amount of immune complexes formed under the specific conditions.

In general, the detection of immunocomplex formation is well known in the art and may be achieved through the application of numerous approaches. These methods are generally based upon the detection of a lbel or marker, such as any racdmtive, fluorescent, biological or enzymatic tags or labels of standard use in the art U.S. Patents coneming the use of such labels include U.S. Pat. Nos. 3,817,837; 3,850,752; 3,939,350; 3,996,345; 4,277,437; 4,275,149 and 4,366,241. Of course, one may find additional advantages through the use of a secondary binding ligand such as a second antibody or a bi avidin lgand binding arngement, as is no in the art.
2. Immunohistochemistry

The antibodies of the present invention, such as an anti-DC-SIGN antibodies, also may be used in conjunction with both fresh-frozen and formalin-fixed, parafin-embedded tissue blocks prepared frm study by inmunohistochemistiy (IHC) (Brown et al., 1990;Abbondanzo et al., 1990; Allred et al., 1990).
3. FACS Analyses

Fluorescent activated cell sorting, flow cytometry or flow microfluorometry provides the means of scanning indvidual cells for the presence of DC-SIGN isoforms. The method employs instrumentation that is capable of acffva, and detecting the excitation emissions of labeled cells in a liquid medium.

F. Nucleic Acids

One embodiment of the present invention is to transfer nucleic acids encoding DC-SIGN 1, 2 or 3 isoforms to provide therapy for viral infections, non-viral infections, bacterial infections, cancer and for hematopoietic and blmphn hematopoietc discoders In one embodiment the nucleic acids encode a full-length, substantially full-length, or funcanal equivalent form of a DC-SIGN isoform. In other embodiments, the nuclic acids encode non full-length DC-SIGN isoforms.

A nucleic acid may be made by any technique known to one of ordinary skill in the art Nonimlitg examples of synthetic nucleic acid, particularly a synthetic oligonucleotide, include a nucleic acid made by in v chemical synthesis using phosphotiester, phosphite or phosphoramdite chemistry and solid phase techniques such as described in EP 266,032 or via deoxynuclecside H-phosphonate intermediates as described by Froehler et al., 1986, and U.S. Pat. No. 5,705,629. A non-limiting example of enzymatically produced nucleic acid include one produced by enzymes in amplification reactons such as PCRT (see for exarnple, U.S. Pat. No. 4,683.202 and U.S. Pat. No. 4,682,195), or the synthesis of oligonucleotides described in U.S. Pat. No. 5,645,897. A non-limiting example of a biologicaly produced nucleic acid includes recombinant nucleic acid production in living cells (see for example, Sambrook et al. 2001).

A nucleic acid may be purified on polyacrylamide gels, cesium chloride centrifugation gradients, or by any other means Iwwi to one of ordinary skill in the art (Sambrook et al. 2001).

The term “nucleic acid” will generally refer to at least one molecle or strand of DNA, RNA or a dema or mimic thereof, comprising at least one nucleobase, such as, for example, a naturally occurring purine or pyrimidine base found in DNA (e.g., adenine “A,” guanine “G,” thymine “T,” and cytosine “C”) or RNA (e.g. A, G, uracil “U,” and C). The term “nucleic acid” encompasses the terms “oligonucleotide” and “polynucleotide.” The term “oligonucliotide” refers to at least one molecule of between about 3 and about 100 nuclobases in length. The term “polynucleotide” refers to at least one rclecule of greaterthan about 100 nucleobases in length. These definitions generally refer to at least one single-stranded molecule, but in specific embodiments will also encompass at least one additional strand that is partially, substantially or fully complementary to the at least one singl-stranded molecule. Thus, a nucleic acid may encompass at least one double-stranded molecule or at least one triple-straie molecule that comprises one or more complementary strand(s) or “complement(s)” of a particular sequence comprising a strand of the molecule.

In certain embodiments, a “gene” refers to a nucleic acid that is transcribed. As used herein, a “gene segment” is a nucleic acid segment of a gene. In certain aspects, the gene includes regulatory sequences invoed in trcription, or message production or composition. In particular embodiments, the gene comprises transcribed sequences that eocode for a protein, polypeptide or peptide. In other particular aspects, the gene comprises a nucleic acid, and/or encodes a potypeptide or pepi e ucing sequences of DC-SIGN isoforms. In keeping with the technology described herein, an “isolated gene” may comrpris transcribed nucleic acid(s), regulatory sequences, coding sequences, or the like, isolated substantially away from other such sequences, such as other naturally occluring genes, regulatory sequences, polypeptide or peptide encoding sequences, etc. In this respect, the term “gene” is used for simplicity to refer to a nucleic acid comprising a nucleotide sequence that is transcribed, and the cmplement thereof. In particular aspects, the transcribed nucleotide sequence comprises at least one functional protein, polypeptide and/or peptide encoding unil As will be understood by those in the art, this functional term “gene” includes both genomic sequences, RNA or CDNA sequences, or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, tusion proteins, mutants and/or such like. Thus, a “truncated gene” refers to a nucleic acid sequencethat is sing a stIh of contiguous nucleic acid residues.

Various nucleic acid segments may be designed based on a particular nucleic acid sequence, and may be of any length. By assigning numeric values to a sequence, for example the first residue is 1, the second residue is 2, etc., an algorithm defining all nuceic acid segments can be created:
n to n+y
when n is an integer from 1 to the last number of the sequence and y is the length of thenucleic acid segment minus one, where n+y does not exceed the last number of the seqeence. Thus, for a 10-mer, thenucleicacid segmentsoorrespondto bases 1 to 10, 2 to 11, 3 to 12 . . . and/or so on. For a 15-mer, the nucleic acid segments correspond to bases I to 15, 2to 16, 3to 17 . . . and/or so on. For a 20-mer, the nucleic segments correspond to bases 1 to 20, 2 to 21, 3 to 22 . . . and/or so on.

The nucic acid(s) of the present invention, regardless of the length of the sequence itself, may be combined with other nucleic acid sequences, including but not limited to, promoters, enhancers, pyadenylation signals, restriction enzym sites, multiple cloning sites, coding segments, and the like, to create one or more nucleic acid construct(s). The overall length may vary considerably between nucleic acid constructs. Thus, a nucleic acid segment of almost any length may be employed, with the total length preferably being limited by the ease of preparation or use in the intended recombinant nucleic acid protocol.
1. Vectors

The term “vector” is used to refer to a carrier nucleic acid molecule into which a nucleic acid sequence can be inserted for introduction into a cell where it can be replicated. Vectors of the present invention are virs based as described above and in other parts of the specification. The nucleic acid molecules carried by the vectors of the inverioon e=Wode therapeutic genes and will be used for carrying out gene-therapies. One of skill in the art would be well equipped to construct such a therapeuic vector through standard recombinant techniques (see, for example, Maniatis et al., 1988 and Ausubel et al., 1994).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the protion of antisense molecules or riboyrnes. Expression vectors can contain a variety of “control sequences,” which refer to nucleic acid sequences nssary for the tanscription and possibly translation of an operably linked coding sequence in a particular host cell. In addition to control sequences that govern transcription and translation, vectors and expression vectors may contain nucleic acid sequences that serve other functions as well and are described below.
2. Promoters and Enhancers

A “promoter” is a control sequence that is a region of a nucleic acid sequence at which initiation and rate of transcription are controlled. It may contain genetic elements at which regulatory proteins and molecdes may bind, such as RNA polymerase and other transcription factors, to initiate the specific tanscription a nucleic acid sequence. The phrases “operatively positioned,” “operatively linked,” “under control,” and “under transcriptional control” mean that a promoter is in a correct functional boation and/or orientation in relation to a nucleic acid sequence to control transcriptional initiation and/or exession of that sequence.

A promoter generally comprises a sequence that functions to position the start site for RNA synthesis. The best known example of this is the TATA box, but in some promoters lackng a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation Typlcail, thee are located in the region 30-110 bp upstream of the start site, althou a number of poioters have been shown to contain functional elements downstream of the start site as well. To bring a coding sequence “under the control of” a promoter, one positions the 5′ end of the transcription initiation site of the transcriptional reading frame “downstream” of (i.e. 3′ of) the chosen piriorter. The “upstream” promoter stimulates transcription of the DNA and promotes expression of the encoded RNA.

The spacing between promoter elements frequently is flexible, so that promter function is preserved when elements are inverted or moved relative to one another. In the tk promoter, the spacing between promoter elements can be increased to 50 bp apart before activity begins to decline. Depending on the promoter, it appears that idivvlual elements can ftion either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located elier downstear or upsteam of that sequence. Alternatively, certain advantages will be gained by positioning the coding nucleic acid segment under the control of a recombinant or heterologous promoter, which refers to a promoter that is not normaly associated with a nucleic acid sequence in its natural environment. A recombinant or heterologous enhancer refers also to an enhancer not normally associated with a nucleic acid sequence in its natural environment. Such promoters or enhancers mayu include promoters or enhancers of other genes, and promoters or enhancers isolated from any other virus, or prokaryotic or eukaryotic cell, and promoters or enhancers not “naturallyo occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection witht he compositions disclosed herein (see U.S. Pat. Nos. 4,683,202 and 5,928,906). Furthermore, it is contemplated the control sequences that direct transcription and/or expression of sequences within non-nuclear organelles such as mitochondria, chloroplats, and the like, can be employed as well. Control sequences comprising promoters, enhancers and other locus or transcription controlling/modulating elemens are also referred to as “transcriptional cassettes”.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al., 2001). The promoters employed may be constitutive, tissue-specific, inducible, and/or useful under the appropriate conditions to direct high level expression of the introduced DNA segment, such as is advantageous for gene therapy or for applications such as the large-scale production of recombinant proteins an/or peptides. The promoter may be heterologous or endogenous.

Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Tables 1 lists non-limited examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin Heavy Chain | Banerji et al., 1983; Gilles |
| | et al., 1983; Grosschedl et al., |
| | 1985; Atchinson et al., 1986, |
| | 1987; Imler et al., 1987; |
| | Weinberger et al., 1984; |
| | Kiledjian et al., 1988; Porton |
| | et al.; 1990 |
| Immunoglobulin Light Chain | Queen et al., 1983; Picard et |
| | al., 1984 |
| T-Cell Receptor | Luria et al., 1987; Winoto et |
| | al., 1989; Redondo et al.; 1990 |
| HLA DQ a and/or DQ β | Sullivan et al., 1987 |
| β-Interferon | Goodbourn et al., 1986; Fujita |
| | et al., 1987; Goodbourn et al., |
| | 1988 |
| Interteukin-2 | Greene et al., 1989 |
| Interteukin-2 Receptor | Greene et al., 1989; Lin et |
| | al., 1990 |
| MHC Class II 5 | Koch et al., 1989 |
| MHC Class II HLA-Dra | Sherman et al., 1989 |
| β-Actin | Kawamoto et al., 1988; Ng et |
| | al.; 1989 |
| Muscle Creatine Kinase (MCK) | Jaynes et al., 1988; Horlick |
| | et al., 1989; Johnson et al., |
| | 1989 |
| Prealbumin (Transthyretin) | Costa et al., 1988 |
| Elastase I | Omitz et al., 1987 |
| Metallothionein (MTII) | Karin et al., 1987; Culotta |
| | et al., 1989 |
| Collegenase | Pinkert et al., 1987; Angel |
| | et al., 1987 |
| Albumin | Pinkert et al., 1987; Tronche |
| | et al., 1989, 1990 |
| α-Fetoprotein | Godbout et al., 1988; Campere |
| | et al., 1989 |
| γ-Globin | Bodine et al., 1987; Perez- |
| | Stable et al., 1990 |
| β-Globin | Trudel et al., 1987 |
| c-fos | Cohen et al., 1987 |
| c-HA-ras | Triesman, 1986; Deschamps et |
| | al., 1985 |
| Insulin | Edlund et al.,1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and | |
| GRP78) | |
| Rat Growth Hormone | Larsen et al., 1986 |
| Human Serum Amyloid A(SAA) | Edbrooke et al., 1989 |
| Troponin I (TN I) | Yutzey et al., 1989 |
| Platelet-Derived Growth | Pech et al., 1989 |
| Factor (PDGF) | |
| Duchenne Muscular Dystrophy | Klamut et al., 1990 |
| SV40 | Banerji et al., 1981; Moreau |
| | et al., 1981; Sleigh et al., |
| | 1985; Firak et al, 1986; Herr |
| | et al., 1986; Imbra et al., |
| | 1986; Kadesch et al., 1986; |
| | Wang et al, 1986; Ondek et al., |
| | 1987; Kuhl et al., 1987; |
| | Schaffner et al., 1988 |
| Polyoma | Swartzendruber et al., 1975; |
| | Vasseur et al., 1980; Katinka |
| | et al., 1980, 1981; Tyndell et |
| | al., 1981; Dandolo et al., 1983; |
| | de Villers et al., 1984; Hen et |
| | al., 1986; Satake et al., 1988; |
| | Campbell and/or Villarreal, 1988 |
| Retroviruses | Kriegler et al., 1982, 1983; |
| | Levinson et al., 1982; Kriegler |
| | et al., 1983, 1984a, b, 1988; |
| | Bosze et al., 1986; Miksicek et |
| | al., 1386; Celander et al., 1987; |
| | Thiesen et al., 1988; Celander |
| | et al., 1988; Chol et al., 1988; |
| | Reisman et al., 1989 |
| Papilloma Virus | Campo et al., 1983; Lusky et al., |
| | 1983; Spandidos and/or Wilkie, 1983; |
| | Spalholz et al., 1985; Lusky et |
| | al., 1986; Cripe et al., 1987; |
| | Gloss et al., 1987; Hirochika et |
| | al., 1987; Stephens et al., 1987 |
| Hepatitis B Virus | Bulla et al., 1986; Jameel et al., |
| | 1986; Shaul et al., 1987; Spandau |
| | et al., 1988; Vannice et al., 1988 |
| Human Immunodeficiency | Muesing et al., 1987; Hauber et |
| Virus | al., 1988; Jakobovits et al., 1988; |
| | Feng et al, 1988; Takebe et al., |
| | 1988; Rosen et al., 1988; Berkhout |
| | et al., 1989; Laspia et al., 1989; |
| | Sharp et al., 1989; Braddock et |
| | al., 1989 |
| CD11b | Hickstein et al., 1992 |
| Gibbon Ape Leukemia Vims | Holbrook et al., 1987; Quinn |
| | et al., 1989 |

TABLE 2
Inducible Elements

| Element | Inducer | References |
|---|---|---|
| MT II | Phorbol Ester | Palmiter et al., 1982; |
| | (TFA) | Haslinger et al., 1985; |
| | Heavy metals | Searle et al., 1985; Stuart |
| | | et al., 1985; Imagawa et |
| | | al., 1987, Karin et al., |
| | | 1987; Angel et al., 1987b; |
| | | McNeall et al., 1989 |
| MMTV (mouse | Glucocorticoids | Huang et al., 1981; Lee et |
| mammary | | al., 1981; Majors et al., |
| tumor virus) | | 1983; Chandler et |
| | | al., 1983; Lee et al., 1984; |
| | | Ponta et al, 1985; Sakai et |
| | | al., 1988 |
| β-Interferon | Poly(rl)x | Tavernier et al., 1983 |
| | Poly(rc) | |
| Adenovirus | EIA | Imperiale et al., 1984 |
| 5 E2 | | |
| Collagenase | Phorbol Ester | Angel et al., 1987a |
| | (TPA) | |
| Stromelysin | Phorbol Ester | Angel et al., 1987b |
| | (TPA) | |
| SV40 | Phorbol Ester | Angel et al., 1987b |
| | (TPA) | |
| Murine MX | Interferon, | Hug et al., 1988 |
| Gene | Newcastle | |
| | Disease Virus | |
| GRP78 Gene | A23187 | Resendez et al., 1988 |
| α-2-Macro- | IL-6 | Kunz et al., 1989 |
| globulin | | |
| Vimentin | Serum | Rittling et al., 1989 |
| MHC Class I | Interferon | Blanar et al., 1989 |
| Gene H-2κb | | |
| HSP70 | EIA, SV40 Large | Taylor et al., 1989, |
| | T Antigen | 1990a, 1990b |
| Proliferin | Phorbol Ester- | Mordacq et al., 1989 |
| | TPA | |
| Tumor Necrosis | PMA | Hensel et al., 1989 |
| Factor | | |
| Thyroid Stimu- | Thyroid Hormone | Chatterjee et al., 1989 |
| lating Hormone | | |
| α Gene | | |

The identity of tissue-specific promoters or elements, as well as assays to characterize their activity, is well known to those of skill in the art. Non-limiting examples of such regions include the human LIMK2 gene (Nomoto et al., 1999), the somatostatin receptor 2 gene (Kraus et al., 1998), murine epididymal retinoic acid-binding ene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

The viral vectors of the present invention are designed, primarily, to transform cells with a therapeutic gene under the control of regulated eukaryotic promoters. Although the gp91-phox promoter is preferred, other promoter and regulatory signal elements as d described in the Tables 1 and 2 above may also be used. Additionally any promoter/enhancer combination (as pr the Eukaryotic Promoter Data Base EPDB) could also be used to drive expression of structural genes encoding the terapeutic gene of interest that is used in context with the viral vectors of the present invention. Alternatively, a tissue-specific promoter or cancer gene therapy or the targeting of tumors may be employed with the viral vectors of the present invention for treatment of cancers, especially hematological cancers.

Typically, promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. Activation or repression of the promoter and enhancer elements may be had through contacting those elements with the appropriate transcriptional activators or repressors, such as those disclosed in Luo and Skalnik (1996a; 1996b). With respect to the gp91-phox promoter, the activity of Interferon-gamma in modulating the transcription and expression of the expression cassette is an example of how such promoter or enhancer elements and the factors that interact with them may be employed in the practice of the present invention.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more tanscriptional proteins. See, for example, the model for the regulation of the gp91-phox promoter presented in FIG. 1B. Exemplary enhancers contemplated in the present invention are the DNAase HyperSensitive elements and their homologs described by Lien et al., (1997) “Regulation of the myeloid-cell-expressed human gp91-phox gene as studied by transfer of yeast artificial chromosome clones into embryonic stem cells: suppression of a variegated cellular pattern of expression requires a full complement of distant cis elements”. Under the influence of these enhancer elements, gene expression may be higher (due to enhancer activity HS) and less variegated (due to silencer actvity of HS).

Analogs of the HS elements of gp91-phox are active in other promoter-enhancer systems. See, for example, May et al. (2000). Therapeutic haemoglobin synthesis in beta-thalassaemic mice expressing virus-encoded human beta-globin, where analogous beta-globin HS elements were included into a viral vector upstream of beta-globin promoter to drive expression of beta-globin cDNA.

Promoters and enhancers have the same general function of activating transcription in the cell. They are often overlapping and contiguous, often seeming to have a very similar modular organization. Taken together, these considerations suggest that enhancers and promoters are homologous entities and that the transcriptional activator proteins bound to these sequences may interact with the cellular transcriptional machinery in fundamentally the same way. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Aside from this operational distinction, enhancers and promoters are very similar entities. Constructs of elements that control transcription and expression may therefore be c comprised of various elements arranged so as to provide means of control of enhanced utility and operation.

A signal that may prove useful is a polyadenylation signal (hGH, BGH, SV40). The use of internal ribosome binding sites (IRES) elements are used to create multigene, or polycistronic, messages. IRES elements are able to bypass the ribosome scanning model of 5 [symbol] methylated cap-dependent translation and egin translation at internal sites (Pelletier and Sonenberg, 1988). IRES elements from two members of the picomavirus family (polio and encephalomyocarditis) have been described (Pelletier and Sonenbreg, 1988), as well as an IRES from a mammalian message (Macejak and Sarrow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

In any event, it will be understood that promoters are DNA elements that when positioned functionally upstream of a gene leads to the expression of that gene. Most transgenes that will be transformed using the viral vectors of the present invention are functionally positioned downstream of a promoter element.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.
3. Multiple Cloning Sites

Vectors of the present invention can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997) Restriction enzyme digestion refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of formong phosphodiester bonds between two nucleic acid fragments, which may or may not be contiguous with each other. Techniques involving restriction enzymes and ligation reactions are well known to those of skill in the art of recombinant technology.
4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997).
5. Termination Signals

The vectors or constructs of the present invetion will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to move stable and are translated more efficiently. Thus, in other embodiments involving eukaryotes, it is preferred that that terminator comprises a signal for the cleavage of the RNA, and it is more preferred tha the trminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.
6. Polyadenylation Signals

In eukaryotic gene expression, one will typically include a polyadenylation signal to effect proper polyadenylation of the transcript. The nature of the polyadenylation signal is not believed to be crucial to the successful practice of the invention, and any such sequence may be employed. Some examples include the SV40 polyadenylation signal or the bovine homone polyadenylation signal, convenient and known to function well in varius target cells. Polyadenylation may increase the statility of the transcript or may faclitate cytoplasmic transport.
7. Origins of Replication

In order to propagate a vector of the invention in a host cell, it may contain one or more origins of replicaton sites (often termed “on”), which is a specific nucleic acid sequence at which replication is initiated. Alternatively, an autonomously replicating sequence (ARS) can be employed if the host cell is yeast.
8. Selectable and Screenable Markers

In certain embodiments of the invention, cells transduced with the vectors of the present invention may be identfied in vitro or in vivo by including a marker in the exon vector. Such markers would confer an identifiable change to the transduced cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genetic constructs that confer resistance to neomycin, puromycin, hygromycin, DHFR, GPT, zeocin and histidinol are useful selectable markers. In addition to markers conferring a phenotype that allows for the discrimination of ttransformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenical acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectble and screenable markers are well known to one of skill in the art.
9. Assays of Gene Expression

Assays may be employed within the scope of the instant invention for determination of the relative efficiency of gene expression. For example, assays may be used to determine the efficacy of deletion mutants of specific promoter regions in directing expression of operably linked genes. Similarly, one could produce random or site-specific mutants of promotre regions and assay the efficacy of the mutants in the expression of an operably linked gene. Alternatively, assays could be used to determine the function of a promoter region in enhancing gene expression when used in conjunction with various different regualtory elements, enhancers, and exogenous genes.

Gene expression may be determined by measuring the production of RNA, protein or both. The gene product (RNA or protein) may be isolated and/or detected by methods well known in the art. Following detection, one may compare the results seen in a given cell line or individual with a statistically significant reference group of non-transformed control cells. Alternatively, one may compare production of RNA or protein products in cell lines transformed with the same gene operably linked to various mutants of a promoter sequence. In this way, it is possible to identify regulatory regions within a novel promoter sequence by their effect on the expression of an operably linked gene.

G. Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous nucleic acid encoded by the vectors of this invention. A host cell can, and has been, used as a recipient for vectors. A host cell may be “transfected” of “transformed,” which refers to a cprocess by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector of the invention bearing a therapeutic gene construct, has been introduced. Therefore, recombinant cells are distinguishable from naturally occurring cells which do not contain a recombinantly introduced nucleic acid.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). Some examples of host cells used in this invention include but are not limited to virus packaging cells, virus producer cells, 193T cells, human hematopoietic progenitor cells, human hematopoietic stem cells, CD34+cells CD4+cells, and the like.
1. Tissues and Cells

A tissue may comprise a host cell or cells to be transformed or contacted with a nucleic acid delivery composition and/or an additional agent. The tissue may be part or separated from an organism. In certain embodiments, a tissue and its constituent cells may comprise, but is not limited to, blood (e.g., hematopoietic cells (such as human hematopoietic progenitor cells, human hematopoietic stem cells, CD34+cells CD4+cells), lymphocytes and other blood lineage cells), bone marrow, brain, stem cells, blood vessel, liver, lung, bone, breast, cartilage, cervix, colon, cornea, embryonic, endometrium, endothelial, epithelial, esophagus, facia, fibroblast, folicular, ganglion cells, glial cells, goblet cells, kidney, lymph node, muscle, neuron, ovaries, pancreas, peripheral blood, prostate, skin, skin, small intestine, spleen, stomach, testes.
2. Organisms

In certain embodiments, the host cell or tissue may be comprised in at least one organism. In certain embodiments, the organism may be, human, primate or murine. In other embodiments the organism may be any eukaryote or even a prokayote (e.g., a eubacteria, an archaea), as would be understood by one of ordinary skill in the art (see, for example, webpage http://phylogeny.arizona.edu/tree/phylogny/html). Some vectors of the invention may employ control sequences that allow them to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of the vectors of the invention, as well as production of the nucleic acids encoded by the vectors and their cognate polypeptides, proteins, or peptides some of which are therapeutic genes or proteins which will be used for gene therapies.

H. Proteinaceous Compositions

In certain embodiments, the present invention concerns novel compositions comprising at least one proteinaceous molecule such as a DC-SIGN 1, 2 or 3 polypeptide isoform. As used herein, a “proteinaceous molecule,” “proteinaceous composition,” “proteinaceous compound,” “proteinaceous chain” or “proteinaceous material” generally refres, but is not limited to, a protein of greater than about 200 amino acids or the full lenth endogenous sequence translated from a gene; a polypeptide of greather than about 100 amino acids; and/or a peptide of from about 3 to about 100 amino acids. All the “proteinaceous” terms described above may be used interchangeably herein.

In certain emodiments the size of the at least one proteinaceous molecule may comprise, but is not limited to, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 25, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59 about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about90, about 91, about 92, about 93, about 94, about 95, about 96, about 97, about 98, about 99, about 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, abot 45, about 475, about 500, about 525, abt 550, about 575, about 600, about 625, about 650, about 675, about 700, about 725, about 750, about 775, about 800, about 825, about 850, about875, about 900, about 925, about 950, about 975, about 1000, about 1100, about 1200, about 1300, about 1400, about 1500, about 1750, about 2000, about 2250, about 2500 or greater amino molecule residues, and any range derivable therein.

The following table may be useful in interpreting the amino acid and nucleic acid sequences contained herein.

Codon Table

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Trytophan | Trp | W | UGG | | | | | |
| Tyrosine | Tyr | Y | UAC | UAU | | | | |

As used herein, an “amino molecule” refers to any amino acid, amino acid derivative or amino acid mimic as would be known to one of ordinary skill in the art. In certain embodiments, the residues of the prootenaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Proteinaceous compositions may be made by any technique known to those of skill in the art, including the expression of proteins polypeptides or peptides through standard molecular biological techniques, the isolation of proteinaceus compounds from natural sources, or the chemical synthesis of proteinaceous materials. The nucleotide and protein, polypeptide and peptide sequences for various genes have been previously disclosed, and may be found at computerized databases known to those of ordinary skill in the art. One such database is the National Center for Biotechnology Information's Genbank and GenPept databases (http://www.ncbi.nlm.nih.gov/). The coding regions for these known genes may be amplified and/or expressed using the techniques disclosed herein or as would be know to those of ordinary skill in the art. Alternatively, various commercial preparations of proteins, polypeptides and peptides are known to those of skill in the art.

In certain embodiments a proteinaceous compound may be purified. Generally, “purified” will refer to a specific or protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

Proteins and peptides suitable for use in this invention may be autologous proteins or peptides, although the invention is clearly not limited to the use of such autologous proteins. As used herein, the term “autologous protein, polypeptide or peptide” refers to a protein, polypeptide or peptide which is derived or obtained from an organism. Organisms that may be used include, but are not limited to, a bovine, a reptilian, an amphibian, a piscine, a rodent, an avian, a canine, a feline, a fungal, a plant, or a prokaryotic organism, with a selected animal or human subject being preferred. The “autologous protein, polypeptide or peptide” may then be used as a component of a composition intended for application to the selected animal or human subject. In certain aspects, the autologous proteins or peptides are prepared, for example from whole plasma of the selected donor. The plasma is placed in tubes and placed in a freezer at about −80° C. for at least about 12 hours and then centrifuged at about 12,000 times g for about 15 minutes to obtain the precipitate. The precipitate, such as fibrinogen may be stored for up to about one year (Oz, et al., 1990).

I. Screening For Modulators Of DC-SIGN Function

The present invention further comprises methods for identifying modulators of the function of the DC-SIGN isoforms 1, 2 and 3. These assays may comprise random screening of large libraries of candidate substances; alternatively, the assays may be used to focus on particular classes of compounds selected with an eye towards structural attributes that are believed tomake them more likely to modulate the function of the DC-SIGN isoforms.

To identify a modulator of a DC-SIGN isoform, one generally will determine the function of DC-SIGN isoform in the presence and absence of the candidate substance, a modulator defined as any substance that alters function. For example, a method generally comprises: (a) providing a candidate modulator; (b) admixing the candidate modulator with an isolated compound or cell, or a suitable experimental animal; (c) measuring one or more characteristics of the compound, cell or animal in step (d); and (e) comparing the characteristic measured in step (c) with the characteristic of the compound, cell or animal in the absence of said candidate modulator, wherein a difference between the measured characteristics indicates that said canfidate modulator is, indeed, a modulator of the compound, cell or animal.

Assays may be conducted in cell free systems, in isolated cells, or in organisms induding trarsenic animals. It will, of course, be understood that all the screening methods of the present invention are useful in themselves notwithstanding the fact that effective candidates may not be found. The invention provides methods for screening for such candidates, not solely methods of finding them.
1. Modulalors

As used herein the term “candidate substance” refers to any molecule that may potentially inhibit or enhance a DC-SIGN isoform activity. The candidate substance may be a protein or fragment thereof, a small molecule, or even a nucleic acid molecule. It may prove to be the case that the most useful pharmacological compounds will be compounds that are structurally related to the DC-SIGN isoforms. Using lead compounds to help develop improved compounds is know as “rational drug design” and includes not only comparisons with know inhibitors and activators, but predictions relating to the structure of target molecules.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.
2. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads. A technique for high throughput screening of compounds is described in WO 84/03564.
3. In Cyto Assays

The present invention also contemplates the screening of compounds for their ability to modulate D-SIGN isoforms in cells. Various cell lines can be utilized for such screening assays, including cells specifically engineered for this purpose. Cells of particular utility include dendritic cells, other antigen presenting cells, T-cells, and other cells of the immune system. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.
4. In Vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and mondeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. the characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

J. Kits

Any of the compositions described herein may be comprised in a kit. In a non-limiting example, a DC-SIGN isoform, lipid, and/or additional agent, may be comprised in a kit. The kits will thus comprise, in suitable container means, a DC-SIGN1, DC-SIGN2 or DC-SIGN3 isoform. The components of the kits may be packaged either in aqueous media or in lyophilized form.

The container means of the kits will generally include at least one vial, test tube, flask, bottle, syringe or other container means, into which a component may be placed, and preferably, suitably aliquoted. When there are more than one component in the kit, the kit also will generally contain a second, third or other additional container into which the additional components may be separately placed. However, various combinations of components may be comprised in a vial. The kits of the present invention also will typically include a means for containing DC-SIGN, lipid, additional agent, and any other reagent containers in close confinement for commercial sale. Such containers may include injection or blow-molded plastic containers into which the desired vials are retained.

K. Abbreviations

The more frequent abbreviations used are: HIV, human immunodeficiency virus; DC, dendritic cell; DC-SIGN, DC-specific ICAM-3-grabbing nonintegrin; ICAM, intercellular adhesion molecule; PECAM, platelet-endothelial cell adhesion molecule; CCR, CC chemokine receptor, Ex, exon; Ab, antibody; TM, transmembrane; PBHP, peripheral blook hematopoietic progenitor cells; PBMC, peripheral blood mononuclear cells; contig, group of overlapping clones; IL, interleukin; PHA, phytohemagglutinin; RT, reverse transcriptase; PCR, polymerase chain reaction; EST, expressed sequence tag; PBS, phosphate-buffered saline; bp, base pair(s); kb, kilobase pair(s); sDC-SIGN, soluble DC-SIGN; m, membrane-associated DC-SIGN.

L. Brief Description of the Sequence Listings

Many of the DC-SIGN encoding nucleotide sequences of the present invention are available in the GenBank/EMBL Data Bank with accession numbers AY042221 through AY042240.

The following listing provides a correspondence of the SEQ ID Nos and their contents as designated elsewhere and throughout the application.

| | Genbank | Polypeptide/amino | | |
|---|---|---|---|---|
| Nucleic acid SEQ ID | Accession No. | acid SEQ ID | | |
| NO: | suffix | NO: | DC-SIGN isoform designation | Other designation |
| 1 | 42221 | 2 | mDC-SIGN1A type I | |
| 3 | 42222 | 4 | mDC-SIGN1A type II | |
| 5 | 42223 | 6 | mDC-SIGN1A type III | |
| 7 | 42224 | 8 | mDC-SIGN1A type IV | |
| 9 | 42225 | 10 | sDC-SIGN1A type I | |
| 11 | 42226 | 12 | sDC-SIGN1A type II | |
| 13 | 42227 | 14 | sDC-SIGN1A type IIII | |
| 15 | 42228 | 16 | sDC-SIGN1A type IV | |
| 17 | 42229 | 18 | mDC-SIGN1B type I | |
| 19 | 42230 | 20 | sDC-SIGN1B type I | |
| 21 | 42231 | 22 | sDC-SIGN1B type II | |
| 23 | 42232 | 24 | sDC-SIGN1B type III | |
| 25 | 42233 | 26 | sDC-SIGN1B type IV | |
| 27 | 42234 | 28 | mDC-SIGN2 type I | |
| 29 | 42235 | 30 | mDC-SIGN2 type III | |
| 31 | 42236 | 32 | mDC-SIGN2 type IV | |
| 33 | 42237 | 34 | mDC-SIGN2 type VI | |
| 35 | 42238 | 36 | sDC-SIGN2 type I | |
| 37 | 42239 | 38 | sDC-SIGN2 type II | |
| 39 | 42240 | 40 | SDC-SIGN2 type III | |
| 41 | | | | Sense primer 1-1 |
| 42 | | | | Antisense primer 1-2 |
| 43 | | | | Sense primer 1-3 |
| 44 | | | DC-SIGN1 Exon Ib oligonucleotide | |
| 45 | | | DC-SIGN1 Exon Ic oligonucleotide | |
| 46 | | | DC-SIGN1 Exon II oligonucleotide | |
| 47 | | | DC-SIGN1 Exon Ic-Exon III | |
| | | | oligonucleotide | |
| 48 | | | DC-SIGN1 Exon IV oligonucleotide | |
| 49 | | | DC-SIGN2 cDNA PCR primer | |
| | | | sense 2-1 | |
| 50 | | | DC-SIGN2 cDNA PCR primer | |
| | | | antisense 2-2 | |
| 51 | | | DC-SIGN2 cDNA PCR primer for | |
| | | | southern sense 2-3 | |
| 52 | | | DC-SIGN2 cDNA PCR primer for | |
| | | | Southern sense 2-4 | |
| 53 | | | DC-SIGN2 cDNA PCR primer for PAGE | |
| | | | sense 2-5 | |
| 54 | | | DC-SIGN2 oligonucleotide | |
| | | | for southern analysis Exon II 2-6 | |
| 55 | | | BeWoI DC-SIGN isoform | |
| 56 | | | BeWoIII DC-SIGN isoform | |
| 57 | | | BeWoII DC-SIGN isoform | |
| 58 | | | BeWoIV DC-SIGN isoform | |
| 59 | | | DC-SIGN2 | |
| 60 | | 61 | DC-SIGN2 | |
| | | | Δ repeat 5 | |
| 62 | | 63 | DC-SIGN2 | |
| | | | Δ repeats 5-6 | |
| 64 | | 65 | DC-SIGN2 | |
| | | | Δ repeats 5-6/Δ exon 3/intron 4 | |
| | | | retension | |
| 66 | | 67 | DC-SIGN2 | |
| | | | Δ exon 3 and 6 | |
| 68 | | 69 | DC-SIGN2 | |
| | | | Δ exon 3/Δ repeat 5 | |
| 70 | | 71 | DC-SIGN2 | |
| | | | Δ exon 3/intron 4 retention | |
| 72 | | 73 | DC-SIGN2 | |
| | | | Δ exon 3/Δ repeats 5 and 6 | |
| 74 | | 75 | DC-SIGN2 | |
| | | | Δ half exon 2/Δ exons 3-5/Δ 6 aa | |
| | | | exon 6 | |
| 76 | | 77 | DC-SIGN2 | |
| | | | Δ exons 2 and 3/intron 4 retension | |
| 78 | | 79 | DC-SIGN2 | |
| | | | Δ exons 2, 3 and 9/Δ repeats 5 and | |
| | | | 6 | |
| 80 | | 81 | DC-SIGN2 | |
| | | | Δ exons 2, 3 and 6 | |
| 82 | | 83 | DC-S1GN2 | |
| | | | Δ exons 2 and 3 | |
| 84 | | 85 | DC-SIGN2 | |
| | | | Δ half exon 1/ | |
| | | | Δ exons 2, 3 and 4/Δ 8 aa exon 5 | |
| 86 | | 87 | DC-SIGN3 | |
| 88 | XM_064898 | | DC-SIGN3 | |
| | | | predicted mRNA | |
| 89 | BC39679 | | DC-SIGN3 cDNA | |
| 90 | BI755445 | | DC-SIGN3 | |
| | | | cDNA | |
| 91 | BI757731 | | DC-SIGN3 | |
| | | | cDNA | |
| | | 92 | | DC-SIGN1 |
| | | | | synthetic peptide |

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate tht many changes can be made in the specific embodiments which are disclosed and still obtain a like or similar result without departing from the spirit and scope of the invention.

### A. Example 1

### Materials and Methods

Cells, Cytokine Differentiation of DCs, and RNA. CD34+ peripheral hematopoietic progenitor cells (PBHP) and peripheral blook mononuclear cells (PBMCs) were isolated from healthy adult normal volunteers treated with granulocyte colony-stimulating factor (G-CSF, Amgen, Calif.) as described previously (Ahuja et al., 1998). The CD34+ PBHP cells were cultured in medium supplemented with 20 ng/ml of stem cell factor and 50 ng/ml granulocyte-macrophage colony-stimulating factor (R&D Systems, Minneapolis, Minn.). Tumor necrosis factor-(10 ng/ml) was added on day 7, and on day 11 of culture IL-4 (10 ng/ml) was added to one-half of the cells. The cytokine-differentiated CD34+ PBHP cells were kept in culture for a total of 15 days. By day 14 of culture more than 99% of cells were DC33+ indicating that the predominant cell population was of the myeloid series (Ahuja et al., 1998). The proportion of cells that stained for T/B lymphocyte markers (CD3/CD19) was less than 1-3%. PBMCs were also isolated from 20 ml of blood obtained from normal donors who did not receive granulocyte colony-stimulating factor. An aliquot of these PBMCs wre stimulated with PHA (5 Âμg/ml, Sigma) for 4 days. In some experiments IL-2 (50 units/ml, Life Technologies) was added to the culture medium after day 4. CD3 and CD28 monoclonal antibodies (PharMingen) were coated on tosyl-activated Dynal beads (Dynal, Lake Success, N.Y.) and used to stimulate PBMCs (1:1 concentration). The placenta samples were from anonymous normal donors. MRNA from highly purified leukocyte subsets, including CD14+ monocytes, was also obtained from a commercial source (CLONTECH). Cell lines were obtained from ATCC and the National Institutes of Health AIDS repository. Total RNA was extracted from cells using TrizolÂ® reagent (Life Technologies, Inc.) and first strand cDNA was generated using reverse transcriptase (RT) and random hexamens or oligo(dT) primers (Superscript TM Preamplification System, Life Technologies). The local institutional review board approved the studies conducted.

Primers, PCR Amplification, and Sequencing. The sequences of the oligonucleotides used in PCR and for hybridization experiments are shown in Table I. The cycling condition for PCR amplification of DC-SIGN1 cDNAs was 94 Â° C. for 10 s, 52 Â° C. for 30 s, and 72 Â° C. for 60 s. The cycling condition for amplification of DC-SIGN2 cDNAs was 94 Â° C. for 30 s, and 65 Â° C. for 30 s, and 72 Â° C. for 90 s. A total of 35 cycles was used. The PCR products were cloned into TOPO vectors 2.1 or II (Invitrogen) and sequenced on both strands. To determine the genomic structure of DC-SIGN1, a series of sense and antisense orientation primers based on the cDNA sequence described by Curtis et al. (1992) were designed (sequences not shown). Two expressed sequence tags (ESTs) that had homology to DC-SIGN2 were purchased from Research Genetics (Huntsville, Ala., Image Clones 146996 and 240697) and sequenced on both strands.

Oligonucleotides used in this study S, sense; AS, antisense; Ex, exon (location of the oligomer or orientation). SEQ ID Nos corresponding to these oligonucleotides are identified in the section entitled “Brief Description of Sequence Listing,” above.

TABLE I

Oligonucleotides used in this study
S, sense; AS, antisense; Ex, exon (location of the
oligomer or orientation).

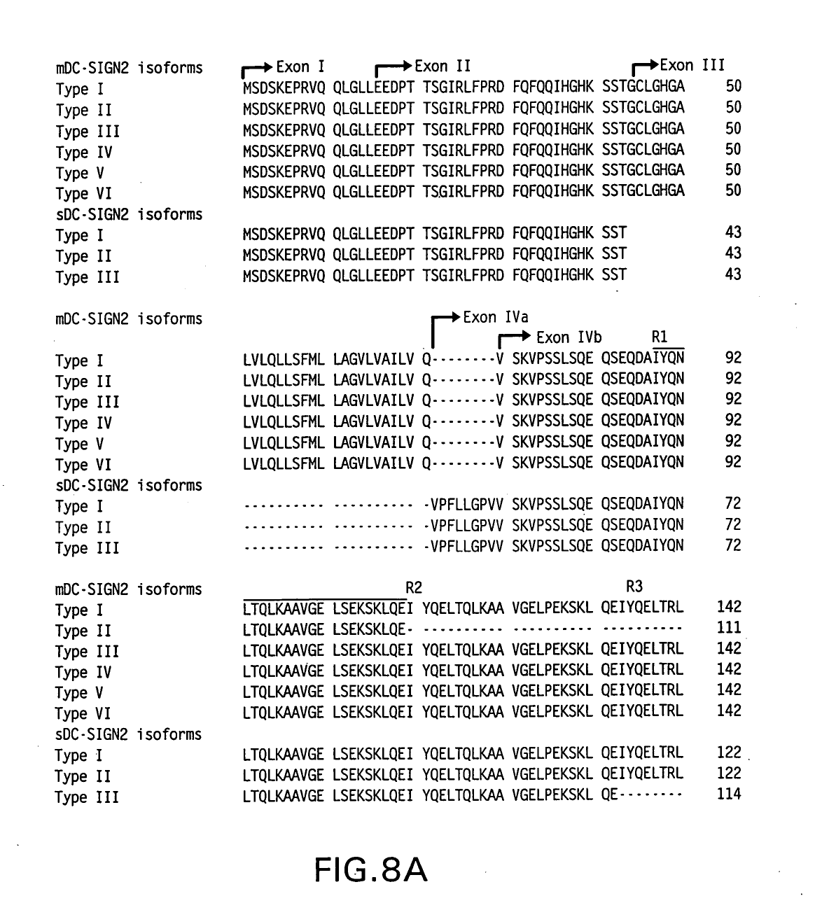

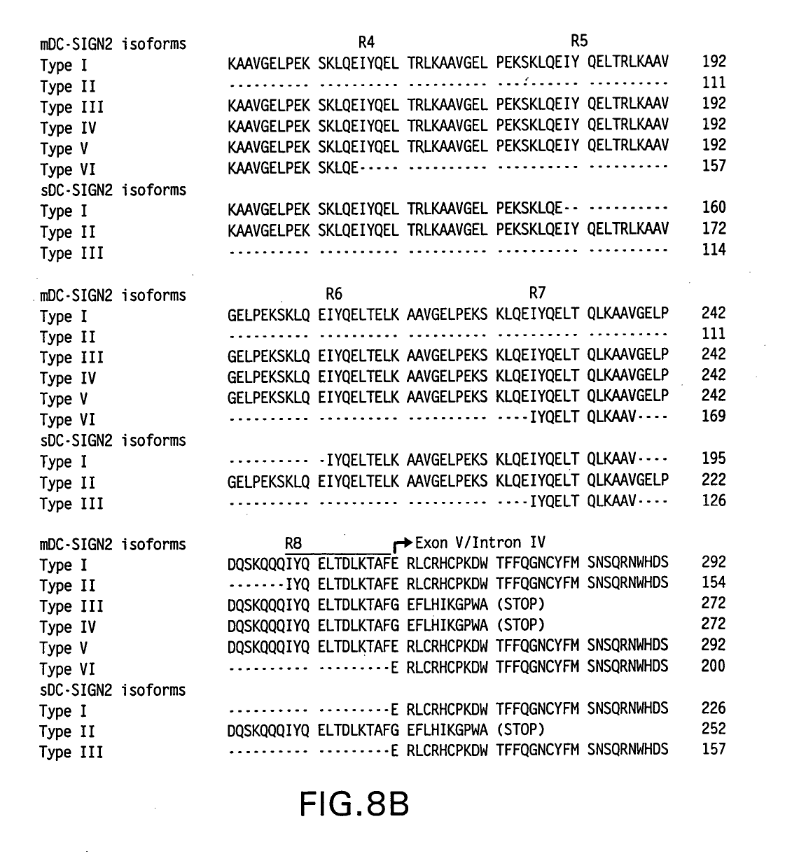

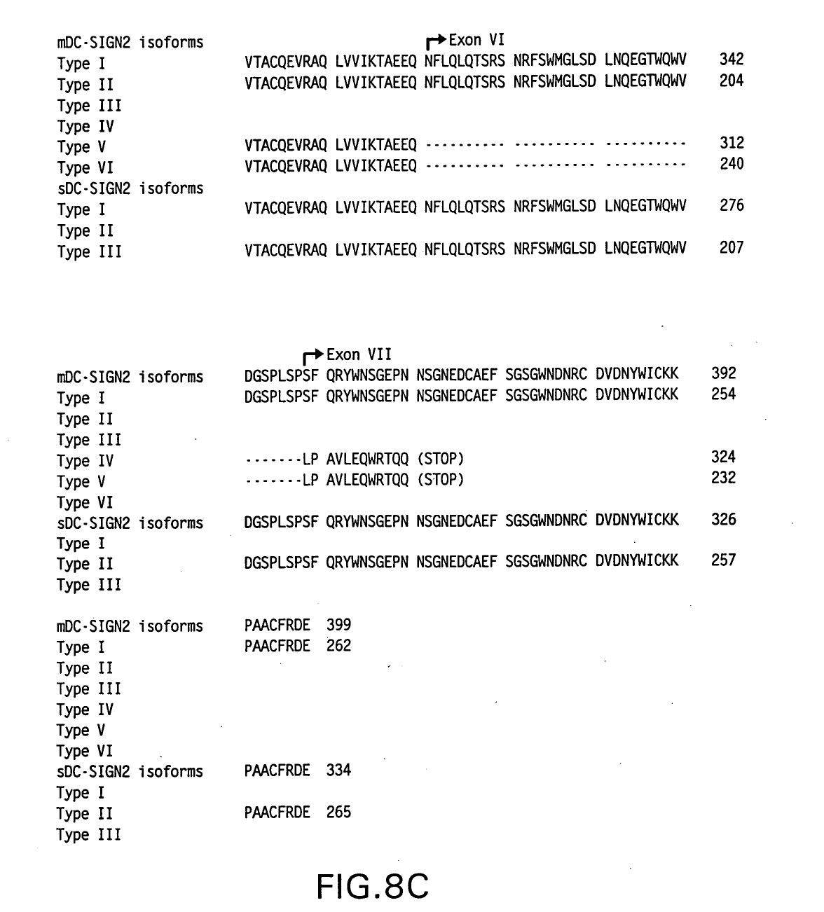

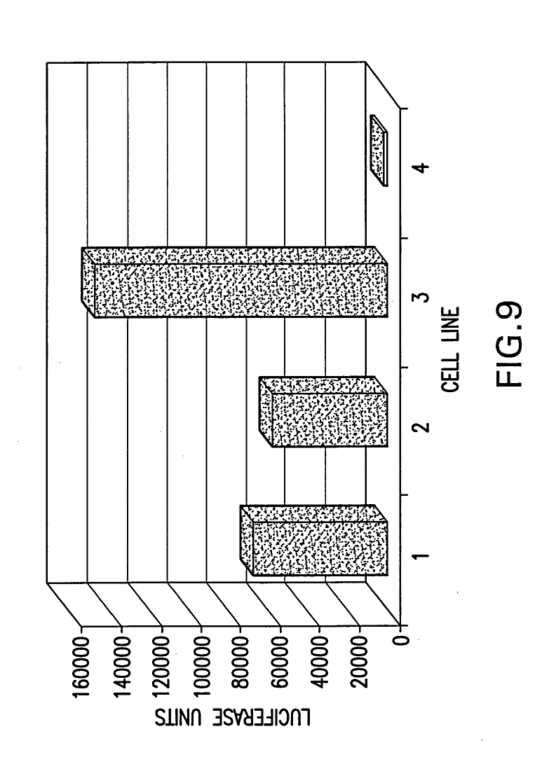

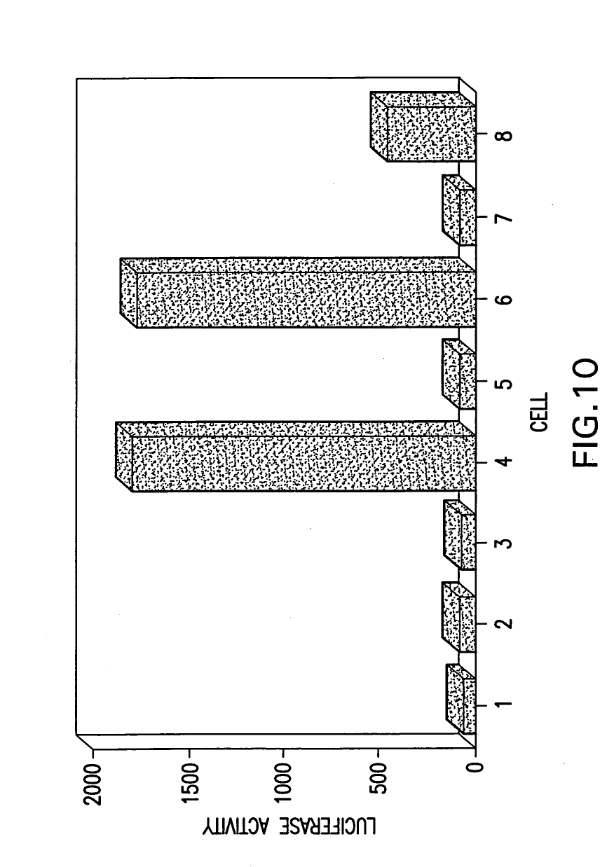

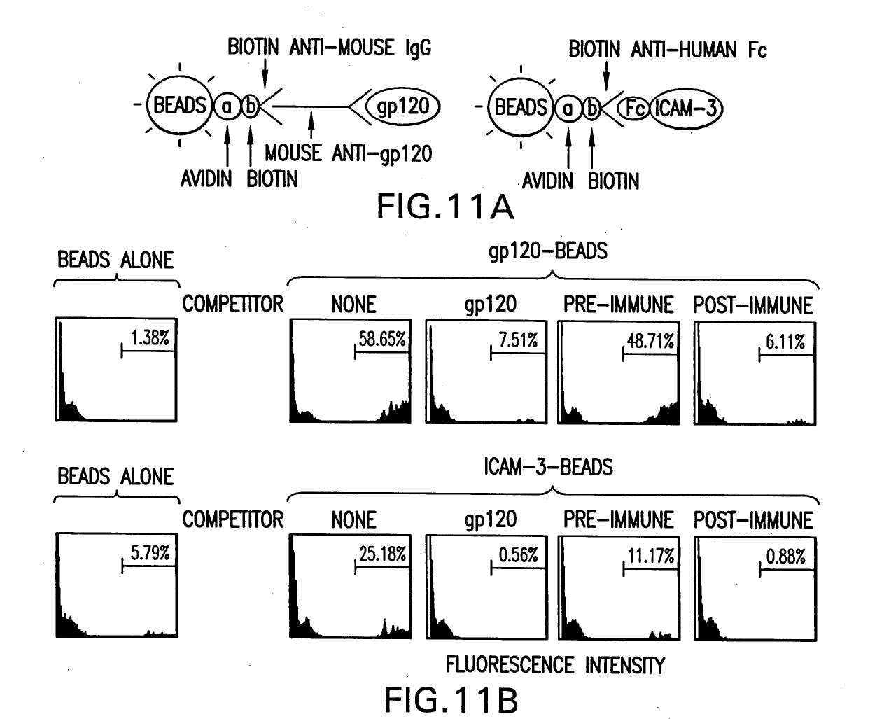

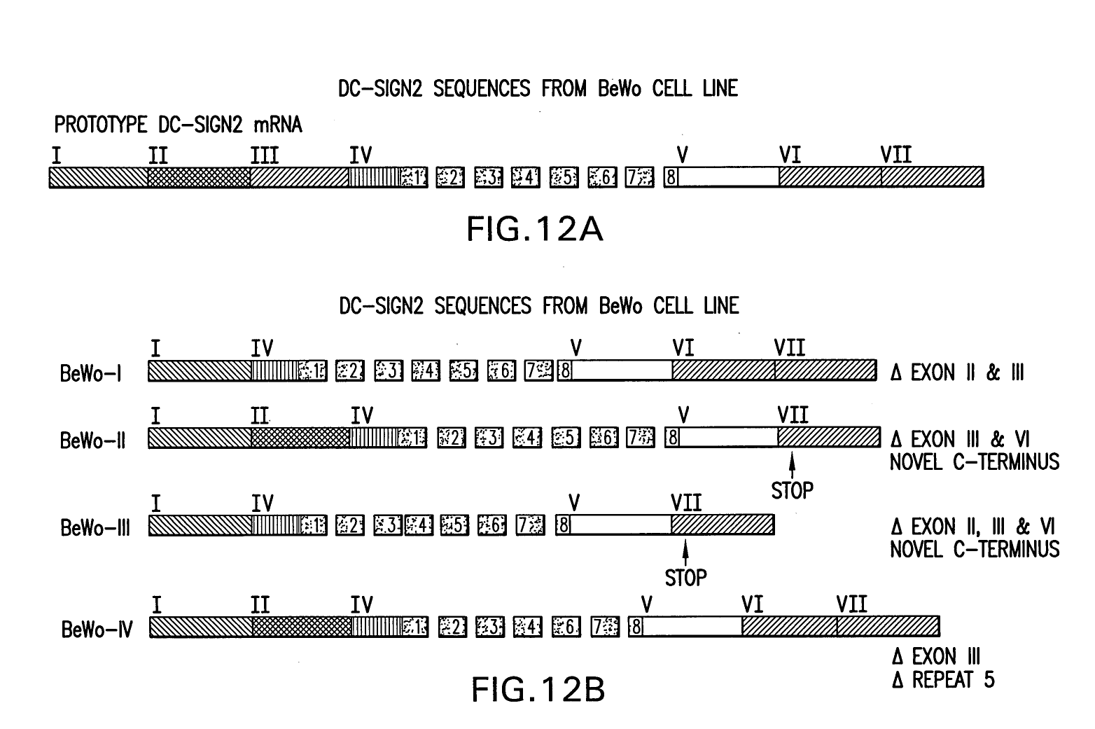

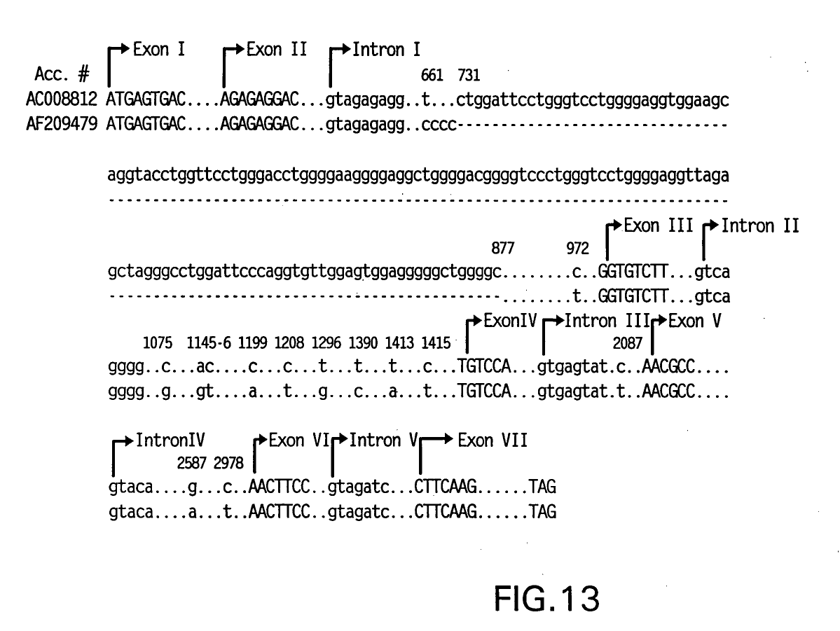

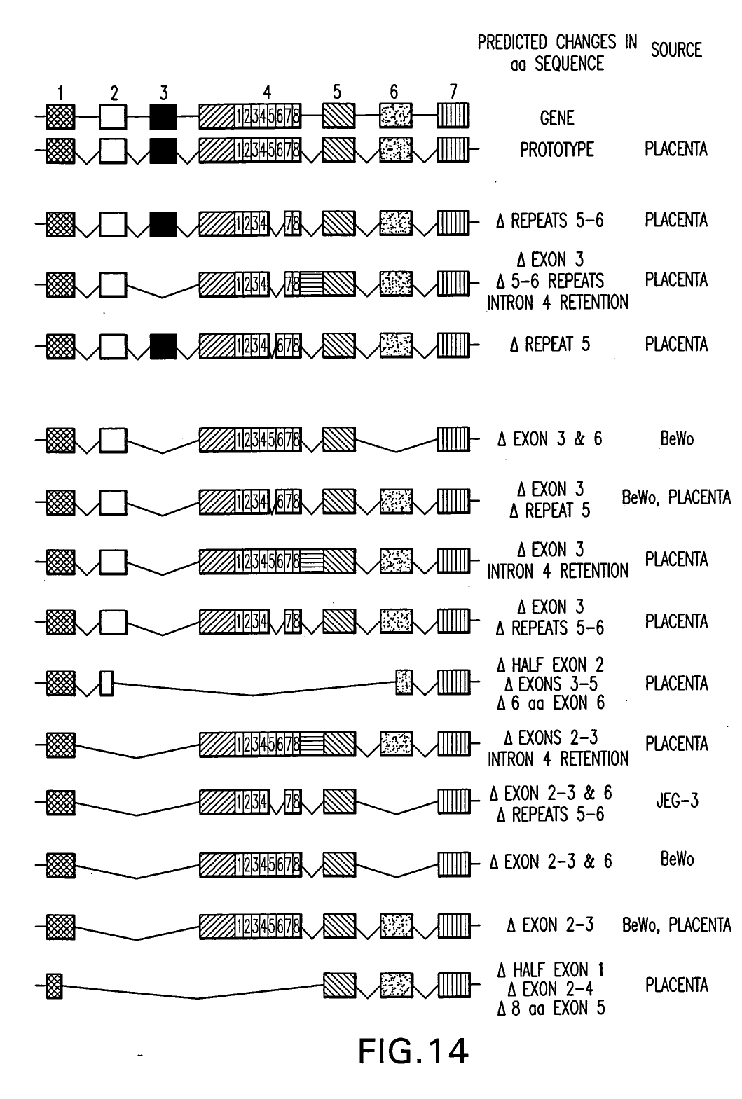

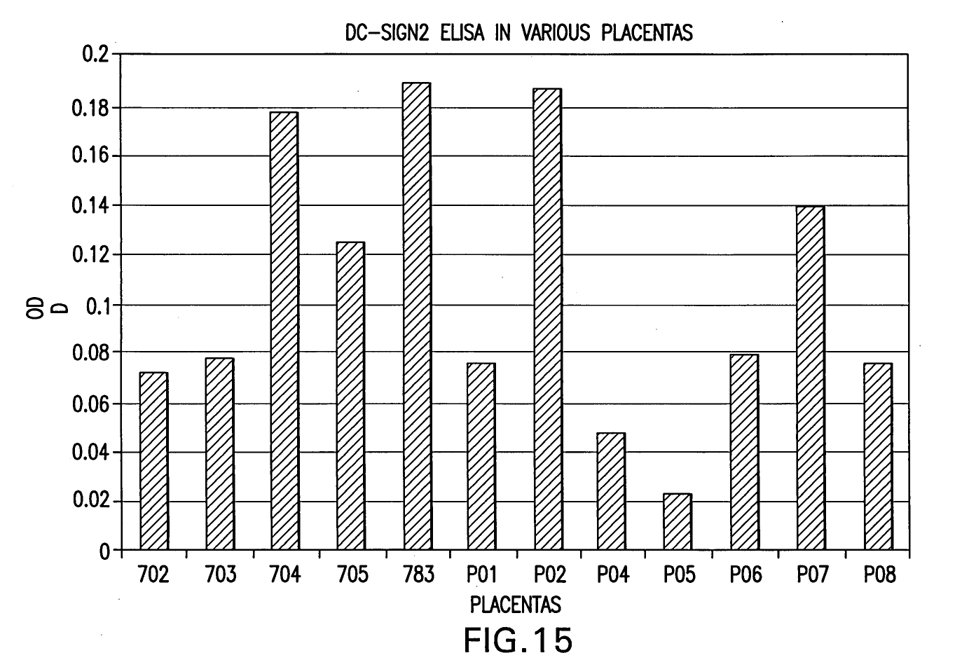

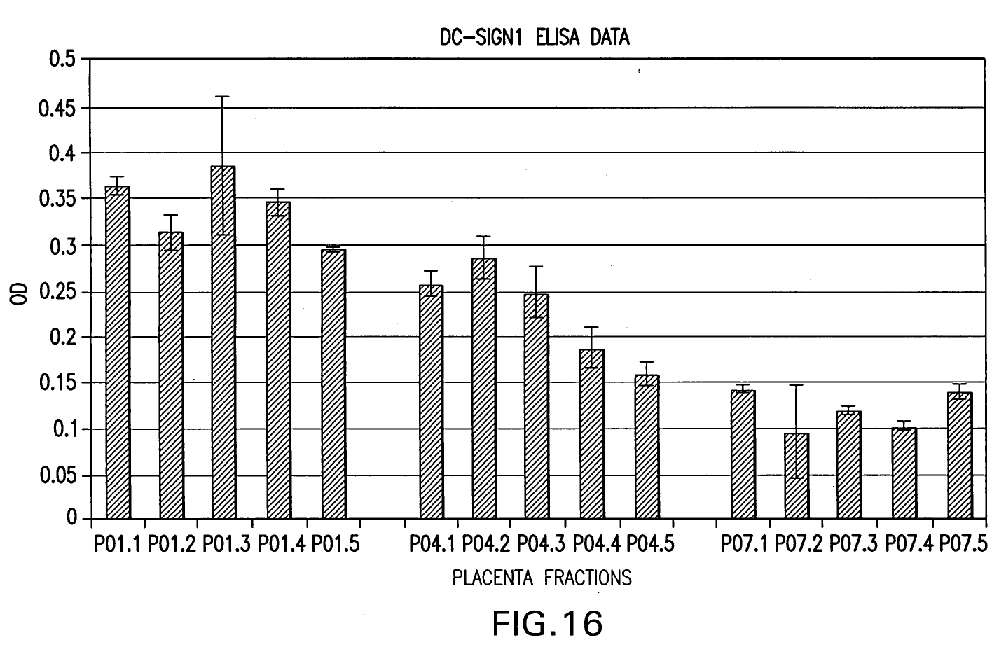

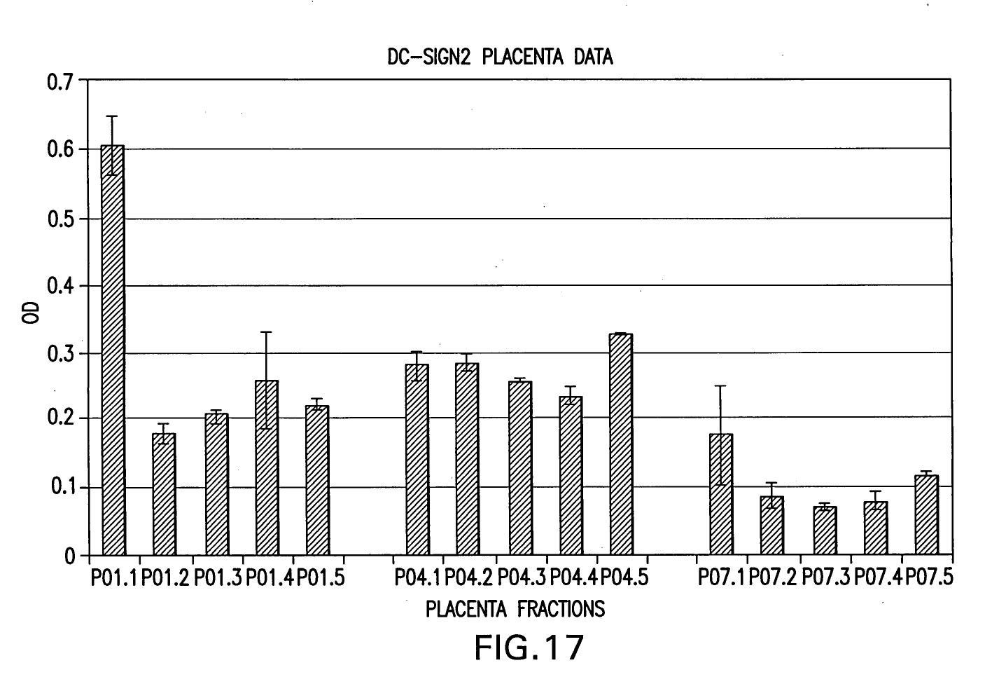

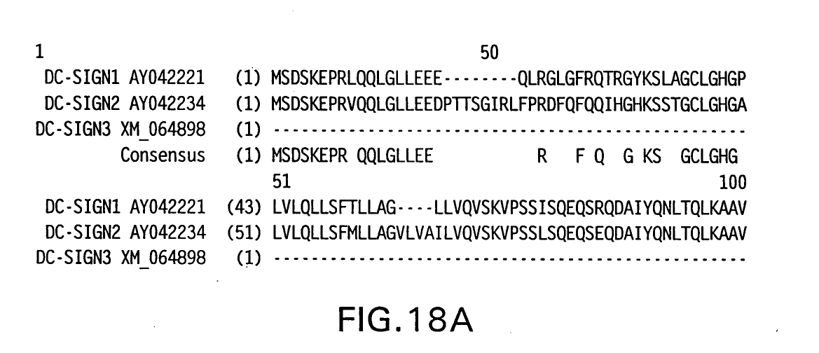

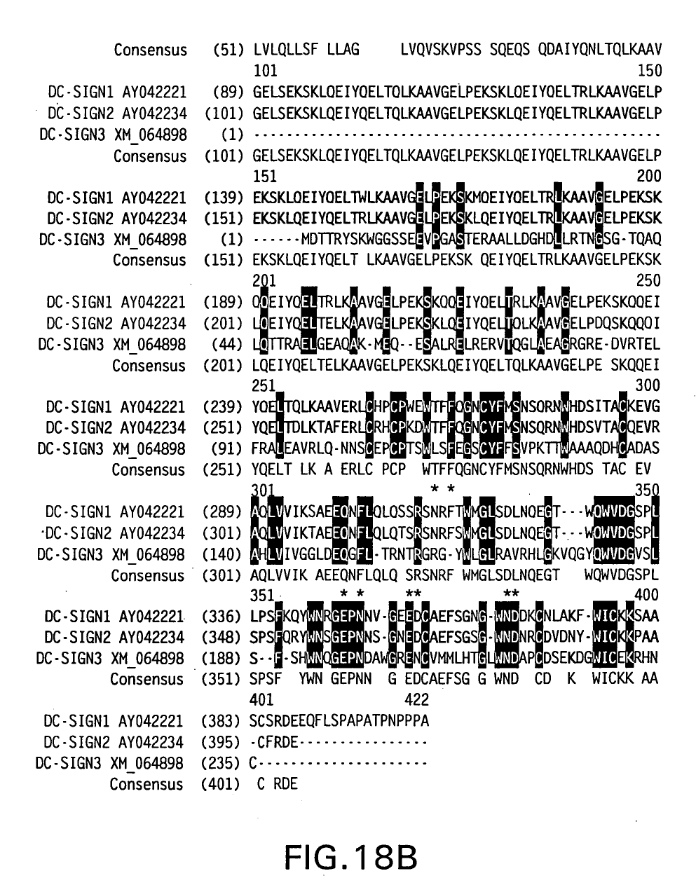

PCR primers used for DC-SIGN1 cDNA cloning (FIG. 1),
Southern blot analysis (FIGS. 8 and 9), and
PAGE analysis (FIG. 11)

| | | |
|---|---|---|
| 1-1 | atg agt gac tcc aag gaa | (S) |
| 1-2 | aag cta cag ttc ctt ctc tcc | (AS) |
| 1-3 | gtg agg ctg ggt tgg gac gct | (S) |

DC-SIGN1 oligonucleotides used for Southern blot analysis
(FIGS. 8 and 9 and data not shown)

| | | |
|---|---|---|
| 1-4 | ggc ctc agc ctg ccc agg ctc | (Ex Ib) |
| 1-5 | agg aac agc tga gag gcc ttg | (Ex Ic) |
| 1-6 | tct tgg ctg ggc tcc ttg tcc aag | (Ex II) |
| 1-7 | gag ctt agc agt gtc caa ggt c | (Ex Ic-Ex III) |
| 1-8 | ctt caa gca gta ttg gaa cag agg aga gc | (Ex VI) |

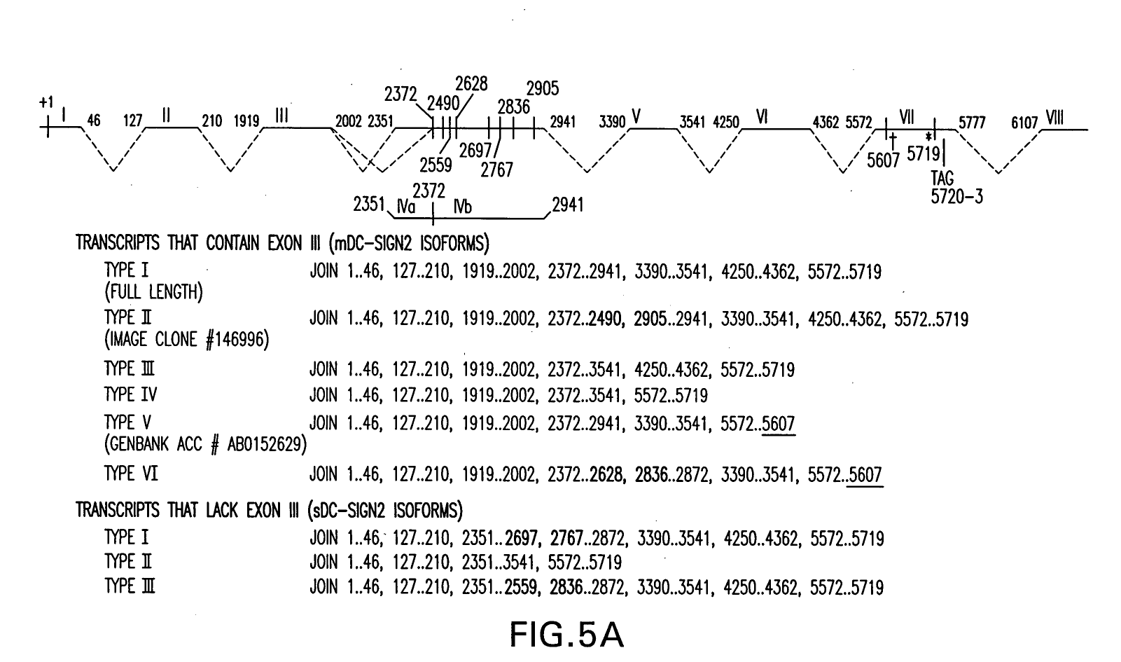

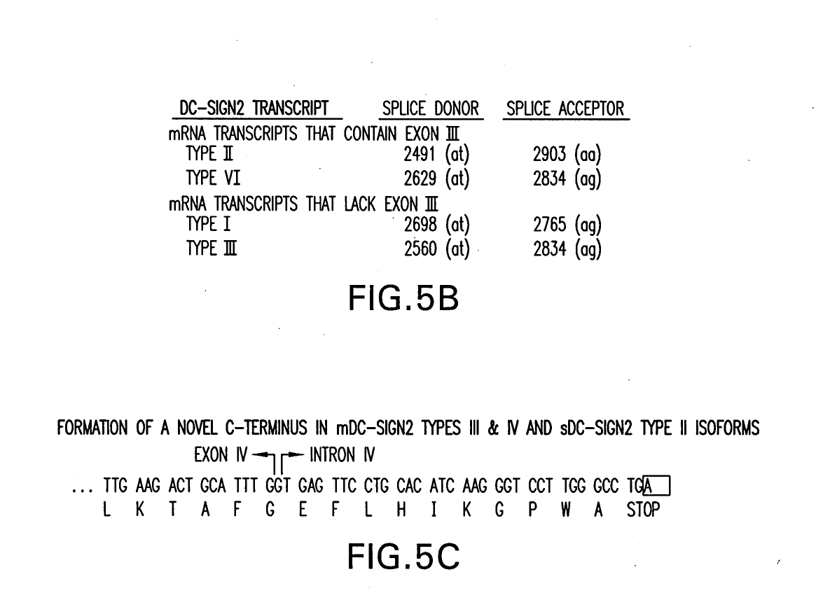

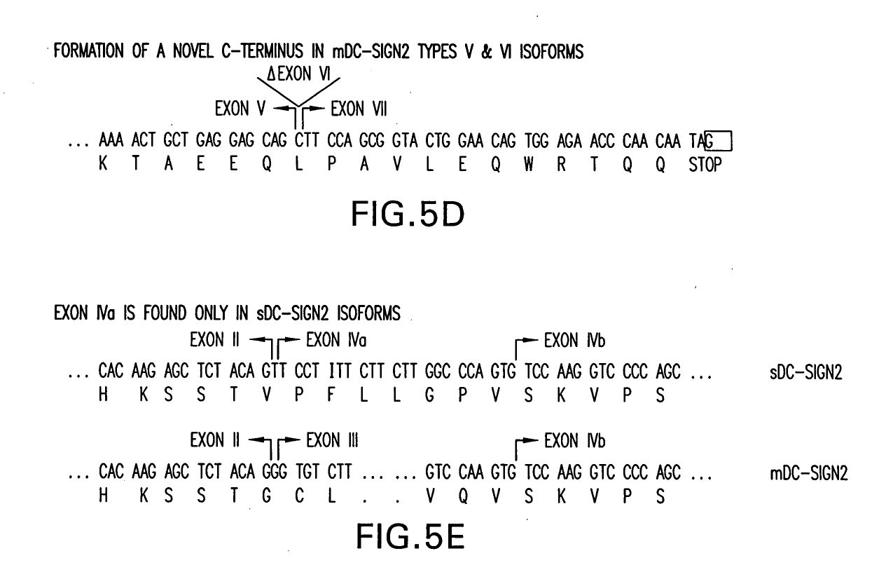

PCR primers used in generating DC-SIGN2 cDNAs (FIG. 5)

| | | |
|---|---|---|
| 2-1 | ggt acc aac atc tgg gga cag cgg gaa aac atg | (S) |
| 2-2 | gct cta gac tat tcg tct ctg aag c | (AS) |

PCR primers used in generating DC-SIGN2 cDNA for
Southern blot analysis (data not shown)

| | | |
|---|---|---|
| 2-3 | atc tgg gga cag cgg gaa | (S) |
| 2-4 | tgg agt gaa gaa gcg ctg | (AS) |

PCR primers used in generating DC-SIGN2 cDNA for PAGE
analysis (FIG. 12)

| | | |
|---|---|---|
| 1-1 | atg agt gac tcc aag gaa | (S) |
| 2-5 | gga ggc tga ggc tag cag | (AS) |

DC-SIGN2 oligonucleotide used for Southern blot analysis
(data not shown)

| | | |
|---|---|---|
| 2-6 | aag atc caa caa cca gtg gca | (Ex II) |

Southern Blot Hybridization. One Âμg of total RNA was used for synthesizing cDNA by random primers (Superscript Preamplification System, Life Technologies, Inc.). One-tenth of the cDNA product was used for PCR amplification. The PCR amplification profile consisted of 30 cycles of 94 Â° C. for 10 s, 55 Â° C. for 30 s, and 72 Â° C. for 60 s. PCR amplification was performed in a 100-Âμl reaction volume in the presence of 20 mM Tris-HCl, 40 mM KCl, 1.5 mM MgCl2, 0.1 mM of each dNTP, 0.2 ÂμM of each primer, and 2.5 units of Taq DNA polymerase (Life Technologies, Inc.). The primers used for amplification were oligonucleotides 1-1 and 1-2 for DC-SIGN1 and oligonucleotides 2-3 and 2-4 for DC-SIGN2 (Table I). An oligonucleotide that is DC-SIGN1 exon Ib-specific (oligonucleotide 1-3, Table I) was used to amplify exon Ib-containing cDNAs. The amplified products were size-fractionated by electrophoresis on a 1.5% agarose gel. After denaturation in alkaline solution, the DNA was transferred to a nylon membrane (Amersham Pharmacia Biotech) by capillary action. Hybidization was performed with the following end-labeled oligonucleotide probes: (i) oligonucleotides derived from DC-SIGN1 sequences in exon Ib, exon Ic, exon II, and exon VI (oligonucleotides 1-4, 1-5, 1-6, and 1-8, respectively in Table I); (ii) an oligonucleotide that had identity with DC-SIGN2-specific exon II sequences (oligonucleotide 2-6, Table I). the membranes were hybridized with the radiolabeled probes at 42 Â° C. for 12 h and washed under the following conditions: 2Â—SSC, 0.1% SDS at 42 Â° C. for 5 min (twice); 0.1Â—SSC, 0.1% SDS at 45 Â° C. for 15 min (twice). The filters were exposed to Biomax (MR) film (Kodak) at 80 Â° C. in a Quanta III cassette for 15 h.

Polyacrylamide Gel Electrophoresis. DC-SIGN1 and DC-SIGN2 cDNAs were amplified using the PCR conditions described above in a 50-Âμl reaction. The primers used for amplification were oligonucleotides 1-1 and 1-2 for DC-SIGN1 and oligonucleotides 1-1 and 2-5 for DC-SIGN2 (Table I). One of the primers used for amplification was end-labeled with 32P to facilitate detection of the PCR products by autoradiography. Five \overline{A}μl of the PCR product was mixed with 15 Aμl of formamide dye (95% formamide, 10 mM EDTA, 0.02% bromphenol blue, 0.02% xylene cyanol) and boiled for 5 min. The mixture was then chilled and loaded on a 4 or 4% polyacrylamide gel containing 8 M urea and electrophoresed for 12 h at 200 V in a Protean II xi cell (Bio-Rad). The polyacrylamide gels were dried, and autoradiogrphy was performed as described above.

In Vitro Translation. The TNTÂ®-coupled Reticulocyte Lysate System (Promega) was used to translate in vitro DC-SIGn1 cDNAs cloned into pcDNA4/HisMax TOPO vector (Invitrogen). The 35S-labeled translated products were fractionated in a 9% acrylamide gel and were exposed to XAR-2 film (Kodak) in a Quanta III cassette.

Antibodies and Peptides. A synthetic peptide (NH2-CSRDEEQFLSPAPATPNPPPA-COOH) (SEQ ID NO:92) derived from the C-terminal region of DC-SIGN1 was KLH-conjugated and used to immunize rabbits. The corresponding peptide sequence is absent in the DC-SIGN2. Rabbits were bled after 6 weeks to obtain polyclonal antiserum and were subsequently affinity-prified. Goat polyclonal antibodies (Ab) for CCR5 (sc-6128), PECAM-1 (sc-1505), and the corresponding blocking peptides were purchased from Santa Cruz Biotechnology (Santa Cruz, Calif.). The DC-SIGN1 blocking peptide was synthesized by Zymed Laboratories Inc. (San Francisco, Calif.).

Immunohistochemistry. OCT (Sakura Finetek USA, Inc., Torrance, Calif.)-embedded frozen term placental sections were air-dried for 30 min, washed in PBS (pH 7.4, and fixed in 4% cold paraformaldehyde for 10 min. The fixed sections were washed in Tris-buffered saline for 5 min, and were permeabilized with 0.05% PBS-TweenÂ® (Sigma Chemical Co. St. Louis, Mo.) for 5 min. All of the subsequent washes were in PBS-TweenÂ®. the sections were blocked using an avidin-biotin blocking kit (Vector Laboratories, Burlingame, Calif.) according to the manufacturer's instructions. Subsequently the sections were blocked with 5% bovine serum albumin for 30 min, washed, and incubated with either DC-SIGN1 antiserum or DC-SIGN1 antiserum plus DC-SIGN1 blocking peptide for 1 h. The sections were washed for 5 min, incubated with a 1:100 dilution of biotinylated goat anti-rabbit antibody (Dako, Carpinteria, Calif.) for 30 min, washed, and then stained for 30 min with the avidin-biotin complex-glucose oxidase system (Vector Laboratories). Color development was achieved using the glucose oxidase substrate kit (Vector Laboratories). Distilled water was used to block additional color development. For double staining, the sections were incubated in PBS for 5 min, and endogenous peroxidases were inhibited using a peroxidase block (Santa Cruz) or 5 min. Sides were then washed in PBS-Tween for 5 min, blocked with 5% bovine serum albumin for 30 min, and then incubated with one of the following: (i) PECAM-1 Ab, (ii) PECAM-1 Ab and its blocking peptide, (iii) CCR5 Ab, or (iv) CCR5 Ab and its blocking peptide. Subsequent steps for detecion of goat primary antibldies was performed using the goat immunocruz staining system according to the manufacturer's directions (Santa Cruz). Sections were incubated with diaminobenzidine for 10 min, and the reaction was stopped with distilled water. the sections were then dehydrated with graded alcohols and two washes in xylene and were mounted with Vectamount™ (Vector Laboratories).

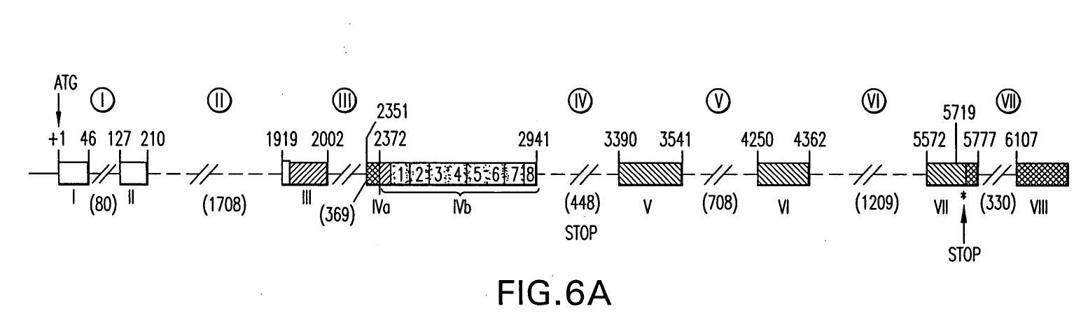

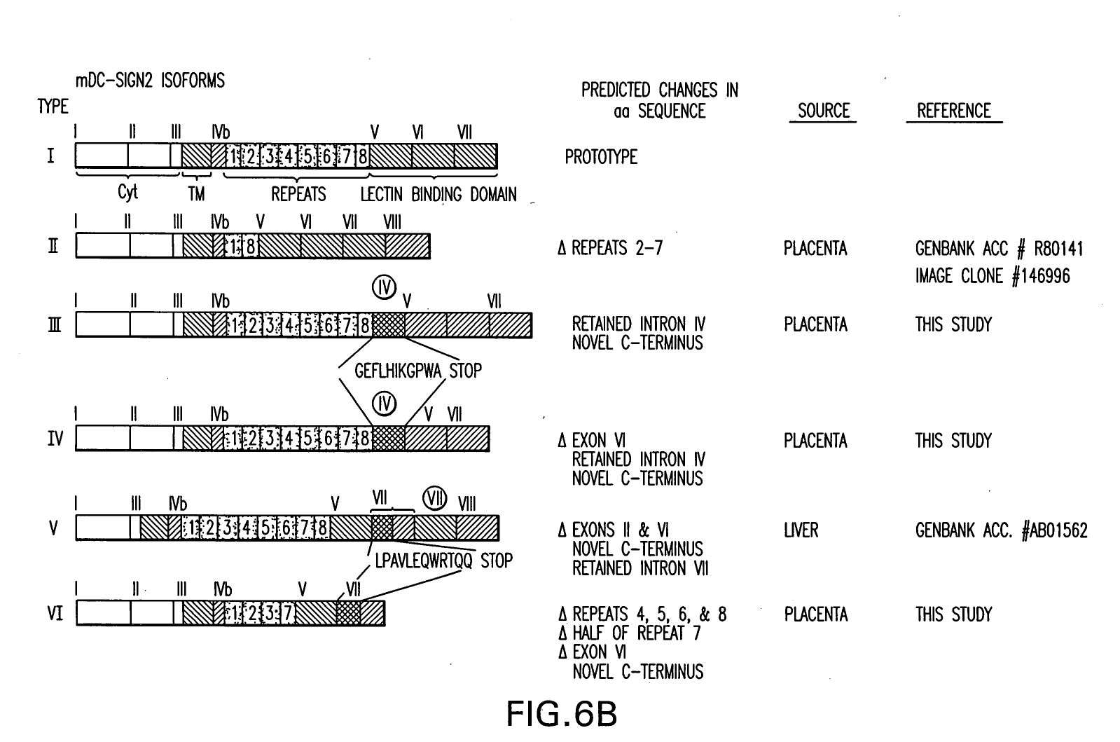

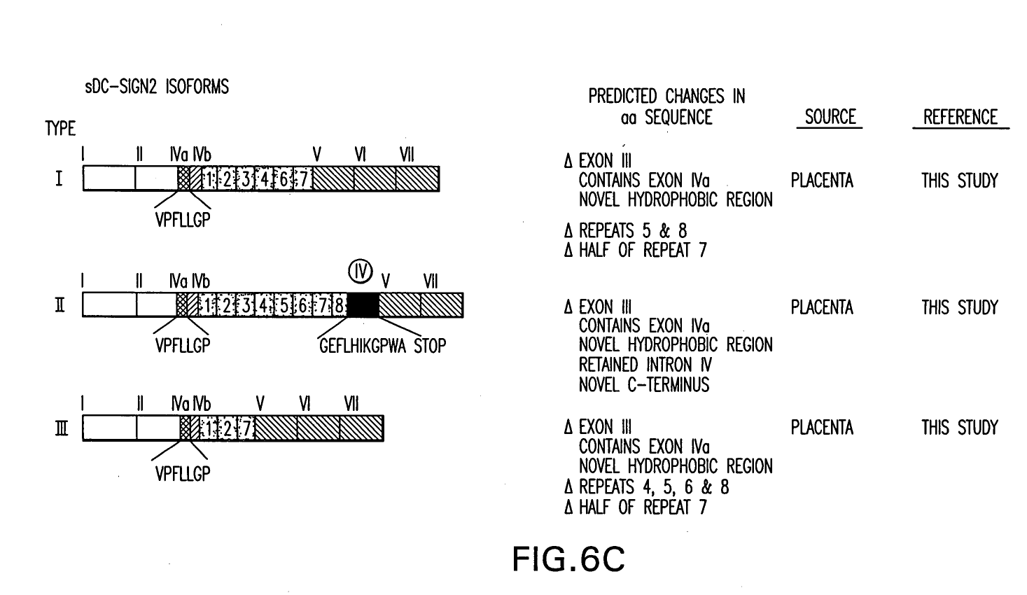

Generation of polyclonal anti-DC-SIGN1 antibodies. A peptide corresponding to the C-terminus of the polypeptide encoded by Cys384-Ala404 was used to generate a polyclnal antipeptide antiserum against DC-SIGN1. this is the same region used by Curtis et al to generate a polyclonal Ab against DC-SIGN1 (Curtis et al., 1992). Surface expression for DC-SIGn1-expressing THP-1 cell lines (NIH AIDS repository) could be detected using this antibody (FIG. 6), however, expression was not detected in non-transfected THP-1 cells (data not shown).

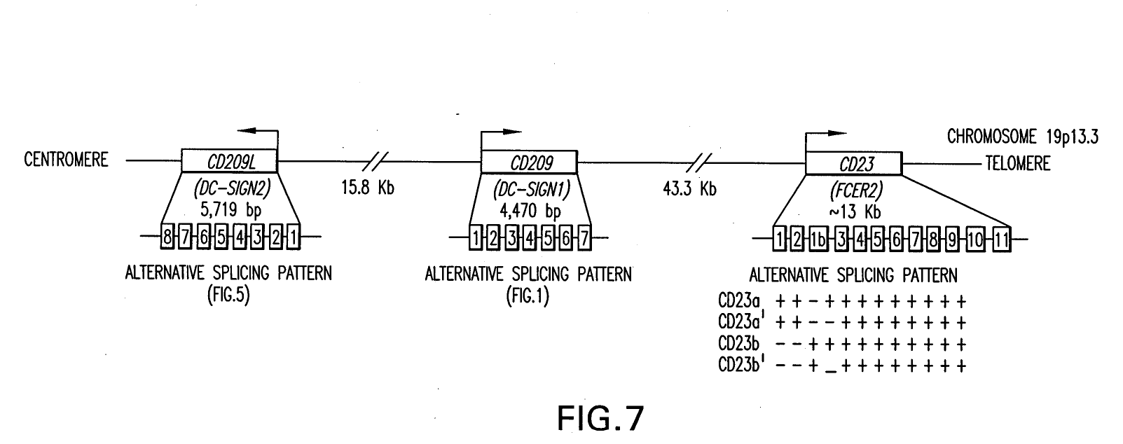

Development of a gp120 and ICAM-3 b adhesion assay. the inventors adopted the previously published bead-based strategy (Geijtenbeek et al., 2000; Geijtenbeek et al., 2000a; 2000b) to determine gp120 or ICAM-3 binding to DC-SIGN1-expressing cell lines (FIG. 7) or primary cells (data not shown). GP-120 adhesivity to DC-SIGN1-expressing cells could be inhibited by free gp120 (20 μg/ml) and post-immune sera (polyclonal Ab; 100 dilution) but not preimmune (1:100 dilution) sera. ICAM-3 adhesivity to DC-SIGN1-expressing cells could be completely inhibited by unlabeled gp120, suggesting that the binding sites for gp120 and ICAM-3 overlap. The preimmune sera also reduced ICAM-3 binding non-specifically from ˜25% to ˜11% (this non-specific blocking was not observed for gp120 binding), whereas the post-immune sera completely abolished ICAM-3 binding. (In these assays, 20 beads/cell were used).

Growth of DCs. The inventors have had a long-standing interest in DC biology and extensive expertise in the growth of human and murine immature (iDCs) and mature (mDCs) DCs from CD34+ progenitors as well as PBMCs (Ahuja et al., 1998; Ahuja et al, 1999; Ahuja, 2001; Quinones et al., 2000; Sato et al., 2000)) The inventors have explored several different methods for growing DCs from monocytes, and the protocol that they follow is an adaptation of that used by several investigators in the DC field, and essentially results in a standard model of human myeloid dendritic cells (Banchereau and Steinman, 1998; Jonuleit et al., 1997; Hart, 1997). In this model, iDCs are CD83-negative (lack of expession of CD80, CD83, intermediate epression of HLA class 1, HLA-DR, CD1a, CD40, CD54 and CD86) whereas MDCs are CD83-positive (high levels of HLA class I, HLA-DR, CD40, CD54, CD80, and CD86).

Briefly, PBMCs are isolated using lmphocyte separation medium (ICN biomedicals), washed twice in PBS and once with PBS spplemented with BSA (0.5%) and EDTA (2.0 mM) (PBSE). The cells are resuspended in PBSE at 5×10⁸ cell/ml at 4° C., combined with CD14-specific immunomagnetic reagent (Microbeads, Mittenyi Biotec; 200 ul of beads/4×10⁸ PBMC) and incubated at 6 C. for 15 min. Cells are magnetically purified according to the manufacturer's protocol (MACS, Mittenyi Biotec). Isolated cells are counted, and assayed for viability. On day 0, CD14+ cell are plated at 2×10⁶/ml in six-well plaltes in X-VIVO 15 medium (Bio-Whittaker) supplmented with human AB serum (1.0%; Sigma), penicillin/streptomycin, GM-CSF (800 U/ml) and IL-4 (1000 U/ml). On day 3 and day 5, each well is supplemented with 1 ml of this medium with GM-CSF concentration increased to 1600 U/ml. On day 7, non-adherent cells is collected by pipetting. To obtain MDCs, iDCs (1.0×10⁶ cells/ml) are cultured for three more days in Day 0 medium with addition of IL-6 (1,000 U/ml), TNF-a (1,100 U/ml), IL-10 (1,70 U/ml; all R & D), and prostaglandin E2 (PGE2; 1.0 ug/ml). After 3 days, non-adherent cells are collected by pipetting and processed as mDCs. The yield of mDC from iDC ranges from 60-70%.

### B. Example 2

### DC-SIGN1

In the course of identifying polymorphisms in DC-SIGN1, the inventors identified several alternatively spliced DC-SIGN1 cDNAs. To identify the genomic sequences homologous to these cDNAs, the inventors determined the gene structure for human DC-SIGN1. Genomic DNA was subjected to PCR using primers corresponding to the known cDNA sequence (Curtis et al., 1992); GenBank™ accession no. M98457), and the PCR products were cloned and sequenced. In addition, while this work was in progress, as part of the Human Genome Sequence Project, a ˜143,619-bp contig of human chromosome 19p that contained DC-SIGN1 became available (GenBank™ accession no. AC008812). Oter than a few polymorphisms, there was complete homology between the DC-SIGN1 genomic sequences that the inventors had identified and those found in this contig (data not shown). Comparisons of the cDNA and genomic sequences revealed that the coding region of the previously described prototypic DC-SIGN1 cDNA (GenBank™ accession no. M98457) was encoded by six exons (FIG. 1a, top panel). The nomenclature for the exons was based on the alternatively spliced exons identified in the DC-SIGN1 cDNAs (see below). Exons Ia and Ic encoded the majority of the cytoplasmic domain of the prototypic DC-SIGN1 cDNA (Curtis et al., 1992). Exon II encoded 5 amino acids of the cytoplasmic domain and the entire transmembrane (TM) doman. Exon III encoded the repeats as well as a short stretch of amino acids that preceded the seven full repeats and the one-half repeat. Exons IV, V, and VI together encoded the predicted extracellular lectin-binding domain of DC-SIGN1.

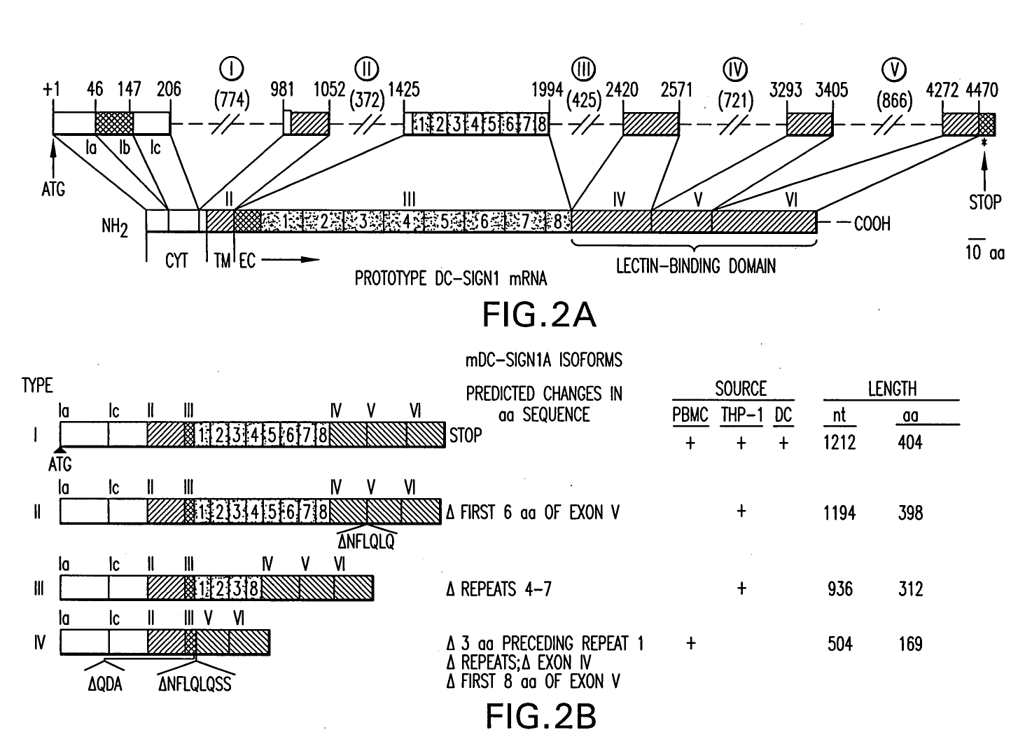

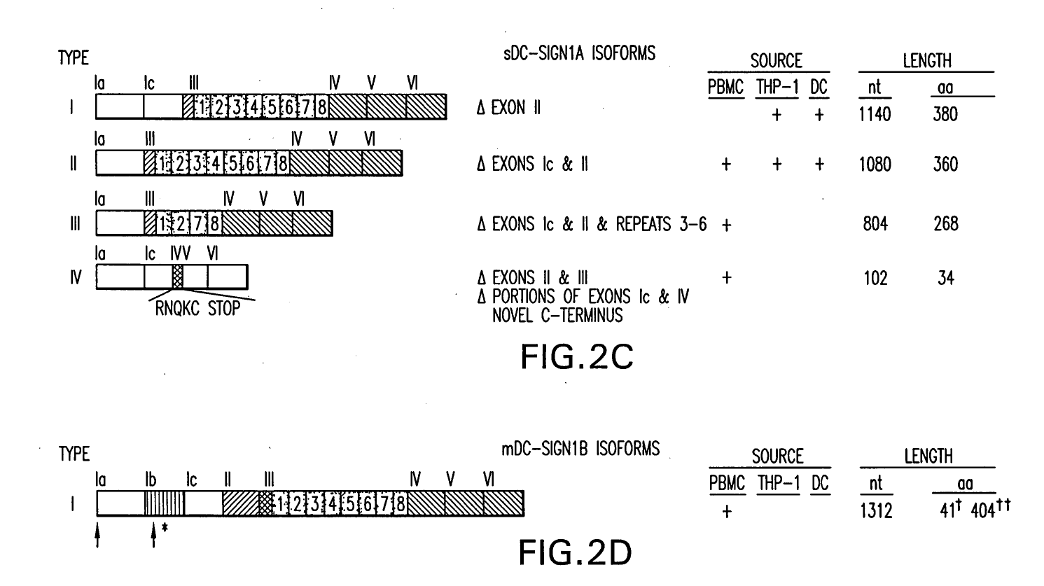

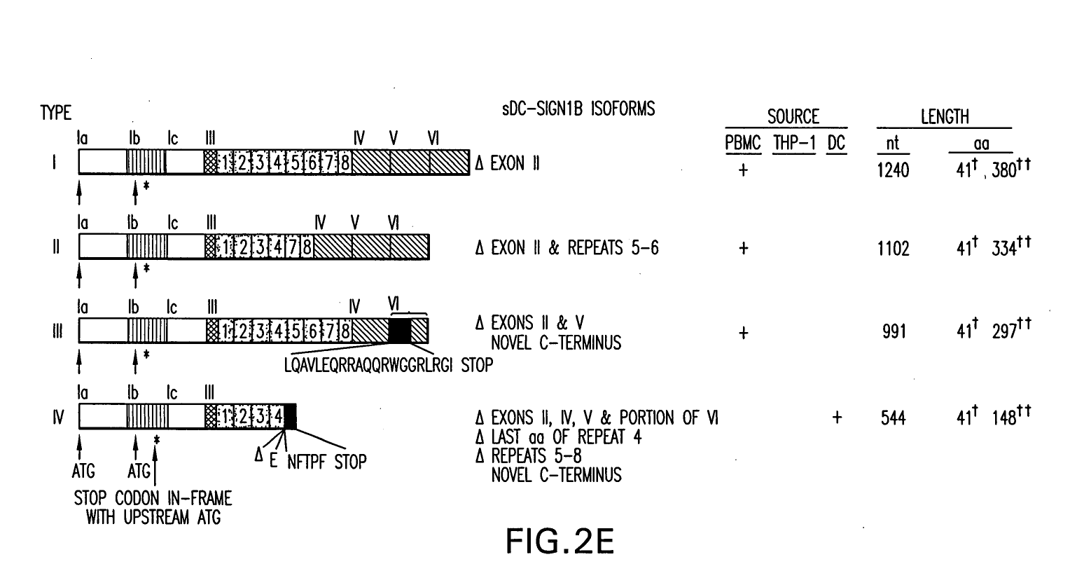

RT-PCR was used to amplify DC-SIGN1 cDNAs from PHA-activated PBMCs derived from normal human donors, human CD34+ PBHP-derived mature DCs, and THP-1 monocytic cells. Sequence analyses of these PCR amplicons revealed several distinct cDNAs that shared homology to the previously reported prototypic DC-SIGN1 cDNA (FlG. 1a and FIG. 1b; Curtis et al., 1992). These novel DC-SIGN1 transcripts differed from the originally reported cDNA sequence (GenBank™ accession no. M98457) by the presence or absence of stretches of sequences, indicating that they had arisen by a complex pattern of alternative splicing events in the exons encoding the intra- or extracellular domains and/or by splicing out of exon II, the exon that encodes the predicted TM domain (FIG. 1a and FIG. 1b). The predicted translaton products of these transcripts are illustrated in FIG. 2 and shown in Supplementary FIG. 1 and FIG. 2.

Based on the structures predicted from their amino acid sequences, the DC-SIGN1 isoforms could be categorized into one of five major groups (FIG. 1a; FIG. 1b; FIG. 2), namely mDC-SIGN1A, sDC-SIGN1A, mDC-SIGN1B, sDC-SIGN1B, and truncated DC-SIGN1 1B (tDC-SIGN1B). The first group of transcripts designated as membrane-associated or mDC-SIGN1A transcripts had a Met (ATG) translation initiation codon within exon Ia and retained the exon predicted to encode the TM domain (exon II; FIG. 1a; FIG. 2a; FIG. 2b). These transcripts included the prototypic DC-SIGN1, designated here as mDC-SIGN1A Type I, as well as additional transcripts that are predicted to encode variable portions of the extracellule domain (FIG. 1a; FIG. 2a; FIB. 2b). For example, in mDC-SIGN1A Type II, the first 6 amino acids encoded by exon V are spliced out, whereas in mDC-SIGN1A Type III, some of the repeats encoded by exon III are spliced out (FIG. 1a; FIG. 2b).

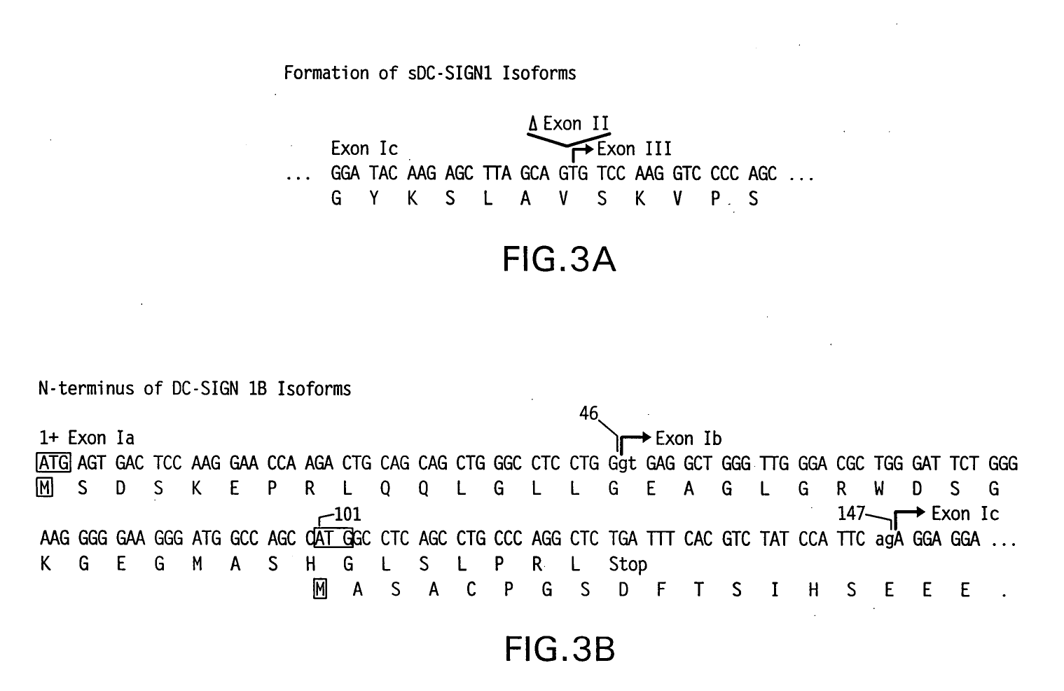

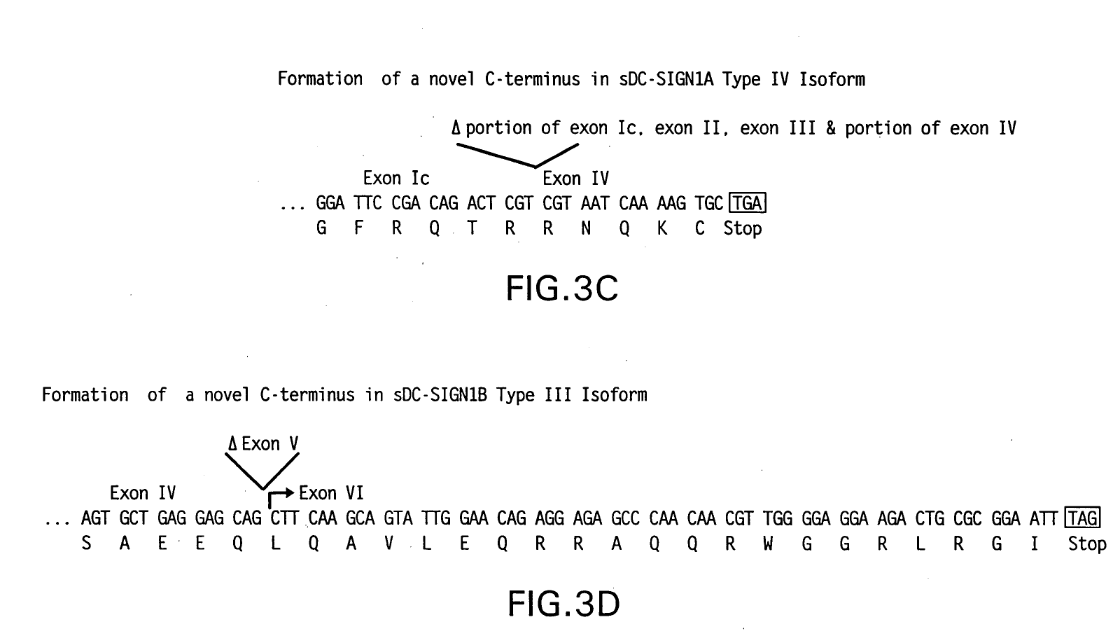

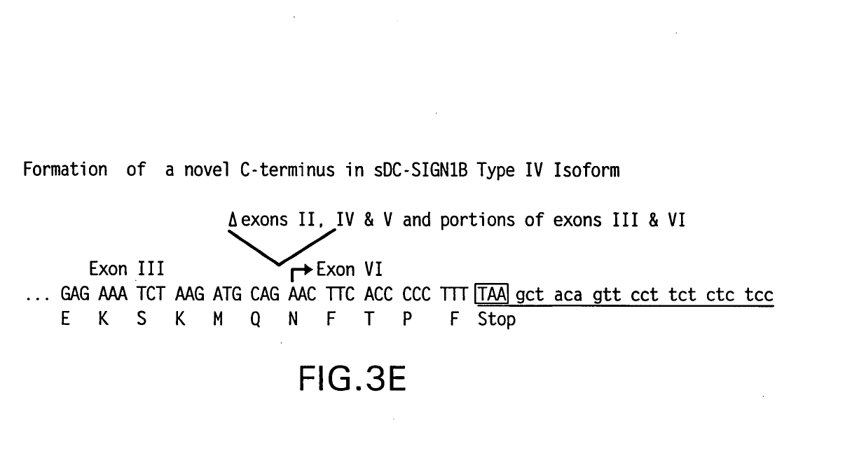

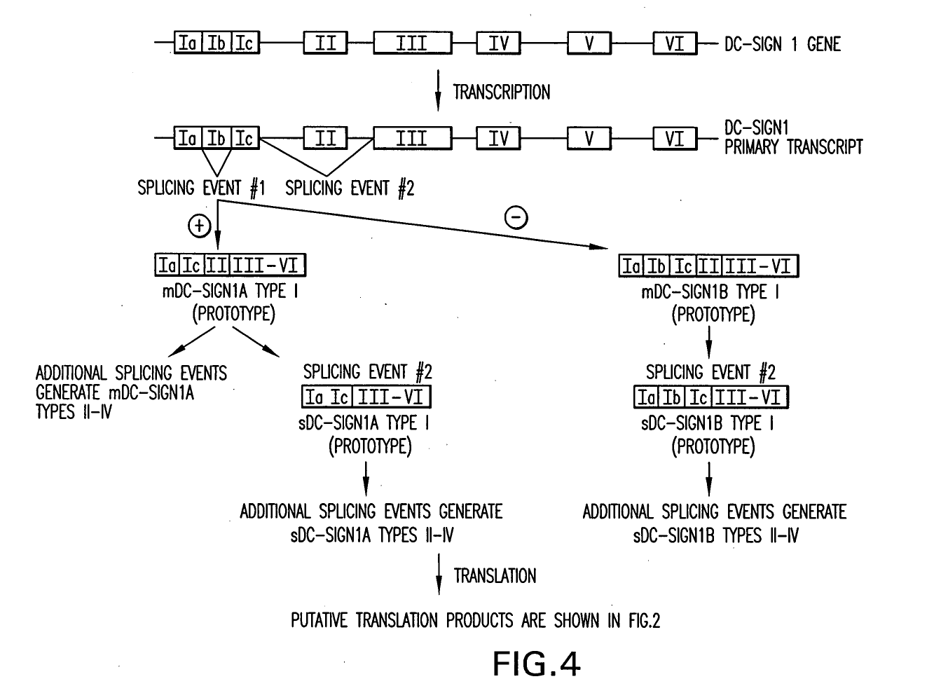

The second group of transcripts was designated as sDC-SIGN1A. sDC-SIGN1A transcripts also had a Met (ATG) translation initiation codon within exon Ia, but the exon predicted to encode the TM domain (exon II) was spliced out, suggesting the synthesis of soluble forms of DC-SIGN1A (FIG. 1a; FIG. 2c; FIG. 3a). The prototypic version of this class of transcripts, designated as sDC-SIGN1A Type I, lacked only the TM-containing exon II, whereas additional splicing events resulted in sDC-SIGN1A Types II-IV (FIG. 1a and FIG. 2c).

The exon Ib-containing DC-SIGN1 cDNAs were collectively designated as DC-SIGN1B transcripts and are predicted to encode the third (mDC-SIGN1B), fourth (sDC-SIGN1B), and fifth (truncated DC-SIGN1B) category of DC-SIGN1 isoforms (FIG. 1b and FIG. 2, d and e). Notably, exons Ia, Ib, and Ic are sequences that are not interrupted by an intron and that collectively comprise exon I. There is a Met (ATG) translation initiation codon within exon Ib, and thus DC-SIGN1 B transcripts can potentially initiate translaton at two sites: +1 or +101 (FIG. 1b, FIG. 2, d and e, and FIG. 3b). The sequence flanking the +101 position has a strong Kozak consensus sequence for initiation of translation (GCCATGG). The deduced amino acid sequence of transcripts that commence translation at the downstream Met codon (i.e. +101) in exon Ib differed from mDC-SIGN1A or sDC-SIGN1A isoforms only in the predicted cytoplasmic domain. These transcripts could be further categorized into those that had (DC-SIGN1B) or lacked (sDC-SIGN1B) the TM-encoding exon II (FIG. 1b, FIG. 2d and FIG. 2e). Notably, prototypic m- or sDC-SIGN1 differed from m- or sDC-SIGN1 B (Type I) by only 14 amino acids in the predicted N terminus encoded by exon Ib (FIG. 2). Finally, usage of the Met codon in exon Ia in DC-SIGN1B transcripts predicted the production of a truncated protein of 41 amino acids (nucleotides +1 to +123; FIGS. 1b, 2, d and e, and 3b), and these isoforms were designated as truncated DC-SIGN1B isooforms (tDC-SIGN1B). To minimize the possibility that the exon Ib-containing DC-SIGN1 transcripts (i.e. DC-SIGN1B mRNAs) reflected PCR amplification of pre-mRNA contaminating the mRNA preparations, the inventors confirmed the presence of these transcripts in poly(A)+ RNA (see below, and data not shown).

Splicing events generated sDC-SIGN1-A or -B transcripts that are predicted to encode novel C termini (FIGS. 1, a and b, 2, c and e, and 3, c-e). In some instances, the splice junctions for the DC-SIGN1 mRNAs did not obey the consensus rules for 5′-intron/exon boundaries (FIG. 1C). Based on the splicing events in exons III-VI, these exons could be further subdivided (e.g. exon IIIa, IIIb, etc.). However, the inventors refrained from doing so, recognizing that based on mRNA expressio analyses there are probably additional splice variants that have not been discovered as of yet (see below). The model shown in FIG. 4 summarizes the predicted DC-SIGN1 gene structure, the primary transcript, mature mDC-SIGN1 (A or B) and sDC-SIGN1 (A or B) mRNAs, and a schema of the potential processing events underlying the formation of the mature messages. Collectively, the findings illustrated in FIGS. 1-4 demonstrate that the DC-SIGN1 gene is subject to highly complex alternative splicing events, generating a wide array of transcripts that are predicted to encode for an extensive repertoire of membrane-associated as well as soluble DC-SIGN1 isoforms with variable intro- and/or extra-cellular regions.

### C. Example 3

### DC-SIGN2

DC-SIGN2, a Gene with Structural Homology to DC-SIGN1 That is Also Subject to Alternative Splicing—By searching the GenBankTM data bases, the inventors found a cDNA (Yokoyama-Kobayashi et al., 1999) and two ESTs (Image Clones 146996 and 240697) that had high overall sequence homology with the DC-SIGN1 transcripts that the inventors had identified. The cDNA and ESTs differed from each other by the presence or absence of additional stretches of sequences. To determine whether the cDNA and ESTs represented allelic versions of the DC-SIGN1 gene or products of a novel gene, RT-PCR was performed on human placenta mRNA using primers specific to those found in the cDNA and ESTs. Sequence analyses of the PCR products revealed additional novel cDNAs with sequences identical to the previously described cDNA/ESTs but distinct from DC-SIGN1 (A or B) transcripts, suggesting that they were alternatively spliced products of a distinct gene and not allelic variants of DC-SIGN1 (FIG. 5). The predicted translation products of these transcripts are illustrated in FIG. 6 and are also shown in Supplementary FIG. 3.

Genomic sequences identical to the novel DC-SIGN-like mRNAs that the inventors had discovered as well as the previously identified cDNA (28) and ESTs were found 15.8 kb centromeric to DC-SIGN1 on chromosone 19pl3.3, and these two genes were arranged in a head-to-head manner (FIG. 7). Based on their close sequence homology, their colocalization on chromosome 19p13.3, and their order of discovery, the inventors designated the previously described DC-SIGN as DC-SIGN1 and this related gene that the inventors had identified as DC-SIGN2. The coding region of the prototypic full-length DC-SIGN1 and DC-SIGN2 shared 84 and ˜80% identity at the nucleotide and protein levels, respectively. Comparison of the DC-SIGN2 mRNA and gene sequences revealed that the coding region of DC-SIGN2 was encoded by seven exons (FIGS. 5a and 6a).

Similar to the alternative splicing events observed in DC-SIGN1, DC-SIGN2 transcripts in which the exon predicted to encode the TM domain (exon III) was spliced in or out were found and were designated mDC-SIGN2 or sDC-SIGN2 isoforms, respectively (FIGS. 5a and 6, a-c). Additional alternative splicing events generated mDC-SIGN2 or sDC-SIGN2 transcripts, which are predicted to encode isoforms with varied extracellular domains (FIG. 5, c and d and 6, b and c). Notably, of the >30 DC-SIGN2 transcripts that the inventors cloned and sequenced from the placenta of a normal donor, 21 cDNAs were found that contained sequences corresponding to intron IV, and in this particular placenta sample, the inventors were unable to identify a prototypic mDC-SIGN2 transcript (FIG. 5). These findings provided the first due that there might be significant inter-individual variability in the repertoire of DC-SIGN2 transcripts expressed in term placenta. The discovery of DC-SIGN2 transcripts with distinct splicing patterns that contained intron IV and/or lacked exon VI from multiple sources (FIG. 6; e.g. ESTs and this study) indicated that the splicing patterns that the inventors found were not aberrant or random events but rather may represent fairly common processing events.

A unique differential splicing event was observed that distinguished DC-SIGN2 mRNAs that contained (mDC-SIGN2) or lacked (sDC-SIGN2) the TM-encoding exon III. Among the DC-SIGN2 cDNAs that the inventors cloned and sequenced, all sDC-SIGN2 transcripts contained sequences corresponding to exon Iva, but none of the transcripts that had the TM-encoding exon III, i.e. mDC-SIGN2 transcripts contained exon Iva sequence (FIGS. 5, a and e and 6c). Exon Iva is predicted to encode a short hydrophobic streth of amino acids (FIG. 5E and Supplementary FIG. 3). It should be noted that intron I of the DC-SIGN2 gene corresponds to exon Ib of the DC-SIGN1 gene. Similar to the scenario observed in DC-SIGN1B, the use of an alternative translational start site at position 111 of intron I in DC-SIGN2 is predicted to encode isoforms with a novel intracellular domain. However, DC-SIGN2 transcripts that contained intron I sequences were not found in the cDNA clones that the inventors have sequenced thus far.

Although these findings provided evidence for extensive alternative splicing events within the region preceding the lectin-binding domain (i.e. region that encodes the repeats) of DC-SIGN2, it was conceivable that in some individuals the variation in the number of repeats could be because of allelic variation. For example, it was conceivable that one allele could encode for eight repeats whereas the other allele could encode for seven repeats. To determine this, the inventors amplified the genomic DNA that spanned the region between exon III and intron IV from normal donors. The inventors found that in some instances, one allele encoded seven repeats whereas the other allele encoded eight repeats. These findings suggested that in addition to alternative splicing, a variation in the number of repeats encoded in the DC-SIGN2 gene could be another source for variability in generating the DC-SIGN2 mRNA repertoire. Additional studies are under way to characterize the nature and frequency of this genetic polymorphism (i.e. variability in number of repeats) in different ethnic populations. Studies are also underway to determine whether there is variability in the number of repeats in the DC-SIGN1 gene.

Addtional inspection of the genomic contig from chromosome 19p13.3, demonstrated that the gene for the low affinity immunoglobulin Fc receptor (CD23), another Type II lectin (Delespesse et al., 1991; Suter et al. 1987), was situated ˜43.3 kb telomeric to DC-SIGN1 (FIG. 7). Thus, DC-SIGN1 (CD209), DC-SIGN2 (CD209L), and CD23 form a cluster of highly related genes, suggesting that they may have arisen by gene duplication of an ancestral gene, and notabl alternative splicing events in all three genes lead to the generation of multiple transcripts (FIGS. 1-7) (Yokoyama-Kobayashi et al., 1999; Yokota et al., 1988; Yoshikawa et al., 1999).

### D. Example 4

### Expression of DC-SIGN1 is Not Restricted to DCs

Expression of DC-SIGN1 Is Not Restricted to DCs—Given the aforemenoned findings, the inventors asked whether the DC-SIGN1 transcripts were expressed in a complementary manner. That is, does a given cell type express only one DC-SIGN1 transcript, similar to the exclusive expression of odorant receptors in olfactory neurons (Chess, 1994), or are different DC-SIGN1 variants expressed in a combinatorial manner? In the first scenario, a given cell type could potentially be classified into one of five groups depending on which DC-SIGN1 tanscript it expressed. In the second scenario, distinct transcripts could be co-expressed in variable patterns to confer specific properties onto the expressing cells, with the variability being dependent on the ratio of expression of the different DC-SIGN1 mRNAs. An additional level of complexity could be that the expression patterns varied depending on the activation state and/or maturation stage of the cell.

To address the aforementioned question, a RT-PCR-based strategy that included Southern blot hybridization was used to determine the expression of DC-SIGN1 mRNAs in primary human cells and human cell lines. To perform semiquantitative RT-PCR, in initial experiments the inventors determined the number of PCR cycles wherein the hybridizing signal for DC-SIGN1 cDNAs were in the linear range (30 cycles), and PCR was performed using equal (1 Âμg) amounts of mRNA from each cell/tissue type.

To increase the specifity and to estimate the relative amounts of DC-SIGN1 mRNAs that had or lacked the TM-encoding exon II, five procedures were adopted. First, PCR was performed using unlabeled oligonucleotides specific for DC-SIGN1A or DC-SIGN1B (Table I), and the PCR products containing the DC-SIGN1 cDNAs were transferred to a membrane, and hybridized using DC-SIGN1 (A or B)-specific internal 32P-labeled oligomers. This srategy assured that the hybridizing signal contained the DC-SIGN-specific sequence and not nonspecific amplification.

Second, because DC-SIGN1 and DC-SIGN2 transcripts shared high sequence homology, the specificity of the nested radiolabeled DC-SIGN1 probes and washing conditions were optimized in control experiments using cloned DC-SIGN1 and DC-SIGN2 cDNAs. Four nested radiolabeled oligomers were used in these hybridization studies (Table I). (i) The exon VI oligomer was designed to hybridize DC-SIGN1A and DC-SIGN1B transcripts regardless of whether they contained or lacked the TM-encoding exon II. This oligomer hybridized specifically to mDC-SIGN1, and a very faint cross-hybridizing signal was detected in mDC-SIGN2 cDNAs. (ii) The exon II oligomer was designed to hybridize transcripts that contained the TM-encoding exon II, i.e. mDC-SIGN1 (A or 1B) mRNAs. This probe specifically hybridized mDC-SIGN1 but not sDC-SIGN1, mDC-SIGN2, or sDC-SIGN2 cDNAs (data not shown). (iii) The exon Ic-exon III oligomer is specific for sDC-SIGN1 (A or B) DNA, i.e. transcripts that lacked exon II. Notably, this probe did not hybridize to DC-SIGN1 or -2 transcripts that contained the exon II-encoding TM domain or to sDC-SIGN2 DNA (data not shown). (iv) The exon Ib oligomer was designed from a region that is not found in DC-SIGN1A transcripts, and in hybridization studies it was specific to m- or sDC-SIGN1B cDNAs (data not shown). Third, to confirm that the DC-SIGN1 PCR primers used to generate the cDNAs were specific, the Southern blots were stripped of radioactivity and reprobed with primers specific to DC-SIGN2. On rehybridization, DC-SIGN2 cross-hybridizing signals were not detected.

Fourth, because of the very faint cross-hybridizaton signals observed with the exon VI probe, the inventors designed oligomers specific to DC-SIGN1 exon Ic (oligomer 1-5; Table I) and DC-SIGN2 exon II (oligomer 2-6; Table I). A set of Southern blots were hybidized with either a radiolabeled DC-SGN1 exon Ic or DC-SIGN2 exon II probe. Hybridizing signals obtained with the DC-SIGN1 exon Ic probe were identical to those observed previously with the DC-SIGN1 exon VI probe. In contrast, a hybridizing signal was not detected with the DC-SIGN2 exon II probe, indicating that the mRNA expression patterns observed using the strategy outlined was specific for DC-SIGN1. As a final step to increase specificity and validate the expression pattern of DC-SIGN1 and DC-SIGN2 transcripts, cDNAs were synthesized from multiple different normal donors and cell lines.

An example from four separate experiments demonstrating the cell and tissue expression of DC-SIGN1 transcripts was observed. The inventors first focused on the expression of DC-SIGN1 mRNA in CD34+ PBHP cells cytokine-differentiated toward the DC lineage. M- and sDC-SIGN1 (A or B) cDNAs were abundantly expressed in mature DCs, i.e. CD34+ PBHPs cytokine-differentiated for 15 days but not at earlier time points. In addition to the prominent hybridizing signals of ˜1-1.3 kb in length, several hybridizing bands that were <1 kb in length were also detected (see below and data not shown). Notably, the hybridizing signal in CD34+ PBHPs differentiated with IL-4 was stronger than that observed in DCs cultivated without IL-4 (day 15 Â±IL-4), suggesting that the expression level of DC-SIGN1 mRNA may be dependent on the maturational/activation state of DCs. On longer exposures, fain hybriding signals were evident at day 8 and 12 cytokine-differentiated CD34+ PBHPs, suggesting that the expression of DC-SIGN1 in immature DCs was significantly lower than that in mature DCs derived from CD34+ PBHPs.

In addition to DCs, m- and sDC-SIGN1 (A or B) transcripts were expressed in other antigen-presenting cells such as highly purified resting CD14+ monocytes (data not shown) as well as THP-1 and U937 cells, two monocytic cell lines (data not shown). Expression of DC-SIGN1 transcipts was confirmed in two independent sources of THP-1 cells (ATCC and National Institutes of Health AIDS repository; data not shown). Because it was difficult to control for differences in the labeling and hybridizing efficiencies of the different probes required to differentiate between the exon II-containing or -lacking DC-SIGN1 transcripts, it was not possible to assess in a quantitative manner their relative abundance in DCs or THP-1 cells. Nevertheless, the findings indicated that both m- and sDC-SIGN1 (A or B) transcripts are abundanty expressed in DCs and THP-1 cells.

Weak expression of DC-SIGN1 mRNA was detected in resting PBMCs obtained from eight normal donors (data not shown). In contrast, abundant expression for m- and sDC-SIGN1 (A or B) transcripts was detected in all eight PBMC samples after stimulation with PHA (data not shown) as well as in PBMCs activated with CD3/CD28. DC-SIGN1-specific hybridizing signals were evident in PBMCs activated with PHA for 4 days but not in PBMCs cultured in PHA (days 1-4) plus IL-2 (days 5-12). Notably, there was inter-individual variation in the expression of DC-SIGN1 transcripts in PHA-activated PBMCs.

Because of the interest in the potential role of HIV attachment factors such as DC-SIGN1 in mother-to-child transmission of the virus, the inventors also determined whether DC-SIGN1 is expressed in the placenta. Notably, the inventors detected both inter-individual variation in the levels of DC-SIGN1 expressin as well as heterogeneity in the repertoire of transcripts expressed. The expession of DC-SIGN1 in placenta was confirmed by immunohistochemical staining of term placentae. DC-SIGN1 expression colocalized with that of PECAM, an endothelial cell marker, as well with CCR5. Double immunostaining indicates that DC-SIGN1 is co-expressed along with CCR5 in placental villi, and the distribution pattern of CCR5+DC-SIGN1+ cells is consistent with their expression in villous marophages.

Weak DC-SIGN1-specific hybriding signals of ˜1.2 kb in length were also observed in MG63 (osteoblast) cells, HSB-2 (T cells), and MC116 cells, a B-cell line (data not shown). DC-SIGN1 expression was observed in the T cell line, HUT78; however, only an ˜300 and ˜600 bp hybridizing signal was detected in this cell type (data not shown). The presence or absence of hybridizing signals of varying sizes in T cells might reflect differences in the activation states of these cell lines. A ladder of hybridizing bands was also observed in HL-60 cells, a granulocylic cell line (data not shown).

The strongest hybridizing signals for DC-SIGN1 in mature DCs, PBMCs, placenta, and THP-1 cells were in hte 1,000-1,300-bp range, which was concordant with the large number of transcripts identified in this size range by direct cDNA sequencing (FIGS. 1 and 2). However, the strong intensity of the hybridizing signals at ˜1-1.3 kb masked the ladder of hybridizing bands that was evident on shorter exposures (data not shown) (Ex VI probe). Furthermore, the inventors found it difficult to resolve this ladder of hybridizing bands using horizontal gel electrophoresis. To circumvent this limitation, the DC-SIGN1 sense orientation oligomer used in the aforementioned experiments was readiolabeled and used in PCR, and the amplicons were resolved on a polyacrylamide gel. The findings of these experiments revealed a ladder of PCR amplicons in all cell types having size ranges concordant with the lengths of the DC-SIGN1A or DC-SIGN1B cDNAs that the inventors had identified by direct sequencing (data not shown). This ladder of PCR amplicons is consistent with the notion that DC-SIGN1 undergoes extensive splicing events to generate a large repertoire of transcripts of varying lengths. The lengths of the transcripts in the 1-1.3-kb size range may appear deceptively similar, and direct sequencing may be necessary to distinguish their unique sequence characteristics.

The inventors next determined whether the transcripts predicted to encode membrane-associated and soluble DC-SIGN1 isoforms are translated in vitro. The in vitro translated products of the predicted sizes (epitope tag plus coding region) for both DC-SIGN1A and DC-SIGN1B products confirmed the integrity of the coding regions of the transcripts shown in FIGS. 1 and 2.

### E. Example 5

### Expression of DC-SIGN1 Transcripts That Lack or Contain the Transmembrane™ Encoding Exon in DCs and THP-1 Cells

Total RNA (10 g) was isolated from DCs derived from cytokine-differentiated CD34+ PBHPs, PBMCs, placenta, THP-1 cell line or other cell lines (data not shown) was reverse transcribed with oligo(dT) primers. The resulting cDNA was PCR amplified using DC-SIGN1A SER ID NO: (primers 1-1 and 1-2) or DC-SIGN1B SEQ ID NO: (primers 1-3 and 1-2) specific primers. The PCR amplicons were fractionated by agarose gel (1.5%-) electrophoresis, transferred to Nylon membrane, and hybridized with the indicated radiolabeled probes. The blots were washed and then exposed for 15 h.

Specificity of the radiolabeled oligomers used. Nylon membranes spotted with the mDC-SIGN1 and mDC-sign2 DNA were hybridized with a radiolabeled probe. Ex VI probe hybridies all DC-SIGN1 (A or B) transcripts; Ex II probe hybridizes all DC-SIGN1 (A or B) transcripts that contain the TM-encoding exon II; Ex Ic-Ex III probe hybridizes DC-SIGN1 (A or B) transcripts that lack the TM-encoding exon II.

The inventors observed DC-SIGN1 (A or B) expression in CD34+ PBHP differentiating DCs cultured in the presence or absence of IL-4. Activation-induced differences in the levels of DC-SIGN1 expression (compare hybridizing signal in DCs±IL-4). The probes used were Ex VI, Ex II and Ex Ic-Ex III.

CDNAs amplified using DC-SIGN1B-specific primers from DCs derived from cytokine-differentiated CD34+ PBHPs or THP-1 cells were fractionated by gel electrophoresis and Southern blot hybridized with the radiolabeled Ex VI oligomer or a radiolabeled oligomer that is specific to DC-SIGN1B.

The inventors also observed DC-SIGN1 (A or B) expression in THP-1 cells obtained from ATCC. The probes used were Ex VI, Ex II and Ex Ic-Ex III.

### F. Example 6

### Differential Expression Levels of Transcripts Predicted to Encode Membrane-Bound and Soluble DC-SIGN1 Isoforms in Resting Versus Activated PBMCs of Normal Donors

The overall experimental strategy for the findings is identical to that used in Example 5. Five donors were used. The Expression of all DC-SIGN1 transcripts (Ex VI probe) or trnascripts tht contain (Ex II probe) or lack (Ex Ic-ExIII probe) the TM-encoding exon II in resting and PHA-activated (for 4 days) PBMCs were derived from normal donors. THere was a variability in the mRNA expression in one of the donors when compared to the other four donors.

A photomicrograph of ethidium bromide-stained agarose gel showing DC-SIGN1 amplicons was obtained by the inventors.

The inventors observed mRNA expression of DC-SIGN1B in PHA-activated PBMCs. Oligo(dT)-primed PBMC cDNAs were PCR amplified with DC-SIGN1B-specific primers, and the resulting PCR amplicons were fractionated by agarose gel electrophoresis and then Southern blot hybridized with an oligomer specific to DC-SIGN1b. The inventors also observed DC-SIGN1 mRNA expression in PBMCs activated for 4 days with PHA, or PHA plus IL-2, or CD3 plus CD28.

The inventors further observed expression of DC-SIGN1 transcripts in placenta or three normal donors.

### G. Example 7

### Expression of DC-SIGN1 Protein on Vascular Endothelium and Macrophages of Placenta

The inventors observed expression of DC-SIGN1 protein on vascular endothelium and macrophages of placenta colocalized with that of PECAM, and endothelial cell marker. In a negative control assay, it was observed that immunohistochemical staining was blocked by DC-SIGN1 and PECAM specific peptides.

In another negative control assay, the immunohistochemical staining was blocked by DC-SIGN1 and CCR5 peptides.

### H. Example 8

### Extensive Repertoire of DC-SIGN1 mRNA Transcripts in DCs, THP-1 Cells, and PBMCs, and In Vitro Translation of DC-SIGN1 cDNAs

Oligo(dT)-primed cDNAs were PCR amplified with DC-SIGN1-specific primers. The sense-orientation primer was ³²P-endlabeled, and the resulting PCR amplicons were fractionated on a 4% polyacrylamide gel. PCR-amplified products of 11 cDNAs shown in FIG. 1 (FIB. 1B through FIG. 1E: m- and sDC-SIGNA Types I-IV; mDC-SIGN1B Type I; sDC-SIGN Types I and II). The in vitro translation products of the mDC-SIGN1A transcripts were observed as follows: Type I; 48.7 kDa; Type IV; 22.6 kDa; Type II; 48.1 kDa; Type III; 38.7 kDa.

The in vitro translation products of sDC-SIGN1A transcripts are were observed as follows: Type III; 36.2 kDa; Type I; 48.1 kDa and Type II; 43.9 kDa. Although the predicted size of the prototypic DC-SIGN1A is ˜44 kDa, the in vitro translated product is ˜48 kDa.

### I. Example 9

### mRNA Expression of DC-SIGN2 and Interindividual Variation in the Expression of DC-SIGN1 and DC-SIGN2 Transcripts in Placenta of Normal Donors

Expression of DC-SIGN2 was observed in the placenta. Oligo(dT)-primed placenta cDNAs were PCR amplified with DC-SIGN2-specific primers (primers 1-1 and 2-5). The sense-orientation primer was ³²P-end labeled, and the resulting PCR amplicons were franctionated on a 3% polyacrylamide gel.

The inventors observed interindividual variation in the expression of DC-SIGN2 transcripts in placenta. DC-SIGN2 specific primers were used to PCR amplify oligo(dT)-primed cDNAs from 10 different individuals and the products were analyzed as described above. The inventors also observed mRNA expression of DC-SIGN2 in DCs derived from cytokine-differentiated CD34+ PBHPs.

### J. Example 10

### Production of Encombinant Soluble DC-SIGN1 Isoforms and Immunoblotting of the Recombinant DC-SIGN Proteins

His recombinant mDC-SIGN1 full length protein was produced by cloning the cDNA sequence into pcDNA/HisMax TOPO Vector (Invitrogen) and expressed in the Hela cell line. Membrane fraction of the cell pellet was stained with a polyclonal and a monoclonal DC-SIGN1-specific antibody. The tags and cleavage sites add an additional 3.9 kD to the final size of the protein.

SDC-SIGN1 variants were cloned into the pMIB/V5-HisA Vector (Invitrogen) and transfected with Fugene 6 (Roche Biochemicals) into Sf9 Insect cells. The construct is in frame with an artificial secretion signal and a nine amino acid HA tag were added at the amino-terminus. Cells were starved for 24 hours and supernatant were collected and sDC-SIGN1 was purified by using the HA affinity column according with the manufacturer's protocol (Roche Biochemicals). SDC-SIGN1A type I isoform was obtained by using DC-SIGN1 polyclonal antibody and immunoblotted with a HA monoclonal antibody. This isoform only lacks the transmembrane domain (FIG. 1c)

sDC-SIGN1A type III (SEQ ID NO: 6) isoform was also obtained. This isoform lacks the transmembrane domain and exon Ic and some repeats. Purified sDC-SIGN1A type I protein, i.e. the isoform that only lacks the transmembrane domain was also obtained. The purified protein was immunoblotted with the polyclonal DC-SIGN1 antibody.

Purified sDC-SIGN1A type I protein, i.e. the isoform that only lacks the transmembrane domain immunoblotted with the HA antibody was also obtained. Silver staining of the purified sDC-SIGN1A type I protein demonstrates the relative purity of the protein.

### K. Example 11

### Expression Pattern of DC-SIGN2 Transcripts

To determine the expression of DC-SIGN2 transcripts, a strategy similar to that used to examine the expression of DC-SIGN1 was adopted (data not shown). In initial experiments, the inventors observed that akin to DC-SIGN1, DC-SIGN2 transcripts were expressed in the placenta, and concordant with the isolation of cDNAs of varied lengths from this tissue, a ladder of amplicons were observed in some placental samples. However, in these initial experiments, the inventors found that there was extensive inter-individual heterogeneity in not only the expression levels but also the repertoire of transcripts expressed. For example, the inventors found that placenta from donor 3 lacked a transcript in the size range for the prototypic mDC-SIGN2 mRNA. This finding was notable because it may explain, in part, why the inventors were unable to directly clone mDC-SIGN2 Type I transcripts from mRNA derived from this placenta sample. In agreement with the cDNA cloning studies, all four placenta samples had transcripts in the size ranges that were consistent for expression of intron IV-containing mRNAs, suggesting that these transcripts may comprise a major proportion of the DC-SIGN2 mRNA repertoire. As indicated earlier, variability in the length of the DC-SIGN2 transcrips may be accounted for, in part, by the variation in the number of repeats present on a given allele.

To extend and confirm these findings, the inventors determined the expression of DC-SIGN2 transcripts in 10 additional term placentae from normal donors. Consistent with the initial studies, the inventors found that there was striking heterogeneity in both the levels of expression of DC-SIGN2 transcripts as well as in the repertoire of transcripts expressed. Notably, despite equal expression for actin in all placental samples, the inventors were unable to detect transcripts for DC-SIGN2 in 4 of the 10 placental samples, and only 2 of 10 placenta mRNA samples (samples 11 and 12) had tanscripts with lengths corresponding to the prototypic mDC-SIGN2 mRNA.

DC-SIGN2 amplicons were also found in mature DCs (day 15 cytokine differentiated CD34+ PBHPs). However, using a RT-PCR Southern blot hybidization, the expression of DC-SIGN2 in CD34+ PBHP-derived mature DCs was found to be significantly lower than thad of DC-SIGN1 (data not shown). Using the Southern blot hybridization strategy, weak expression for DC-SIGN2 was also detected in THP-1 monocytic cells, whereas expression was not detected in CaCo2 (colorectal adenocarcinoma), RD (rhabdomyosarcoma), HUT 78 (T cell), MC116 (B cell) cells, or resting or activated PBMCs (data not shown).

The inventors also observed DC-SIGN2 expression in ten different donors RNA from placenta fron 10 different healthy people was isolated, quantified and used for cDNA synthesis. PCR was conducted in order to amplify the whole coding region and a series of Nested PCRs using primers lying in areas known for variations was conducted. There are differences in the set of variants expressed among normal people.

### L. Example 12

### DC-SIGN2 Expression in Choriocarcinoma Cell Lines

The inventors observed DC-SIGN2 expression in choriocarcinoma cell lines. A panel of cell lines were tested for DC-SIGN2 expression by RT-PCR and Nested PCR. The cell lines express different versions of transcripts (including soluble and membrane-isoforms). Choriocarcinoma arise from trophoblast, which are the cells that enter in direct contact with the maternal blood flow. The expression of DC-SIGN in these cells may influence vertical HIV-1 transmission. The role of the different isoforms and its variation could explain the differences in probability of infection.

### M. Example 13

### DC-SIGN2 Expression in Trophoblast Cells

Trophoblasts cells purified from normal placenta were isolated by Ficoll gradient and RNA was extracted. RT-PCR was done using different sets of primers. The trophoblast cells express DC-SIGN2 and there were differences in its quality and quantity of mRNA variants.

### N. Example 14

### mRNA Expression of DC-SIGN1A, DC-SIGN1B and DC-SIGN2 on Placenta from Three Donors

The inventors observed mRNA expression of DC-SIGN1A, DC-SIGN1B and DC-SIGN2 on placentas from three donors. Five fractions were colleced from different sites within the same placenta. RNA isolation and RT-PCR was conducted for each sample. The inventors found differences in expression, but also in the repertoire of transcripts been produced even within different fractions of the same placenta. The whole repertoire of transcripts may account for the net effect of the DC-SIGN genes in HIV-1 vertical transmission as well as other pathogens that use DC-SIGN as a receptor in transuterine infection. Also the immune response in placenta mediated by DC-SIGN genes may be impacted.

### O. Example 15

### DC-SIGN3 mRNA Expression in Different Cell Lines

The inventors observed DC-SIGN3 mRNA expression in different cell lines: (1) DN145; (2) K562 (Bone marrow Leukemia); (3) MiaPACA2 (Pancreas Carcinoma); (4) WKPanc-1(Pancreas Carcinoma); (5) Hut78 (Cutaneous T Lymphocyte; Lymphoma); (6) THP-1 (Monocyte; Acute Monocytic Leukemia); (7) LnCap1 (Prostate; metastatic site: Left subclavicular lymph node carcinoma); (8) HBL100 (Breast; Epithelial Carcinoma), (9) HeLa (Cervix; Epithelial; Adenocarcinoma); (10) MCF7 (Mammary Gland; Breast; metastatic site: Pleural effusion Adenocarcinoma); (11) MDA435 (Mammary gland, breast, duct, metastatic site: Pleural effusion ductual carcinoma); (12) CAPAN1 (Pancreas; Metastatic site: Liver adenocarcinoma); (13) 293T (Human Embrionic Kidney); (14) PC3 (Prostate; metastatic site: bone Adenocarcinoma); (15) Panc1 (Pancreas, duct, epitheloid carcinoma); (16) CAPAN2 (Pancreas; adenocarcinoma), and (17) Hs578T (Mammary gland, Breast). DC-SIGN3 expresson is wide, however the amount of mRNA transcripts is low since a Nested PCR is required to unravel the bands.

### P. Example 16

### Recombinant Protein Expression in Insect Cells

The inventors observed recombinant protein expression in Insect Cells. DC-SIGN3 cDNA was cloned in pMIB/V5-HisA vector (Invitrogen) by using Not I and HindIII restriction sites. Sequence integrity and open reading frame was confirmed by sequencing. Sf9 cells were transfected with the DC-SIGN3 construct by using Fugene 6 reagent (Roche Biochemicals). As a control the inventors cloned the insert in the opposite orientation in the same vector and transfected as a mock transfection. After transient transfection (48 hours) cells were starved for 24 hours with serum free media and collected supernatant was immunoblotted with a 6His Tag antibody (R&D). A system for protein production was set up and purified protein.

Further assays will be conducted by the inventors to study the biological properties of this molecule, including, but not restricted to its ability to bind HIV-1 envelope glycoprotein gp120.

### Q. Example 17

### Qualitative mRNA Analysis of DC-SIGN1 and DC-SIGN2 and Their Isoforms

To obtain an initial picture of the cell types in which DC-SIGN1 and DC-SIGN2 is expressed, the iventors will profile gene expression using the “Human Multiple Tissue Expression (MTE) Array (Contech).” In a single hybridization experiment using gene-specific primers, the inventors will obtain a broad assessment of expression in 76 tissue-specific poly A+ RNAs. The array is normalized to eight different housekeeping genes.

A TapMan (Real-Time) PCR and/or molecular beacon assay (Johnson et al., 2000; Kafert et al., 1999; Blaschke et al., 2000) using gene-specific primers will be used to precisely quantitate the mRNA distribution of DC-SIGN1 and DC-SIGN2 isoforms.

### R. Example 18

### Generation and Characterization of DC-SIGN Monoclonal Antibodies

The inventors have been using well-established protocols for immunizing, selecting, subcloning and characterization of monoclonal antibodies (Wu et al., 1996; Bleul et al., 1997). These protocols have been used successfully for generating monoclonal antibodies to cell surface molecules, including CCR5 of African green monkeys (AGM). In these experiments, CCR5-AGM was successfully expressed at high levels in both human cells and mouse cells (LA9), and the latter CCR5-expressing cells were used to generate monoclonals (unpublished data).

The full-length isoforms of DC-SIGN1 and DC-SIGN2 will be cloned into the pcDNA4/HisMax (Invitrogen) vector, and tansfected into LA9 cells. Expression of DC-SIGN in LA9 transfectants will be determined by examining for expression of the HIS-tag in permeabilized cells (the HIS-tag is at the amino terminus (cytoplasmic)). Mice will be immunized by intraperitoneal inoculation (IP) on day 0, 14, and 21 with murine DC-SIGN1-transfected LA9 cells (100 μl containing 5×10⁵ cells in PBS). Splenic cell suspensions will be fused with a mouse myeloma cell line (SP-2) and seeded into 96 well round bottom wells as previously described. The clones will be visualized and culture supernatants tested for antibody production. Screening of hybridoma supernatants will be performed by examining cell surface staining of LA9 DC-SIGN cells by FACS. The inventors typically screen between 100-200 hybridoma supernatants a day (total of 400-600 hybridomas) by this procedure. Positive wells will be assessed for specifity by differential binding compared with non-transfected LA9 cells. DC-SIGN positive hybridomas will be subcloned by limiting dilution and monoclonal antibodies re-tested, purified, and the antibody concentration, isotype determined, and conjugated with PE. A second group of mice will be immunized with DC-SIGN2 to generate monoclonals reactive with DC-SIGN2, but not DC-SIGN1.

### S. Example 19

### Determination of DC-SIGN Density on DC Cells and Other Expressing Cell Types Using Monoclonal Abs

Geijtenbeek has reported that DC-SIGN is expressed at very high levels in DCs (Geijtenbeek et al., 2000; Geijtenbeek et al., 2000a; 2000b). One important consideration is whether the relative levels of DC-SIGN1 expression would influence the amount of HIV-1 captured and hence impact trans-infection of resident T cells. Several methods have been used to examine the number of chemokine receptor molecules present on PBMCs and DCs from humans (Hladik et al., 1999; Lee et al., 1999; Reynes et al., 2000), and these methodologies can be used to assess the density of DC-SIGN1 and is isoforms on DCs and other expressing cell types. While mean fluorescence intensity (MFI) is used generally to assess receptor density on the cell surface, MFI is does not provide an absolute value such as the number of molecules of a given receptor and may not recognize subtle differences in DC-SIGN expression in different cell types. Instead, the inventors will directy quantify the number of DC-SIGN molecules on the suface of the cell with calibrated PE-conjugated bead (QuantiBrite, Becton Dickinson). The DC-SIGN to PE conjugate ratio of 1:1 will allow for meaningful comparisons with the QuantiBrite beads. Direct measurement of DC-SIGN density is established by first constructing a calibration curve of PE-conjugated beads followed directly by acquisition of PE-conjugated anti-DC-SIGN stained cell populations. Calculations are made using QuantiCALC software. The inventors will compare the relative densities of DC-SIGN, CCR5 and CD4 on DCs and other expressing populations and will determine if there is a direct correlation between DC-SIGN density and capture by HIV and trans-infection of activated PBMCs (aim #3).

### T. Example 20

### Viral Glycoprotein Adhesivity/Binding and HIV-1 Trans-Infection Activity of Transmembrane-Containing DC-SIGN1 (mDC-SIGN) and DC-SIGN2 Isoforms

The inventors will (a) clone the full-length mDC-SIGN1a, mDC-SIGN1b, and mDC-SIGN2, and the mDC-SIGN1 isoforms into the pcDNA4/HisMax (Invitrogen) vector. The configuration of this vector is such that it attaches a His-tag at the intracellular, N-terminus of mDC-SIGN1; (b) Generate permanent HEK293T and K562 cell lines expressing the full-length N-terminus HIS-tagged mDC-SIGN1 using methods described previously (Ahuja et al., 1996; Alkhatib et al., 1997). The inventors selected these two cell lines because they do not express CCR5 or CD4, and Geijtenbeek et al. (2000a; 2000b) have used K562 to successfully express DC-SIGN1; (c) Screen cell lines for expression levels using the anti-HIS antibody (Novagen; after permeabilization because HlS-tag is intracellular). Both FACS and immunoblotting with the rabbit antiserum against DC-SIGN1 and DC-SIGN2 will be used for determining expression. Once the monoclonal DC-SIGN1 Abs are available, they will be used for determining expression levels. The high expressors will be selected for analysis; (d) Determine gp120 and ICAM-3 adhesity to the mDC-SIGN1 (full length and alternatively spliced)-expressing cell lines using the binding assays described in preliminary studies. In these assays, FACS is used to measure the percentage of cells that had bound fluorescent beads. Ligand affinity will be determined by titrations of soluble gp120 and measuring the binding to mDC-SIGN1 transfectants. Specifity will be determined by using antibodies to DC-SIGN1. Heterologous competition assays with soluble ICAM-3 will also be conducted. As a control, in each analysis gp120 affinity for the full length DC-SIGN1 isoform will be included. The sensitivity of the gp120 binding to Ca²⁺ will also be determined, and for these assays Ca²⁺ affinity will be determined by measuring the binding of DCs to gp120 beads at different Ca²⁺ concentrations. Specificity will be determined in the presence of DC-SIGN antibodies. The resulting curves for gp120 will be fitted to the equation for second order dependence to Ca²⁺ (Geijtenbeek et al., 2000a; 2000b; Mullin et al., 1997) Fractional binding=[Ca²⁺]²/((K_{Ca})²⁺[Ca²⁺]²); (e) If mDC-SIGN2 and mDC-SIGN1 isoforms demonstrate gp120 adhesivity, their ability to facilitate infection of HIV permissive cells (293T cells expressing CD4/CCR5) in trans will be determined as described below; (f The aforementioned studies will have provided us information regarding the important segments within the extracellular domains that mediate gp120 adhesivity. To extend these studies, a panel of alanine and other relevant point mutants will be generated by site-directed mutageneis to delineate precisely the residues in the ectodomains that mediate mDC-SIGN1 (or mDC-SIGN2) gp120 adhesivity and HIV-trans-fection activity.

### U. Example 21

### Binding Kinetics

Surface plasmon resonance (SPR) studies not only localize the gp120 binding sites on DC-SIGN1, but more importantly, determine the binding kinetics. Binding affinity data will be generated using a BIAcore 3000 biosensor in collaboration with Dr. Eileen Lafer, who has extensive experience using this technology for quantification of protein-protein interactions and is also the head of the Biacore Core facility at UTHSCSA. In a typical SPR experiment, the molecule of interest (e.g. DC-SIGN1 peptides or soluble lectin-binding domains of DC-SIGN1) are immobilized on the sensor surface (chip), and an interacting molecule (in this case gp120) is passed over the surface. Polarized light is directed on the surface, and the instrument's optical detection unit measures the angle of reflection of the light. The change in the angle of the reflected light (the resonance signal) is directly proportional to the mass on the surface. If the molecule in solution does not interact with the surface molecule, there will be no mass change and therefore no SPR signal. However, if the molecules do interact, then the mass on the surface increases and the interaction is observed in real time as a resonance signal. The progress of the reaction as a function of time is monitored in a plot called a “sensor grade”. Information concerning the rates of association and dissociation of the reaction, as well as steady state concentrations can be determined using this technology. If dissociation rates are too slow to measure in real time, the surfaces can be regenerated by treatment with an agent that disrupts the interacton so the surface can be re-utilized to study a new binding event. A wide variety of surfaces (chips) are available based on chemical properties for anchoring proteins/peptides. The most widely used is the CM5 (carboxymethyl dextran) chip that binds to thiols, although the NTA chip containing chelated nickel is useful for binding to HIS-tagged proteins. The inventors will prepare negative control surfaces with proteins which are not expected to interact such as ovalbumin that will be modified to bind to the chosen surface. Alternatively, they will also pass control proteins over our specific surfaces to insure that irrelevant proteins are not retained.

First the optimal density of the attached DC-SIGN1, will be determined. The DC-SIGN1 lectin-binding domain will be expressed with a HIS-tag, for HIS coupling to an NTA chip. The SPR studies will be used to demonstrate that the recombinant portion of the DC-SIGN1 lectin-binding domain as defined in our functional gp120 assays binds to gp120 in solution. A dose response of the gp120 will be performed to determine optimal binding. This will allow us to establish a binding and regeneration protocol. Then the binding site will be further mapped utilizing additional recombinant soluble portions of the DC-SIGN1 lectin-binding domain, such as exon V-VI, exon VI-VII, exon V, exon VI or exon VII. Fine site mapping will be accomplished by alanine scanning mutagenesis of the appropriate recombinant fragment. Once the binding site has been identified it will be verified using shorter synthetic peptide versions of the defined protein segment utilizing the internal cysteines for coupling to a CM5 matrix through the free thiols. This will be further confirmation and validation of the HIS-tag studies.

Alternatively, the inventors will immobilize gp120 since immobilization and regeneration protocols have already been established for this glycoprotein. Next, kinetic analyses will be performed by utilizing low-density surfaces and high flow rates. Data from these analyses will be evaluated using the global analysis software, BIAevaluation 3.0. Fits will be performed using biomolecular interaction models. These should give accurate analyses of the kinetic characteristics and permit a comparison of the kinetics and affinities for the different interactions measured.

### V. Example 22

### Differences in Binding Affinities of DC-SIGN1 Isoforms Predict HIV-1 Capture and Trans-Infection of Susceptible T Cells

K562 cells expressing DC-SIGN1 and 2 isoforms (5×10⁵) will be incubated with R5- and X4-tropic HIV-1 at a MOI of 0.05; the transfected cells are washed and then incubated with an equal number of CXCR4+/CD4+ or CCR5+/CD4+ transfected 293T cells. The inventors will use an HIV-1 pseudotype luciferase reporter gene assay that is quantitative and highly sensitive in detecting single cycle infection. Dr. Natn Landau (Scripps Research Institute) has kindly provided us with the following constructs; pNL-Lu-E-R+, and pSV-A-MLV-Env, pCMV-VSV-G (to control for cell viability). PNL-Luc-E-R+ is a vector that encodes the HIV-1 genome with the luciferase gene in plae of env (Connor et al., 1995). To derive virus stocks, the inventors have co-transfected 293T cells with pNL-Luc-E-R+, and the following env constructs; HIV-1 (R5) Ba-L, ADA, JRFL, (X4) JC2 and 1A11 (SIVmac,R5). The amount of luciferase activity from each lysate will be determined with a Lumat LB 9501 luminometer and reported as light units or % of VSV-G envelope activity and should correlate with number of cells infected by co-culture with DC-SIGN transfected cells. This assay has also been used previously for determinatons of HIV-1 capture by DC-SIGN (Geijtenbeek et al., 2000a; 2000b).

The luciferase pseudotype assay is highly sensitve for detecting viral entry, however it is limited in that viral replication cannot proceed. To determine if differences in HIV-1 capture (gp120 binding) leads to differences in productive infection, both R5-tropi and X4-tropic infectious virus generated on appropriate cell types (BaL is grown on macrophage cultures; X4-tropic viruses are grown on T cell lines) will be examined as above except that the co-cultures will be followed for 21 days and assessed for virus production by antigen capture (p24 ng/ml). All virus stocks will be normalized for TCID50 on activated PBMCs. The inventors will then determine if DC-SIGN1 isoforms will present HIV Ba-L in trans to PHA and IL-2 activated PBMCs reflecting the natural process of DC capture and infection of resident CD4+ T cells. As a control, direct infection of K562 will be assessed in the absence of 293T transfected target cells. To determine if infection requires DC-SIGN capture of HIV-1, monoclonal antibodies to DC-SIGN1 will be incubated pre and post HIV-1 treatment. Blocking of HIV-1 infection in co-cultures would indicate a direct role for each of these DC-SIGN isoforms in participating in DC trans-infection. the DC-SIGN mAbs will be titraed (10-fold dilutions) to determine the minimal inhibitory concentration to competitively block bp120 binding to DC-SIGN.

### W. Example 23

### Isoforms that Lack a Transmembrane Domain Act as Soluble Inhibitors of DC-SIGN HIV-1 Binding

Soluble secreted sDC-SIGN will be produced using a baculovirus expression system (Invitrogen). DC-SIGN will be purified with anti-DC SIGN affinity columns using standard methodologies and its purity determined by SDS-PAGE. The ability of sDC-SIGN versions in blocking gp120 binding to DC-SIGN transfected 293T cells is then determined as described in the gp120 bead assay.

### X. Example 24

### Soluble DC-SIGN Isoforms Block the Transfer of Virus from DCs to T Cells

Metabolically labeled (35-S) HIV-1 will be solubilized, incubated with or without sDC-SIGN (10 μg/ml) followed by the addition sCD4 (10 μg/ml). The complexes are then immunoprecipitated with anti-CD4 non-blocking monoclonal antibodies which capture CD4-gp120 or with anti-DC-SIGN antibodies. The inventors have previously used several anti-CD4 antibodies which co-immunoprecipitate these complexes (Allan et al., 1990). Interference by sDC-SIGN presumably would result in a loss of gp120 immunoprecipitation. Based on these studies, the ability of sDC-SIGN to directly inhibit HIV-1 infection of immature DCs, T cell lines (Mot4, Hut78) and activated PBMCs will then be determined. Soluble DC-SIGN will be titrated to determine its potency in blocking HIV-1 infection by luciferase reporter HIV-1 pseudotypes and with infectious virus (R5 and X4 tropic viruses). The inventors will also perform the reverse precipitation in which sCD4 will first be incubated with gp120 followed by sDC-SIGN and the complexes immunoprecipitated with DC-SIGN antibody. Loss in co-immunoprecipitated gp120 would signify that sCD4 competes with DC-SIGN1 for binding gp120.

### Y. Example 25

### DC-SIGN Haplotypes are Associated with Altered Rates of HIV-1 Transmission and Disease Progression

Individuals vary in their susceptibility to infection with HIV-1. Ocasionally, hosts resist HIV-1 infection and, after infection has occurred, there is substantial variation in the rate of progression to AIDS (Dean et al., 1996; Dragic et al., 1996; Fowke et al., 1996; Kiu et al., 1996; Operskalski et al., 1997; Sherer and Clerici, 1996; Zagury et al., 1998; Zimmerman et al., 1997). A growing body of evidence suggests that genes that influence entry of HIV-1 into a cell or the host immune response to infection may play an important role in determining susceptibility to HIV-1 infection Berger et al., 1999; Gonzalez et al., 1999; Mummidi et al., 1998; Dean et al., 1996; Liu et al., 1996; Zagury et al., 1998; Zimmerman et al., 1997; Carrington et al., 1999; Martin et al., 1998; Huang et al., 1996; John et al., 2000; Easterbrook et al., 1999, Kaslow et al., 1996; Kostrikis et al., 1998, Kostrikis et al., 1999, McDermott et al., 199, Rizzardi et al., 1998; Tang et al., 1999). A powerfil approach to understanding the relationship between expression of a given gene and HIV-1 pathogenesis in vivo is to define the association between polymorphisms that may affect the expression of that gene and risk of transmission and/or clinical progression rate.

### Z. Example 26

### Assays for Single-Stranded Conformation Polymorphisms (SSCP) Across the Gene

An initial goal is to scan approximately 10 kb of the gene (coding+noncoding+promoter regions). Study of the pattern of the SSCP variations will allow us to determine a “bar code” distinguishing the extent of genetic versions of DC-SIGN1 in the study population. The inventors anticipate that by genetically profiling approximately 300 individuals from unrelated, ethnically-mixed (European-, African- and Hispanic American) normal donors, they will identify ˜60 individuals withthe broadest spectrum of variations in hte DC-SIGN1. The relevant polymorphic regions in the DC-SIGN1 gene from these 60 individuals will be sequenced to confirm these mutations. The inventors anticipate that SSCP analysis of 300 normal individuals should be of sufficient power to detect a broad range of genetic variants of the DC-SIGN1. It should be noted that a goal is to identify the most common genetic variants and not rare alleles. thus, this approach will not identify those variants whose allele frequencies are less than ∫0.4%. It should also be noted that via human genome project the complete DNA sequence surrounding the DC-SIGN1 locus is known, and this will greatly facilitate our the genotyping/sequencing work.

### AA. Example 27

### DC-SIGN1 Determinants of HIV Transmission

To test the hypothesis that specific DC-SIGN1 haplotypes determine, in part, the risk of HIV infection, the inventors will determine if specific haplotypes in the HIV cohort are under-represented (decreased transmission), equally-represented (non-protective) or over-represented (increased transmission). They anticipate that a detailed analysis of the haplotypes in the WHM and non-HIV cohort might reveal specific haplotypes that may play a role in transmission. This would not be an unanticipated finding considering the significant role that DC-SIGN1 is thought to play in HIV pathogenesis. It should be noted that the mechanisms of resistance to infection in the vast majority of highly exposed uninfected individuals remains unknown. For example, homozygosity for the inacting 32-bp deletion in DC-SIGN1 accounts for only 3% of all highly exposed but uninfected individuals. Genotyping will be conducted using PCR-RFLP and molecular beacon assays as described previously (Gonzalez et al., 1999).

### BB. Example 28

### DC-SIGN1 Determinants of HIV Progression

The statistical approaches to determine the association between disease progression (AIDS 1987 criteria and death) and specific haplotypes has been previously described (Gonzalez et al., 1999; Mummidi et al., 1998). The additive effects of and/or interaction between different haplotypes will be determined. Prognostic modeling that takes into consideration genotypic, immunologic (e.g. CD4), and viral (e.g. viral load) will also be performed.

The overall association between possession of a DC-SIGN1 haplotype/haplotype pair and risk of transmission may be evaluated using Chi-square or Fisher's exact test (SAS, version 8.0). When an overall difference was observed, the inventors adopted the strategy proposed by Fisher (1942) to determine which haplotype/haplotype was contributing to the overall effect. This approach corrects for multiple testing. To maintain power the classes of haplotype pairs that have less than the nine individuals are pooled together and included in the model. Time curves for progression to AIDS and death are prepared by the Kaplan-Meier (KM) method using SAS. Relative hazards are calculated using Cox proportional hazard models as described previously (Gonzalez et al., 1999; Mummidi et al., 1998).

Statistical analysis to determine if DC-SIGN1 genotypes are linked to differences in expression levels will include unpaired test (analysis of disease-modifying genotype versus those lacking this genotype). If the inventors find that they have to compare the levels of expression of several different genotypes, an ANOVA will be used to determine the overall difference followed by Scheffe's post hoc test (corrects for multiple testing). The correlation between DC-SIGN expression and HIV-trans-infection activity will be determined by Pearsons correlation coefficient (data is continuous variable). The inventors will also conduct sample size and power computations (Solo Power Analysis, BMDP Software) to determine the number of subjects that need to be studied to detect meaningful differences. These power calculations will depend significantly on the number of DC-SIGN1 genotypes that the inventors identify.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the squence of steps of the method described herein without departing from the concept, spirit and scope of the invention. More specifically, it will be apparent that certain agents which are both chemically and physiologically related may be s substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,340,535
U.S. Pat. No. 4,366,241
U.S. Pat. No. 4,472,509
U.S. Pat. No. 4,682,195
U.S. Pat. No. 4,683,202
U.S. Pat. No. 4,816,567
U.S. Pat. No. 4,867,973
U.S. Pat. No. 5,021,236
U.S. Pat. No. 5,466,468
U.S. Pat. No. 5,565,332
U.S. Pat. No. 5,645,897
U.S. Pat. No. 5,693,762
U.S. Pat. No. 5,705,629
U.S. Pat. No. 5,846,225
U.S. Pat. No. 5,846,233
U.S. Pat. No. 5,928,906
Abbondanzo, Ann Diagn Pathol, 3(5):318-327, 1990.
Abraham et al., Sience, 233:545-548, 1986
Adelman et al., Neuron, 9:209-216, 1992
Ahuja et al., J. Biol. Chem., 271:225-232, 1996.
Ahuja et al., J. Immunol., 161:868-876, 1998
Ahuja et al., J. Immunol., 163:3890-3897, 1999.
Ahuja, Methods Mol. Biol., 156:79-87; 67-77, 2001.
Alkhatib et al., J. Biol. Chem., 272:19771-19776, 1997.
Allan et al., AIDS Res. Hum. Retroviruses, 8:2011-2020, 1992.
Allan et al., Science, 247:1084-1088, 1990.
Allred et al., Arch Surg, 125(1):107-13, 1990.
Almendro et al., J. Immunol, 157(12):5411-5421, 1996.
Angel et al., Mol. Cell. Bioll, 7:2256, 1987.
Angel et al., Mol. Cell. Biol., 7:2256, 1987a.
Angel et al., Cell, 49:729, 1987b.
Anton van der Merwe et al., Semin. Immunol., 12:5-21, 2000.
Arinobu et al., Cell Immunol., 191:161-167, 1999.
Atchison and Perry, Cell, 46:253, 1986.
Atchison and Perry, Cell, 48:121, 1987.
Ausubel et al., In: Current Protocols in Molecular Biology, John Wiley & Sons, Inc, NY, 1994.
Ayehunie et al., AIDS Res. Hum. Retroviruses, 11:877-884, 1995.
Banchereau and Steinman, Nature, 392:245-252, 1998.
Banerji et al., Cell, 27(2 Pt 1):299-308, 1981.
Banerji et a;., Cell, 33(3):729-740, 1983.
Berger et al., Annu. Rev. Immunol., 17:657-700, 1999.
Berkhout et al., Cell, 59:273-282, 1989.
Black, Cell, 103:367-370, 2000.
Blanar et al., EMBO J., 8:1139, 1989.
Blaschke et al., J. Immunol. Methods, 246:79-90, 2000.
Blauvelt et al., J. Clin. Invest., 100:2043-2053, 1997.
Bleul et al., Proc. Natl. Acad. Sci. USA, 94:1925-1930, 1997.
Bloom and Beavo, Proc. Natl. Acad. Sci. USA, 93:14188-14192, 1996.
Bocek et al., J. Immunol., 158:3235-3243, 1997.
Bodine and Ley, EMBO J., 6:2997, 1987.
Boise et al., Cell, 74:597-68, 1993.
Boshart et al., Cell, 41:521, 1985.
Bosze et al., EMBO J., 5(7):1615-1623, 1986.
Braddock et al., Cell, 58:269, 1989.
Brodeur, et al., In: Monoclonal Antibody Production Techniques and Applications, 51-63, Marcel Dekker, Inc. NY, 1987
Brown et al., Immunol Ser, 53:69-82, 1990.
Bulla and Siddiqui, J. Virol., 62:1437, 1986.
Cameron et al., AIDS Res. Hum. Retroviruses, 10:61-71, 1994.
Cameron et al., J. Leukoc. Biol., 59:158-171, 1996.
Cameron et al., Science, 257:383-387, 1992.
Campbell and Villarreal, Mol. Cell. Biol., 8:1993, 1988.
Campere and Tilghman, Genes and Dev., 3:537, 1989.
Campo et al., Nature, 303:77, 1983.
Canque et al., Blood, 93:3866-3875, 1999.
Carbonelli et al., FEMS Microbiol Lett, 177(1):75-82, 1999.
Celander and Haseline, J. Virology, 61:269, 1987.
Celander et al., J. Virology, 62:1314, 1988.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Chatterjee et al., Proc. Natl. Acad Sci. U.S.A., 86:9114, 1989.
Chess et al., Cell, 78:823-834, 1994.
Choi et al., Cell, 53:519, 1988.
Cimarelli et al., J. Acquir. Immune Defic. Synot., 7:230-235, 1994.
Clinton et al., Biochem. Biophys. Res. Commun., 158:855-862, 1989.
Cocea, Biotechniques, 23(5):814-816, 1997.
Cohen et al., J. Cell. Physiol., 5:75, 1987.
Cook and Tomlinson, Immunol Today, 16:237-242, 1995.
Cooper and Barondes, J. Cell Biol., 110:1681-1691, 1990.
Corradi et al., J. Neurosci, 17:1406-1415, 1997.
Costa et al., Mol. Cell. Biol., 8:81, 1988.
Cote et al., J Biol. Chem., 2000.
Cripe et al., EMBO J., 6:3745, 1987.
Culotta and Harner, Mol. Cell. Biol., 9:1376, 1989.
Curtis et al., Proc. Natl. Acad. Sci. USA, 89:8356-8380, 1992.
Dandolo et al., J. Virology, 47:55-64, 1983.
Davis and Bjorkman, Nature, 334:395-402, 1988.
de Melker and Sonnenberg, Bioessays, 21:49-509, 1999.
De Villers et al., Nature, 312(5991):242-246, 1984.
Dean et al., Science, 273:1856-1862, 1996.
Delespesse et al., Adv. Immunol., 49,149-191, 1991.
Deschamps et al., Science, 230:1174-1177, 1985.
Dietz et al., Biochem. Biophys. Res. Commun., 275:731-738, 2000.
Dragic et al., Nature, 381:667-673, 1996.
Drickamer and Taylor, Annu. Rev. Cell Biol., 9:237-264, 1993.
Drickamer, Curr. Opin. Struct. Biol., 5:612-416, 1995.
Drickamer, Curr. Opin. Struct. Boil., 9:585-590, 1999.
Easterbrook et al., J. Infect. Dis., 80:1096-1105, 1999.
Edbrooke et al., Mol. Cell. Biol., 9:1908, 1989.
Edlund et al., Science, 230:912-916, 1985.
Eissa et al., J. Biol. Chem., 271:27184-27187, 1996.
EP Appl. 266,032
Fehg and Holland, Nature, 334:6178, 1988.
Fichter et al., Oncogene, 14:2817-2824, 1997.
Firak and Subramanian, Mol. Cell. Biol., 6:3667, 1986.
Fisher, In: The Design of Experiments, 3d Edition, Edinburgh, Oliver and Boyd, 1942.
Foecking and Hofstetter, Gene, 45(1):101-105, 1986.
Fowke et all., Lancet, 348:1347-1351, 1996.
Frank et al., J. Virol., 73:3449-3454, 1999.
Frankel et al., Sience, 272:115-117, 1996.
Fujita et al., Cell, 49:357, 1987.
Garcia-Sanz and Lenig, J. Exp. Med., 184:159-164, 1996.
Garcia-Sanz et al., FASEB J., 12:299-306, 1998.
Geijtenbeek et al., Cell, 100:575-585, 2000b
Geijtenbeek et al., Cell, 100:587-597, 2000a
Geijtenbeek et al., Blood, 94:754, 1999.
Gilles et al., Cell, 33:717, 1983.
Gloss et al., EMBO J., 6:3735, 1987.
Godbout et al. Mol. Cell. Biol., 8:1169, 1988.
Gonzalez et al., Proc. Natl. Acad. Sci. USA, 96:12004-12009, 1999.
Goodall et al., Proc. Natl. Acad. Sci. USA, 83:8928, 1986.
Goodbourn and Maniatis, Proc. Natl. Acad. Sci. USA, 85:1447, 1988.
Goodbourn et al., Cell, 45:601, 1986.
Gordon, Immunol. Today, 15:411-417, 1994.
Grabowski, Cell, 92:709-712, 1998.
Granelli-Piperno et al., Curr. Biol., 9:21-29, 1999.
Granelli-Piperno et al., J. Exp. Med., 184:2433-2438, 1996.
Granelli-Piperno et al., Proc. Natl. Acad. Sci. USA, 92:10944-10948, 1995.
Greene et al., Immunology Today, 10:272, 1989
Grosschedi and Baltimore, Cell, 41:485, 1985.
Harlow and Lane, Antbodies: A Laboratory Manual, Cold Spring Harbor Laboratory, 1988.
Hart, Blood, 90:3245-3287, 1997.
Haslinger and Karin, Proc. Natl. Acad. Sci. USA, 82:8572, 1985.
Hauber and Cullen, J. Virology, 62:673, 1988.
Hebert, Biosci. Rep., 20:213-237, 2000.
Hen et al., Nature, 321:249, 1986.
Hensel et al., Lymphokine Res., 8:347, 1989.
Herr and Clarke, Cell, 45:461, 1986.
Hill et al., J. Biol. Chem., 268:726-731, 1993.
Hirochika et al., J. Virol., 61:2599, 1987.
Hirsch et al., Mol. Cell. Biol., 10:1959, 1990.
Hadik et al., J. Immunol., 163:2306-2313, 1999.
Holbrook et al., Virology, 157-211, 1987.
Horlick and Benfield, Mol. Cell. Biol., 9:2396, 1989.
Hu et al., J. Virol., 74:6087-6095, 2000.
Huang et al., Cell, 27:245, 1981.
Huang et al., Nat. Med., 2:1240-1243, 1996.
Hug et al., Mol. Cell. Biol., 8:3065, 1988.
Hwang et al., Mol. Cell. Biol., 10:585, 1990.
Imagawa et al., Cell, 51:251, 1987.
Imataka et al., J. Biol. Chem., 269:20668-20673, 1994.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaye et al., Science, 233:541-545, 1986.
Jaynes et al., Mol. Cell. Biol., 8:62, 1988.
Jiang and Wu, Proc. Soc. Exp. Biol. Med., 220:6472, 1999.
John et al., J. Virol., 74:5736-5739, 2000.
Johnson et al., Anal. Biochem., 278:175-184, 2000.
Johnson et al., Mol. Cell. Biol., 9:3393, 1989.
Jonuleit et al., Eur. J. Immunol., 27:3135-3142, 1997.
Kacani et al., J. Viron., 72:6671-6677, 1998.
Kadesch and Berg, Mol. Cell. Biol., 6:2593, 1986.
Kafert et al., Anal. Biochem., 269:210-213, 1999.
Karin et al., Mol. Cell. Biol., 7:606, 1987.
Karin et al., Mol. Cell. Biol., 7:606, 1987.
Kaslow et al., Nat. Med., 2:405-411, 1996.
Katinka et al., Cell, 20:393, 1980.
Kawamoto et al., Mol. Cell. Biol., 8:267, 1988.
Kikutani et al., Ciba Found. Symp., 147:23-31, 1989.
Kiledijan et al., Mol. Cell. Biol., 8:145, 1988.
Klaassen, In: The Pharmacological Basis of Therapeutics, Goodman and Gilman, Eds., Pergamon Press, 8Ed., 49-61, 1990.
Klamut et al., Mol. Cell. Biol., 10:193, 1990.
Knight and Patterson, Annu. Rev. Immunol., 15:593-615, 1997.
Koch et al., Mol. Cell. Biol., 9:303, 1989.
Kohmura et al., Neuron, 20:1137-1151, 1998.
Kostrikis et al., J. Virol., 73:10264-10271, 1999.
Kostrikis et al., Nat. Med., 4:350-353, 1998.
Kozbor, J. Immunol., 133, 3001, 1984.
Kraus et al., FEBS Lett., 428(3):165-170, 1998.
Kriegler and Botchan, In: Eukaryotic Viral Vectors, Y. Gluzman, ed., Cold Spring Harbor: Cold Spring Harbor Laboratory, N.Y., 1982.
Kriegler and Botchan, Mol. Cell. Biol., 3:325, 1983.
Kriegler et al., Cell, 38:483, 1984a.
Kriegler et al., Cell, 53:45, 1988.
Kuhl et al., Cell, 50:1057, 1987.
Kunz et al., Nucl. Acids Res., 17:1121, 1989.
Lareyre et al., J. Biol. Chem., 274(12):8282-8290, 1999.
Larsen et al., Proc. Natl. Acad. Sci. USA, 83:8283, 1986.
Laspia et al., Cell, 59:283, 1989.
Latimer et al., Mol. Cell. Biol., 10:760, 1990.
Lee et al., DNA Cell Biol, 16(11):1267-75, 1997.
Lee et al., Nature, 294:228, 1981.
Lee et al., Nucleic Acids Res., 12:4191-206, 1984.
Lee et al., Proc. Natl. Acad. Sci. USA, 96:5215-5220, 1999.
Levenson et al., Hum Gene Ther, 9(8):1233-6, 1998.
Levinson et al., Nature, 295:79, 1982.
Lien et al., Mol. Cell Biol., 17(4):2279-2290, 1997.
Lin et al., Mol. Cell. Biol., 10:850, 1990.
Liu et al., Cell, 86:367-77, 1996.
Lopez, Annu. Rev. Genet., 32:279-305, 1998.
Luo and Skalnik, J. Biol. Chem., 271:18203-18210, 1996a.
Luo and Skalnik, J. Biol. Chem., 271:23445-23451, 1996b.
Luo et al., J. Biol. Chem., 273:28486-28495, 1998.
Luria et al., EMBO J., 6:3307, 1987.
Lusky and Botchan, Proc. Natl. Acad. Sci. USA, 83:3609, 1986.
Lusky et al., Mol. Cell. Biol. 3:1108, 1983.
Macejak and Sarnow, Nature, 353:9094, 1991.
Mackay et al., J. Cell Biol., 124:71-82, 1994.
Majors and Vamus, Proc. Nat'l Acad. Sci. USA, 80:5866, 1983.
Mangano et al., J. Infect., 183:1574-1585, 2001.
Maniatis, et al., In: Molecular Cloning, A Laboratory Manual, Cold Spring Harbor Press, Cold Spring Harbor, N.Y., 1990.
March et al., Nature, 315:641-647, 1985.
Marrack et al., Curr. Opin. Immunol., 12:206-209, 2000.
Martin et al., Science, 282:1907-1911, 1998.
Masuier et al., J. Virol., 72:7822-789, 1998.
May et al., Natue, 406(6791):82-86, 2000.
McDermott et al., Lancet, 352:866-870, 1998.
McNeall et al., Gene, 76:81, 1989.
Merck Index, 11^{th} Edition, Susan Budavari (Ed.,), Merck & Col, Inc., NJ, 1989.
Metheny and Skuse, Exp. Cell Res., 228:44-49, 1996.
Miksicek et al., Cell, 46:203, 1986.
Mikufits et al., FASEB J., 14:1641-1652, 2000.
Miller et al., Lab. Invest., 68:129-145, 1993.
Missler and Sudhof, Trends Genet., 14:20-26, 1998.
Mordacq and Linzer, Genes and Dev., 3:760, 1989.
Moreau et al., Nucl. Acids Res., 9:6047, 1981.
Muesing et al., Cell, 48:691, 1987.
Mullin et al., J. Biol. Chem., 272:5668-5681, 1997.
Mummidi et al., Nat Med., 4:786-793, 1998.
Nakabeppu and Nathans, Cell, 64:751-759, 1991.
Nakamura et al., In: Handbook of Experimental Immunology (4^{th}Ed.), Weir et al., (eds). 1:27, Blackwell Scientific Publ., Oxford, 1987.
Neefjes and Ploegh, Immunol. Today, 13:179-184, 1992.
Newell, AIDS, 12:831-837, 1998.
Ng et al., Nuc. Acids Res., 17:601, 1989.
Nomoto et al., Gene, 236(2):259-71, 1999.
Ondek et al., EMBO J., 6:1017, 1987.
Operskalski et al., J. Acquir. Immune Defic. Syndr. Hum. Retrovirol., 15:145-150, 1997.
Omitz et al., Mol. Cell. Biol., 7:3466, 1987.
Oz et al., J. Vasc. Surg., 11(5):718-725, 1990.
Palmiter et al., Nature, 300:611, 1982.
Patterson and Knight, J. Gen. Virol., 68:1177-1181, 1987.
PCT Appl. WO 84/03564
Pech et al., Mol. Cell. Biol., 9:396, 1989.
Pelletier and Sonenberg, Nature, 334:320-325, 1988.
Perez-Stable and Constantini, Mol. Cell. Biol., 10:1116, 1990.
Physicians Desk Reference
icard and Schaffner, Nature, 307:83, 1984.
Pinchuk et al., Immunity, 1:317-325, 1994.
Pinkert et al., Genes and Dev., 1:268, 1987.
Pohlmam et al., Proc. Natl. Acad. Sci. USA, 98:2670-2675, 2001.
Ponta et al., Proc. Nat'l Acad. Sci. USA, 82:1020, 1985.
Pope et al., Cell, 78:389-398, 1994.
Pope et al., J. Exp. Med., 182:2045-2056, 1995.
Porton et al., Mol. Cell. Biol., 10:1076, 1990.
Queen and Baltimore, Cell, 35:741, 1983.
Quinn et al., Mol. Cell. Biol., 9:4713, 1989.
Quinones et al., J. Exp. Med., 192:507-516,2000.
Rao et al., J. Biol. Chem., 268:22164-22169, 1993.
Redondo et al., Sience, 247:1225, 1990.
Reece et al., J. Exp. Med, 187:1623-1631, 1998.
Reisman and Rotter, Mol. Cell., Biol., 9:3571, 1989.
Remington's Pharmaceutical Sciences, 15^{th} Edition, Chapter 61, pages 1035-1038 and 1570-1580.
Resendez Jr. et al., Mol. Cell. Biol., 8:4579, 1988.
Reynes et al., J. Infect. Dis., 181:927-932, 2000.
Ripe et al., Mol. Cell. Biol., 9:2224, 1989.
Rittling et al., Nuc. Acids Res., 17:1619, 1989.
Rizzardi et al., Nat. Med., 4:252-253, 1998.
Roca et al., Am. J. Pathol, 153:183-190, 1998.
Rosen et al., Cell, 41:813, 1988.
Ruan et al., J. Biol. Chem., 269:17905-17910, 1994.
Sakai et al., Genes and Dev., 2:1144, 1988.
Sambrook et al., In: Molecular cloning, Cold Spring Harbor Laboratory Press, Cold Spring Harbor, N.Y., 2001.
Sambrook et al., Molecular Cloning: A Laboratory Manual, 3^{red} Ed., Cold Spring Harbor Press, Cold Spring Harbor, N.Y., 2001.
Sancho et al., J. Immunol., 165:3868-3875, 2000.
Satake et al., J. Virology, 62:970, 1988.
Sato et al., J. Exp. Med., 192:205-218, 2000.
Schafner et al., J. Mol. Biol., 201:81, 1988.
Schmucker et al., Cell, 101:671-684, 2000.
Searle et al., Mol. Cell. Biol., 5:1480, 1985.
Shaham and Horvitz, Cell 86:201-208, 1996.
Shapiro and Colman, Neuron., 23:427-430, 1999.
Sharp and Marciniak, Cell, 59:229, 1989.
Sharp, Cell, 77:805-815, 1994.
Shaul and Ben-Levy, EMBO J., 6:1913, 1987.
Shearer and Clerici, Immunol. Today, 17:21-24, 1996.
Sherman et al., Mol. Cell. Biol., 9:50, 1989.
Sleigh and Lockett, J. EMBO, 4:3831, 1985.
Smith and Valcarcel, Trends Biochem. Sci., 25:381-388, 2000.
Soilleux et al., J. Immunol., 165:2937-2942, 2000.
Soto-Ramirez et al., Science, 271:1291-1293, 1996.
Spalholz et al., Cell, 42:183, 1985.
Spandau and Lee, J. Virology, 62:427, 1988.
Spandidos and Wilkie, EMBO J., 2:1193, 1983.
Spira et al., J. Exp. Med., 183:215-125, 1996.
Steinman, Cell, 100:491-494, 2000.
Stephens and Hentschel, Biochem. J., 248:1, 1987.
Stuart et al., Nature 317:828, 1985.
Sullivan and Peterlin, Mol. Cell. Biol., 7:3315, 1987.
Suresh et al., Methods in Enzymology, 121:210, 1986.
Suter et al., nucleic Acids Res., 15:7295-7308, 1987.
Swartzendruber and Lehman, J. Cell. Physiology, 85:179, 1975.
Takebe et al., Mol. Cell. Biol., 8:466, 1988.
Tang et al., Genes and Immunity, 1:20-27, 1999.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Thomas and Lipsky, J. Immunol., 153:4016-4028, 1994.
Tone et al., Proc. Natl. Acad. Sci. USA, 98:1751-1756, 2001.
Treisman, Cell, 42:889, 1985.
Tronche et al., Mol. Biol. Med., 7:173, 1990.
Trudel and Constantini, Genes and Dev. 6:954, 1987.
Tsumaki et al., J. Biol. Chem., 273(36281-22864, 1998.
Tsunetsugu-Yokota et al., J. Virol., 69:4544-4547, 1995.
Tsunetsugu-Yokota et al., Virology, 239:259-268, 1997.
Tsytsikov et al., J. Biol. Chem., 271:23055-2360, 1996.
Tyndell et al., Nuc. Acids. Res., 9:6231, 1981.
Uemura, Cell, 93:1095-1098, 1998.
Ullrich et al., Neuron., 14:497-507, 1995.
Vannice and Levinson, J. Virology, 62:1305, 1988.
Vasseur et al., Proc Natl. Acad. Sci. U.S.A., 77:1068, 1980.
Walker and Rigley, J. Immunol. Methods, 239:167-179, 2000.
Wang and Calame, Cell, 47:241, 1986.
Wang et al., Cell, 78:739-750, 1994.
Wang et al., Cell, 93:47-60, 1998.
Weber et al., Cell, 36:983, 1984.
Weinberger et al., Mol. Cell. Biol., 8:988, 1984.
Weis and Drickamer, Annu. Rev. Biochem., 65:441-473, 1996.
Weis et al., Immunol. Rev., 163:19-34, 1998.
Weissman and Fauci, Clin. Microbiol Rev., 10:358-367, 1997.
Weissmn et al., J. Immunol., 155:4111-4117, 1995.
Weissman et al., Proc. Natl. Acad. Sci. USA, 92:826-830, 1995.
Williams et al., J. Mol. Biol., 264:220-232, 1196.
Winoto and Baltimore, Cell 59:649, 1989.
Wu and Maniatis, Cell 97:779-790, 1999.
Wu et al., Nature, 384:179-183, 1996.
Wu et al., Biochem. Biophys. Res. Commun. 233(1):221-226, 1997.
Ying et al., J. Immunol., 155:1637, 1995.
Yokota et al., Cell, 55:611-618, 1988.
Yokoyama-Kobayashi et al., Gene, 228:161-167, 1999.
Yoshikawa et al., Mol. Immunol., 36:1223-1233, 1999.
Yutzey et al., Mol. Cell. Biol, 9:1397, 1989.
Zagury et al., Proc. Natl. Acad. Sci. USA, 95:3857-3861, 1998.
Zaitseva et al., Nat. Med., 3:1369-1375, 1997.
Zhao-Emonet et al., Biochim Biophys Acta, 1442(2-3):109-119, 1998.
Zimmerman et al., Mol. Med., 3:23-36, 1997.

1-127. (canceled)

128. An isolated and purified nucleic acid encoding an isoform of DC-SIGN1, DC-SIGN2 or DC-SIGN 3.

129. The nucleic acid of claim 128, wherein the nucleic acid encodes an isoform shorter than the fill length DC-SIGN1, DC-SIGN2 or DC-SIGN3 isoforms.

130. The nucleic acid of claim 128, wherein the nucleic acid encodes an isoform comprising a carbohydrate recognition domain (CRD).

131. The nucleic acid of claim 130, wherein the isoform further comprises a lectin binding domain.

132. The nucleic acid of claim 131, wherein the isoform further comprises a neck repeat region comprising from 1 to 8 repeats.

133. The nucleic acid of claim 132, wherein the isoform further comprises a transmembrane domain.

134. The nucleic acid of claim 133, wherein the isoform further comprises a cytoplasmic (CYT) domain.

135. The nucleic acid of claim 132, wherein the isoform does not comprise a transmembrane domain.

136. The nucleic acid of claim 135, wherein the isoform further comprises a CYT domain.

137. The nucleic acid of claim 128, wherein the nucleic acid encodes a membrane bound isoform of DC-SIGN1, DC-SIGN2 or DC-SIGN3.

138. The nucleic acid of claim 128, wherein the nucleic acid encodes a soluble isoform of DC-SIGN1, DC-SIGN2 or DC-SIGN3.

139 The nucleic acid of claim 128, wherein the nucleic acid encodes an isoform of DC-SIGN1.

140. The nucleic acid of claim 128, wherein the nucleic acid encodes an isoform of DC-SIGN2.

141. The nucleic acid of claim 128, wherein the nucleic acid encodes an isoform of DC-SIGN3.

142. The nucleic acid of claim 128, further defined as comprised in a cell transformed with the nucleic acid.

143. An isolated and purified isoform of DC-SIGN1, DC-SIGN2 or DC-SIGN 3.

144. The isoform of claim 143, wherein the isoform is shorter than the full length DC-SIGN1, DC-SIGN2 or DC-SIGN3 isoforms.

145. The isoform of claim 143, wherein the isoform comprises a carbohydrate recognition domain (CRD).

146. The isoform of claim 145, wherein the isoform further comprises a lectin binding domain.

147. The isoform of claim 146, wherein the isoform further comprises from 1 to 8 neck repeats.

148. The isoform of claim 147, wherein the isoform further comprises a transmembrane domain.

149. The isoform of claim 148, wherein the isoform further comprises a CYT domain.

150. The isoform of claim 147, wherein the isoforin does not comprise a transmembrane domain.

151. The isoform of claim 150, wherein the isoform further comprises a CYT domain.

152. The isoform of claim 143, wherein the isoform is a membrane bound isoform of DC-SIGN1, DC-SIGN2 or DC-SIGN3.

153. The isoform of claim 143, wherein the isoform is a soluble isoform of DC-SIGN1, DC-SIGN2 or DC-SIGN3.

154. The isoform of claim 143, wherein the isoform is an isoform of DC-SIGN1.

155. The isoform of claim 143, wherein the isoform is an isoform of DC-SIGN2.

156. The isoform of claim 143, wherein the isoform is an isoform of DC-SIGN3.

157. A method for treating disease comprising administering a therapeutically effective amount of an isoform of DC-SIGN1, DC-SIGN2 or DC-SIGN-3 to a subject in need of such treatment.

158. The method of claim 157, wherein the disease is selected from cancer, viral infection, or non-HIV induced immunosuppression.

159. The method of claim 157, wherein the isoform is a DC-SIGN1 isoform.

160. The method of claim 157, wherein the isoform is a DC-SIGN2 isoform.

161. The method of claim 157, wherein the isoformn is a DC-SIGN3 isoform.

162. The method of claim 157, wherein the isoform is a soluble isoform.

163. A method of modulating an immune response comprising providing an amount of a DC-SIGN isoform sufficient to enhance or inhibit the immune response.

164. The method of claim 163, wherein the amount is sufficient to enhance the immune response.

165. The method of claim 163, wherein the amount is sufficient to inhibit the immune response.

166. The method of claim 163, wherein the immune response is a T-cell mediated immune response.

167. A polypeptide comprising a fusion of extracellular, CDR, neck repeat, transmembrane, intracellular domain, and ICAM binding domains of a DC-SIGN isoform in which at least one domain is taken from a DC-SIGN isoform other than the DC-SIGN isoforms from which the remaining domains are taken.

168. A nucleic acid encoding a polypeptide comprising a fusion of extracellular, CDR, neck repeat, transmembrane, intracellular domain, and ICAM binding domains of a DC-SIGN isoform in which at least one domain is taken from a DC-SIGN isoform other than the DC-SIGN isoforms from which the remaining domains are taken.

169. A method of modulating resistance to viral infection comprising identifying a subject at risk for a viral infection and administering to the subject a composition comprising a DC-SIGN isoform, a fusion protein containing a DC-SIGN domain, or an antibody to DC-SIGN in an amount sufficient to alter the resistance of the subject to the viral infection.

170. A method of augmenting transformation of ICAM expressing cells comprising:
a) obtaining a cell that expresses ICAM on its surface;
b) obtaining a viral vector; and
c) contacting the cell of step (b) with the vector of step (a) in the presence of a DC- SIGN isoform such that the vector is incorporated into the cell.

171. A method of assaying for susceptibility to disease comprising:
a) obtaining a sample from a subject to be assayed;
b) identifying the DC-SIGN type present in the sample; and
c) determining the susceptibility of the subject to disease based upon a correlation of DC-SIGN type and susceptibility.

172. A method of treating disease comprising:
a) identifying a subject in need of treatment;
b) obtaining a cell;
c) transforming the cell with a nucleic acid encoding a DC-SIGN isoform; and
d) administering the cell to the subject.
